(19) United States
(12) Patent Application Publication
(10) Pub. No.: US 2008/0004268 A1
(43) Pub. Date: Jan. 3, 2008

(54) INHIBITORS OF SOLUBLE ADENYLATE CYCLASE

(76) Inventors: Duy NGUYEN, Berlin (DE); Anne MENGEL, Berlin (DE); Martin FRITSCH, Berlin (DE); Gernot LANGER, Berlin (DE); Ulf BOEMER, Glienicke/Nordbahn (DE); Knut EIS, Berlin (DE); Bernd MENZENBACH, Jena (DE); Bernd BUCHMANN, Hohen Neuendorf (DE); Seock-Kyu KHIM, Orinda, CA (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/835,164

(22) Filed: Aug. 7, 2007

Related U.S. Application Data
(62) Division of application No. 11/233,533, filed on Sep. 23, 2005.

(30) Foreign Application Priority Data
Sep. 24, 2004 (DE) 102004047272.6

(51) Int. Cl. A61K 31/5377; A61K 31/454; A61K 31/405; C07D 413/02; C07D 405/02; C07D 403/02

(52) U.S. Cl. 514/232.5; 514/323; 514/414; 514/337; 544/141; 546/201; 548/465

(57) ABSTRACT

The invention relates to compounds of general formula I as well as their production and use as a medication.

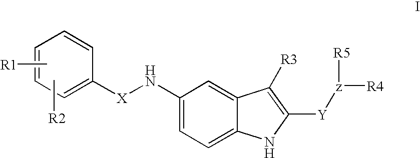

This application claims the benefit of the filing date of U.S. Provisional Application Ser. No. 60/614,527 filed Oct. 1, 2004 which is incorporated by reference herein.

This invention relates to inhibitors of soluble adenylate cyclase, its production as well as its use for the production of a pharmaceutical agent for contraception.

There are currently a number of modern contraceptive methods available for women; for male birth control, however, only very few methods are available (condom and sterilization). The development of new reliable agents for male birth control is absolutely necessary. In this connection, infertility produced by a “male pill” should be completely reversible and just as effective as the existing methods that are available to women. The infertility should set in relatively quickly and last as long as possible. Such contraceptive methods should not have any side effects; in addition to hormonal preparations, these can also be non-hormonal preparations in this connection. A possible starting point is the regulation of the activity of an enzyme, which plays an important role in the fertilization of an ovocyte, the soluble adenylate cyclase (sAC). This enzyme is expressed mainly in the testicular stem cells and is present in mature sperm.

In 1999, the authors Levin and Buck (Proc. Natl. Acad. Sci. USA 96 (1): 79-84) were able to purify and to clone an isoform of the sAC from the testes of rats.

The recombinant enzyme of rats can be stimulated by bicarbonate. By means of antibodies, it was possible to demonstrate that the catalytic domain of the enzyme is located in the testes, sperm, kidneys and the choroid plexus. These disclosures are the subject matter of application WO01/85753, which was granted in the U.S. (U.S. Pat. No. 6,544,768).

In WO01/21829 (Conti et al.), isolated polynucleotide sequences that code for the human isoform of sAC, isolated sAC polypeptides and test systems are claimed with whose help substances can be identified that inhibit the activity of sAC. The possibility of using these substances to reduce the number of motile sperm cells in a reversible manner as well as their use as agents for male birth control are disclosed.

The John Herr group showed the isolation and characterization of the human isoform of sAC from sperm. In WO 02/20745, in addition to nucleic acids, the test systems that also code for sAC are claimed, with whose aid substances can be identified that modulate the expression or the activity of the human sAC. Such compounds could selectively inhibit, for example, the activity of sAC; this had the result that the sperm cells lose the ability to fertilize an ovocyte. These inhibitors of sAC therefore could be used as pharmaceutical agents for non-hormonal contraception.

The already known inhibitors of sAC indicate specific problems, however: catechol estrogens (T. Braun, Proc Soc Exp Biol Med 1990, 194(1): 58ff) and gossypol (K. L. Olgiati Arch Biochem Biophys 1984, 231(2): 411 ff) are inherently toxic, while adenosine analogs inhibit with only very weak action (M. A. Brown and E. R. Casillas J Androl 1984, 5:361 ff). The inhibitors (IC₅₀≦10 μmol) of the recombinant human sAC, which are described by Zippin et al. (J. H. Zippin et al. J Cell Biol 2004, 164(4): 527 ff), are somewhat more potent.

To be able to make an agent for male birth control available, there is an increasing need for substances that are reversible, quick and successfully result in infertility.

This object is achieved by the provision of the compounds of general formula I

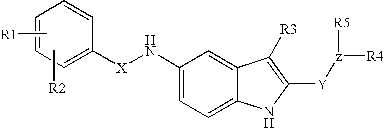

in which
R¹ stands for hydrogen, halogen, CF₃, C₃-C₆-cycloalkyl, which optionally is polysaturated and optionally is polysubstituted, or for the group C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl, C₁-C₆-aryl-C₁-C₆-alkyl or CF₃, in which C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl or C₁-C₆-aryl-C₁-C₆-alkyl optionally can be interrupted in one or more places, in the same way or differently, by oxygen, sulfur or nitrogen, or for the group sulfonyl-C₁-C₆-alkyl, sulfonamide, or cyano,
R² stands for halogen, CF₃, C₃-C₆-cycloalkyl, which optionally is polysaturated and optionally is polysubstituted, or for the group C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl, C₁-C₆-aryl-C₁-C₆-alkyl or CF₃, in which C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl or C₁-C₆-aryl-C₁-C₆-alkyl optionally can be interrupted in one or more places, in the same way or differently, by oxygen, sulfur or nitrogen, or for the group sulfonyl-C₁-C₆-alkyl, sulfonamide, or cyano,
R³ stands for, C₆-C₁₂-aryl, which optionally can be substituted in one or more places, in the same way or differently, with halogen, with C₁-C₆-alkyl or C₁-C₆-acyl, which optionally can be substituted in one or more places, or can be substituted with C₁-C₆-alkoxy, hydroxy, cyano, CO₂—(C₁-C₆-alkyl), N—(C₁-C₆-alkyl)₂, CO—NR⁴R⁵ or with CF₃, for C₅-C₁₂-heteroaryl, which optionally can be substituted in one or more places, in the same way or differently, with halogen, with C₁-C₆-alkyl, C₁-C₆-acyl, C₁-C₆-alkoxy, hydroxy, cyano, CO₂—(C₁-C₆-alkyl), N—(C₁-C₆-alkyl)₂, CO—NR⁴R⁵ or with CF₃, or for C₃-C₆-cycloalkyl, which optionally can be substituted in one or more places, in the same way or differently, with halogen, CF₃, hydroxy, cyano, CO₂—(C₁-C₆-alkyl), C₁-C₆-alkyl, C₁-C₆-acyl, N—(C₁-C₆-alkyl)₂, CO—NR⁴R⁵ or C₁-C₆-alkoxy,
R⁴ stands for hydrogen, C₃-C₆-cycloalkyl, which optionally is substituted in one or more places, in the same way or differently, with C₁-C₆-alkyl, C₁-C₆-acyl, C₁-C₆-alkoxy or CF₃, for C₆-C₁₂-aryl, which optionally is substituted in one or more places, in the same way or differently, with halogen, with C₁-C₆-alkyl, C₁-C₆-acyl, C₁-C₆-alkoxy, N—C₁-C₆-alkyl-C₁-C₆-alkyl, CF₃ or cyano, or for C₅-C₁₂-heteroaryl, which optionally is substituted in one or more places, in the same way or differently, with halogen, with C₁-C₆-alkyl, C₁-C₆-acyl, C₁-C₆-alkoxy, N—C₁-C₆-alkyl-C₁-C₆-alkyl, CF₃ or cyano, or for C₁-C₆-alkyl, which can be substituted in any way desired,
R⁵ stands for hydrogen, C₃-C₆-cycloalkyl, which optionally is substituted in one or more places, in the same way or differently, with C₁-C₆-alkyl, C₁-C₆-acyl, C₁-C₆-alkoxy or CF₃, for C₆-C₁₂-aryl, which optionally is substituted in one or more places, in the same way or differently, with halogen, with C₁-C₆-alkyl, C₁-C₆-acyl, C₁-C₆-alkoxy, N—C₁-C₆-alkyl-C₁-C₆-alkyl, CF₃ or cyano, or for C₅-C₁₂-heteroaryl, which optionally is substituted in one or more places, in the same way or differently, with halogen, with C₁-C₆-alkyl, C₁-C₆-acyl, C₁-C₆-alkoxy, N—C₁-C₆-alkyl-C₁-C₆-alkyl, CF₃ or cyano, or for C₁-C₆-alkyl, which can be substituted in any way desired,
R⁴ and R⁵ together form a 5- to 8-membered ring, which can contain additional heteroatoms, and
X stands for the groups sulfonyl, (CH₂)ₙ or carbonyl,
Y stands for carbonyl or (CH₂)ₙ,
Z stands for nitrogen,
n stands for 0-4,
   as well as their isomers, diastereomers, enantiomers and salts that overcome the known disadvantages and exhibit improved properties, i.e., good effectiveness, good solubility and stability.

The compounds according to the invention inhibit the soluble adenylate cyclase and thus prevent sperm capacitation and thus are used for male birth control.

Alkyl is defined in each case as a straight-chain or branched alkyl radical, such as, for example, methyl, ethyl, propyl, isopropyl, butyl, isobutyl, sec-butyl, tert-butyl, pentyl, isopentyl and hexyl.

Alkoxy is defined in each case as a straight-chain or branched alkoxy radical, such as, for example, methoxy, ethoxy, n-propoxy, iso-propoxy, n-butoxy, sec-butoxy, iso-butoxy, tert-butyloxy, pentoxy, iso-pentoxy and hexoxy.

Acyl is defined in each case as a straight-chain or branched radical, such as, for example, formyl, acetyl, propionyl, butyroyl, iso-butyroyl, valeroyl and benzoyl.

Cycloalkyls are defined as monocyclic alkyl rings such as cyclopropyl, cyclobutyl, cyclopentyl, and cyclohexyl.

Instead of the carbon atoms, the cycloalkyl radicals can contain one or more heteroatoms, such as oxygen, sulfur and/or nitrogen. Preferred are those heterocycloalkyls with 3 to 6 ring atoms. The ring systems, in which optionally one or more possible double bonds can be contained in the ring, are defined as, for example, cycloalkenyls, such as cyclopropenyl, cyclobutenyl, cyclopentenyl, cyclopentadienyl, cyclohexenyl, or cycloheptenyl, whereby the linkage both to the double bond and to the single bonds can be carried out.

Halogen is defined as fluorine, chlorine, bromine or iodine in each case.

In each case, the aryl radical comprises 6-12 carbon atoms and can be, for example, benzocondensed. For example, the following can be mentioned: phenyl, tropyl, cyclooctadienyl, indenyl, naphthyl, biphenyl, florenyl, anthracenyl, etc.

The heteroaryl radical comprises 5-16 ring atoms in each case and instead of the carbon can contain one or more, same or different, heteroatoms, such as oxygen, sulfur or nitrogen, in the ring, and can be monocyclic, bicyclic or tricyclic, and in addition can be benzocondensed in each case.

For example, there can be mentioned:

Thienyl, furanyl, pyrrolyl, oxazolyl, imidazolyl, pyrazolyl, isoxazolyl, isothiazolyl, oxadiazolyl, triazolyl, thiadiazolyl, etc. and benzo derivatives thereof, such as, e.g., benzofuranyl, benzothienyl, benzooxazolyl, benzimidazolyl, indazolyl, indolyl, isoindolyl, etc; or pyridazinyl, pyrimidinyl, pyrazinyl, triazinyl, etc., and benzo derivatives thereof, such as, e.g., quinolyl, isoquinolyl, etc; or azocinyl, indolizinyl, purinyl, etc., and benzo derivatives thereof, or quinolinyl, isoquinolinyl, cinnolinyl, phthalazinyl, quinazolinyl, quinoxalinyl, naphthyridinyl, pteridinyl, carbazolyl, acridinyl, phenazinyl, phenothiazinyl, phenoxazinyl, xanthenyl, oxepinyl, etc.

The heteroaryl radical can be benzocondensed in each case. For example, thiophene, furan, oxazole, thiazole, imidazole, pyrazole and benzo derivatives thereof can be mentioned as 5-ring heteroaromatic compounds, and pyridine, pyrimidine, triazine, quinoline, isoquinoline and benzo derivatives can be mentioned as 6-ring heteroaromatic compounds.

Heteroatoms are defined as oxygen, nitrogen or sulfur atoms.

If an acid group is included, the physiologically compatible salts of organic and inorganic bases, such as, for example, the readily soluble alkali and alkaline-earth salts, as well as N-methyl-glucamine, dimethyl-glucamine, ethyl-glucamine, lysine, 1,6-hexadiamine, ethanolamine, glucosamine, sarcosine, serinol, tris-hydroxy-methyl-amino-methane, aminopropanediol, Sovak base, and 1-amino-2,3,4-butanetriol, are suitable.

If a basic group is included, the physiologically compatible salts of organic and inorganic acids, such as hydrochloric acid, sulfuric acid, phosphoric acid, citric acid, tartaric acid, i.a., are suitable.

Especially preferred are those compounds of general formula (I) in which
R¹ stands for hydrogen, halogen, CF₃, C₃-C₆-cycloalkyl, which optionally is polysaturated and optionally is polysubstituted, or for the group C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl, C₁-C₆-aryl-C₁-C₆-alkyl or CF₃, in which C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl or C₁-C₆-aryl-C₁-C₆-alkyl optionally can be interrupted in one or more places, in the same way or differently, by oxygen, sulfur or nitrogen, or for the group sulfonyl-C₁-C₆-alkyl, sulfonamide, or cyano,
R² stands for halogen, CF₃, or C₃-C₆-cycloalkyl, which optionally is polysaturated and optionally is polysubstituted, or for the group C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl, C₁-C₆-aryl-C₁-C₆-alkyl or CF₃, in which C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl or C₁-C₆-aryl-C₁-C₆-alkyl optionally can be interrupted in one or more places, in the same way or differently, by oxygen, sulfur or nitrogen, or for the group sulfonyl-C₁-C₆-alkyl, sulfonamide, or cyano,
R³ stands for C₆-C₁₂-aryl, which optionally can be substituted in one or more places, in the same way or differently, with halogen, with C₁-C₆-alkyl, C₁-C₃-acyl, C₁-C₃-alkoxy, cyano, hydroxy, N—(CH₃)₂, CO₂—(C₁-C₃-alkyl), CO—NR⁴R⁵ or with CF₃, C₅-C₁₂-heteroaryl, which optionally can be substituted in one or more places, in the same way or differently, with chlorine and/or fluorine, with C₁-C₆-alkyl, C₁-C₃-acyl, C₁-C₃-alkoxy, cyano, hydroxy, N—(CH₃)₂, CO₂—(C₁-C₃-alkyl), CO—NR⁴R⁵ or with CF₃, C₃-C₆-cycloalkyl, which optionally can be substituted in one or more places, in the same way or differently, with chlorine and/or fluorine, CF₃, cyano, C₁-C₃-alkyl, C₁-C₃-acyl, hydroxy, N—(CH₃)₂, CO₂—(C₁-C₃-alkyl), CO—NR⁴R⁵ or C₁-C₃-alkoxy,
R⁴ stands for hydrogen, C₃-C₆-cycloalkyl, which optionally is substituted in one or more places, in the same way or differently, with C₁-C₃-alkyl, C₁-C₃-acyl, C₁-C₃-alkoxy or CF₃, for C₆-C₁₂-aryl, which optionally is substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₅-C₁₂-heteroaryl, which optionally is substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₁-C₆-alkyl, which can be substituted in any way desired,
R⁵ stands for hydrogen, C₃-C₆-cycloalkyl, which optionally is substituted in one or more places, in the same way or differently, with C₁-C₃-alkyl, C₁-C₃-acyl, C₁-C₃-alkoxy or CF₃, for C₆-C₁₂-aryl, which optionally is substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₅-C₁₂-heteroaryl, which optionally is substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₁-C₆-alkyl, which can be substituted in any way desired,
R⁴ and R⁵ together form a 5- to 8-membered ring, which can contain additional heteroatoms,
X stands for the groups sulfonyl, (CH₂)ₙ or carbonyl,
Y stands for carbonyl or (CH₂)ₙ,
Z stands for nitrogen, and
n stands for 0-2,
   as well as their isomers, diastereomers, enantiomers and salts.

Those compounds of general formula I, in which
R¹ stands for hydrogen,
R² stands for tert-butyl, cyano, bromine, or for the group —O—CF₃, —SO₂—CH₃ and is in para-position,
R³ stands for the group 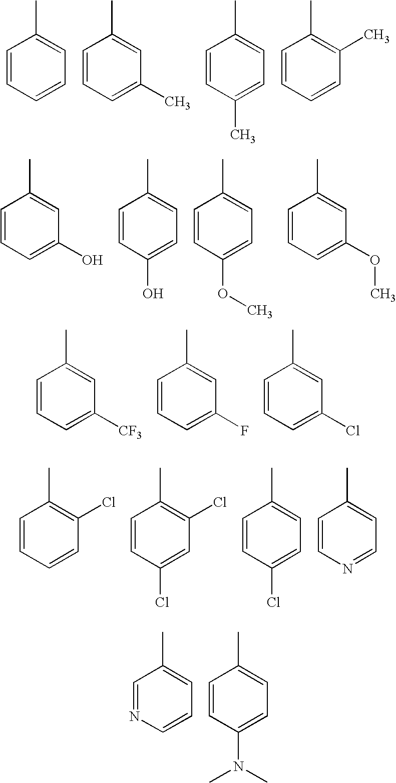
R⁴ stands for hydrogen or for the group —(CH₂)ₙ—N—(CH₃)₂, —(CH₂)₂—CH₃, —(CH₂)₂—NH—COCH₃, —(CH₂)—CHCH₃—OH, —(CH₂)₂—O—CH₃, —(CH₂)₂—OH, —CHCH₃—CH₂—OH, 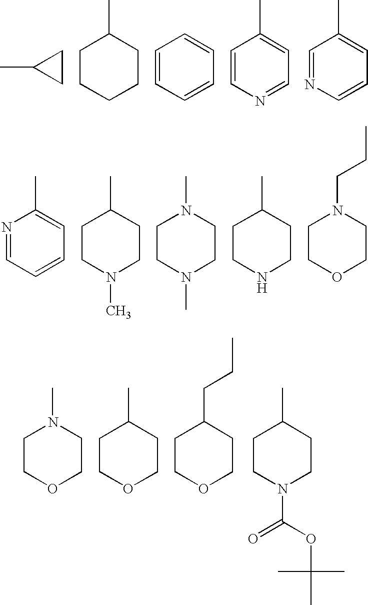
R⁵ stands for hydrogen,
X stands for sulfonyl, carbonyl or for the group CH₂,
Y stands for carbonyl or for the group (CH₂)ₙ,
Z stands for nitrogen or for 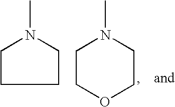
n is 1-2,
   as well as their isomers, diastereomers, enantiomers and salts,
   are quite especially preferred.

Also preferred are those compounds of general formula I in which
R¹ stands for hydrogen, tert-butyl, cyano, bromine, or for the group —O—CF₃, or —SO₂—CH₃,
R² stands for tert-butyl, cyano, bromine, or for the group —O—CF₃ or —SO₂—CH₃, and
R³ stands for C₆-C₁₂-aryl, which optionally can be substituted in one or more places, in the same way or differently, with halogen, with C₁-C₆-alkyl, C₁-C₃-acyl, C₁-C₃-alkoxy, cyano, hydroxy, N—(CH₃)₂, CO₂—(C₁-C₃-alkyl), CO—NR⁴R⁵ or with CF₃, C₅-C₁₂-heteroaryl, which optionally can be substituted in one or more places, in the same way or differently, with chlorine and/or fluorine, with C₁-C₆-alkyl, C₁-C₃-acyl, C₁-C₃-alkoxy, cyano, hydroxy, N—(CH₃)₂, CO₂—(C₁-C₃-alkyl), CO—NR⁴R⁵ or with CF₃, C₃-C₆-cycloalkyl, which optionally can be substituted in one or more places, in the same way or differently, with chlorine and/or fluorine, CF₃, cyano, C₁-C₃-alkyl, C₁-C₃-acyl, hydroxy, N—(CH₃)₂, CO₂—(C₁-C₃-alkyl), CO—NR⁴R⁵ or C₁-C₃-alkoxy,
R⁴ stands for hydrogen, C₃-C₆-cycloalkyl, which optionally is substituted in one or more places, in the same way or differently, with C₁-C₃-alkyl, C₁-C₃-acyl, C₁-C₃-alkoxy or CF₃, for C₆-C₁₂-aryl, which optionally is substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₅-C₁₂-heteroaryl, which optionally is substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₁-C₆-alkyl, which can be substituted in any way desired,
R⁵ stands for hydrogen, C₃-C₆-cycloalkyl, which optionally is substituted in one or more places, in the same way or differently, with C₁-C₃-alkyl, C₁-C₃-acyl, C₁-C₃-alkoxy or CF₃, for C₆-C₁₂-aryl, which optionally is substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₅-C₁₂-heteroaryl, which optionally is substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₁-C₆-alkyl, which can be substituted in any way desired,
R⁴ and R⁵ together form a 5- to 8-membered ring, which can contain additional heteroatoms,
X stands for the groups sulfonyl, (CH₂)ₙ or carbonyl,
Y stands for carbonyl or (CH₂)ₙ,
Z stands for nitrogen, and
n stands for 0-2,
   as well as their isomers, diastereomers, enantiomers and salts.

Also preferred are those compounds of general formula I in which
R¹ stands for hydrogen,
R² stands for tert-butyl, cyano, bromine, or for the group —O—CF₃ or —SO₂—CH₃ and is in para-position, and
R³ stands for C₆-C₁₂-aryl, which optionally can be substituted in one or more places, in the same way or differently, with halogen, with C₁-C₆-alkyl, C₁-C₃-acyl, C₁-C₃-alkoxy, cyano, hydroxy, N—(CH₃)₂, CO₂—(C₁-C₃-alkyl), CO—NR⁴R⁵ or with CF₃, C₅-C₁₂-heteroaryl, which optionally can be substituted in one or more places, in the same way or differently, with chlorine and/or fluorine, with C₁-C₆-alkyl, C₁-C₃-acyl, C₁-C₃-alkoxy, cyano, hydroxy, N—(CH₃)₂, CO₂—(C₁-C₃-alkyl), CO—NR⁴R⁵ or with CF₃, C₃-C₆-cycloalkyl, which optionally can be substituted in one or more places, in the same way or differently, with chlorine and/or fluorine, CF₃, cyano, C₁-C₃-alkyl, C₁-C₃-acyl, hydroxy, N—(CH₃)₂, CO₂—(C₁-C₃-alkyl), CO—NR⁴R⁵ or C₁-C₃-alkoxy,
R⁴ stands for hydrogen, C₃-C₆-cycloalkyl, which optionally is substituted in one or more places, in the same way or differently, with C₁-C₃-alkyl, C₁-C₃-acyl, C₁-C₃-alkoxy or CF₃, for C₆-C₁₂-aryl, which optionally is substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₅-C₁₂-heteroaryl, which optionally is substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₁-C₆-alkyl, which can be substituted in any way desired,
R⁵ stands for hydrogen, C₃-C₆-cycloalkyl, which optionally is substituted in one or more places, in the same way or differently, with C₁-C₃-alkyl, C₁-C₃-acyl, C₁-C₃-alkoxy or CF₃, for C₆-C₁₂-aryl, which optionally is substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₅-C₁₂-heteroaryl, which optionally is substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₁-C₆-alkyl, which can be substituted in any way desired,
R⁴ and R⁵ together form a 5- to 8-membered ring, which can contain additional heteroatoms,
X stands for the groups sulfonyl, (CH₂)ₙ or carbonyl,
Y stands for carbonyl or (CH₂)ₙ,
Z stands for nitrogen, and
n stands for 0-2,
   as well as their isomers, diastereomers, enantiomers and salts.

Also preferred are those compounds of general formula I in which
R¹ stands for hydrogen, halogen, CF₃, C₃-C₆-cycloalkyl, which optionally is polysaturated and optionally is polysubstituted, or for the group C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl, C₁-C₆-aryl-C₁-C₆-alkyl or CF₃, in which C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl or C₁-C₆-aryl-C₁-C₆-alkyl optionally can be interrupted in one or more places, in the same way or differently, by oxygen, sulfur or nitrogen, or for the group sulfonyl-C₁-C₆-alkyl, sulfonamide, or cyano,
R² stands for halogen, CF₃, C₃-C₆-cycloalkyl, which optionally is polysaturated and optionally is polysubstituted, or for the group C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl, C₁-C₆-aryl-C₁-C₆-alkyl or CF₃, in which C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl or C₁-C₆-aryl-C₁-C₆-alkyl optionally can be interrupted in one or more places, in the same way or differently, by oxygen, sulfur or nitrogen, or for the group sulfonyl-C₁-C₆-alkyl, sulfonamide, or cyano,
R³ stands for C₆-C₁₂-aryl, which optionally can be substituted in one or more places, in the same way or differently, with halogen, with C₁-C₆-alkyl, C₁-C₃-acyl, C₁-C₃-alkoxy, cyano, hydroxy, N—(CH₃)₂, CO₂—(C₁-C₃-alkyl), CO—NR⁴R⁵ or with CF₃, C₅-C₁₂-heteroaryl, which optionally can be substituted in one or more places, in the same way or differently, with chlorine and/or fluorine, with C₁-C₆-alkyl, C₁-C₃-acyl, C₁-C₃-alkoxy, cyano, hydroxy, N—(CH₃)₂, CO₂—(C₁-C₃-alkyl), CO—NR⁴R⁵ or with CF₃, C₃-C₆-cycloalkyl, which optionally can be substituted in one or more places, in the same way or differently, with chlorine and/or fluorine, CF₃, cyano, C₁-C₃-alkyl, C₁-C₃-acyl, hydroxy, N—(CH₃)₂, CO₂—(C₁-C₃-alkyl), CO—NR⁴R⁵ or C₁-C₃-alkoxy,
R⁴ stands for hydrogen, C₃-C₆-cycloalkyl, which optionally is substituted in one or more places, in the same way or differently, with C₁-C₆-alkyl, C₁-C₆-acyl, C₁-C₆-alkoxy or CF₃, for C₆-C₁₂-aryl, which optionally is substituted in one or more places, in the same way or differently, with halogen, with C₁-C₆-alkyl, C₁-C₆-acyl, C₁-C₆-alkoxy, N—C₁-C₆-alkyl-C₁-C₆-alkyl, CF₃ or cyano, or for C₅-C₁₂-heteroaryl, which optionally is substituted in one or more places, in the same way or differently, with halogen, with C₁-C₆-alkyl, C₁-C₆-acyl, C₁-C₆-alkoxy, N—C₁-C₆-alkyl-C₁-C₆-alkyl, CF₃ or cyano, or for C₁-C₆-alkyl, which can be substituted in any way desired,
R⁵ stands for hydrogen, C₃-C₆-cycloalkyl, which optionally is substituted in one or more places, in the same way or differently, with C₁-C₆-alkyl, C₁-C₆-acyl, C₁-C₆-alkoxy or CF₃, for C₆-C₁₂-aryl, which optionally is substituted in one or more places, in the same way or differently, with halogen, with C₁-C₆-alkyl, C₁-C₆-acyl, C₁-C₆-alkoxy, N—C₁-C₆-alkyl-C₁-C₆-alkyl, CF₃ or cyano, or for C₅-C₁₂-heteroaryl, which optionally is substituted in one or more places, in the same way or differently, with halogen, with C₁-C₆-alkyl, C₁-C₆-acyl, C₁-C₆-alkoxy, N—C₁-C₆-alkyl-C₁-C₆-alkyl, CF₃ or cyano, or for C₁-C₆-alkyl, which can be substituted in any way desired,
R⁴ and R⁵ together form a 5- to 8-membered ring, which can contain additional heteroatoms, and
X stands for the groups sulfonyl, (CH₂)ₙ or carbonyl,
Y stands for carbonyl or (CH₂)ₙ,
Z stands for nitrogen,
n stands for 0-4,
   as well as their isomers, diastereomers, enantiomers and salts.

Also preferred are those compounds of general formula I in which
R¹ stands for hydrogen,
R² stands for tert-butyl, cyano, bromine, or for the group —O—CF₃ or —SO₂—CH₃ and is in para-position, and
R³ stands for C₆-C₁₂-aryl, which optionally can be substituted in one or more places, in the same way or differently, with halogen, with C₁-C₆-alkyl, C₁-C₃-acyl, C₁-C₃-alkoxy, cyano, hydroxy, N—(CH₃)₂, CO₂—(C₁-C₃-alkyl), CO—NR⁴R⁵ or with CF₃, C₅-C₁₂-heteroaryl, which optionally can be substituted in one or more places, in the same way or differently, with chlorine and/or fluorine, with C₁-C₆-alkyl, C₁-C₃-acyl, C₁-C₃-alkoxy, cyano, hydroxy, N—(CH₃)₂, CO₂—(C₁-C₃-alkyl), CO—NR⁴R⁵ or with CF₃, C₃-C₆-cycloalkyl, which optionally can be substituted in one or more places, in the same way or differently, with chlorine and/or fluorine, CF₃, cyano, C₁-C₃-alkyl, C₁-C₃-acyl, hydroxy, N—(CH₃)₂, CO₂—(C₁-C₃-alkyl), CO—NR⁴R⁵ or C₁-C₃-alkoxy,
R⁴ stands for hydrogen or for the group —(CH₂)ₙ—N—(CH₃)₂, —(CH₂)₂—CH₃, —(CH₂)₂—NH—COCH₃, —(CH₂)—CHCH₃—OH, —(CH₂)₂—O—CH₃, —(CH₂)₂—OH, —CHCH₃—CH₂—OH,

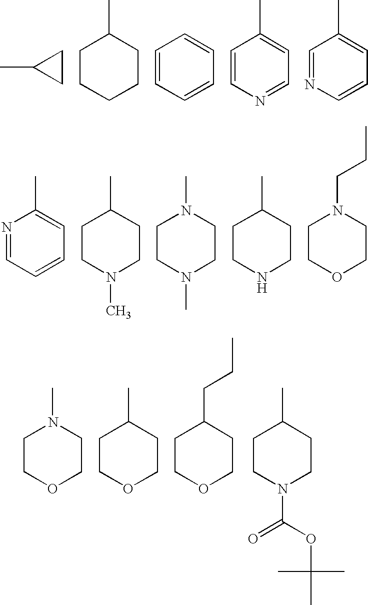

R⁵ stands for hydrogen,

X stands for sulfonyl, carbonyl or for the group CH₂,

Y stands for carbonyl or for the group (CH₂)ₙ,

Z stands for nitrogen or for

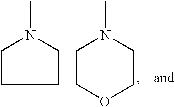

n stands for 1-2,

as well as their isomers, diastereomers, enantiomers and salts.

Also preferred are those compounds of general formula I in which

R¹ stands for hydrogen, halogen, CF₃, C₃-C₆-cycloalkyl, which optionally is polysaturated and optionally is polysubstituted, or for the group C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl, C₁-C₆-aryl-C₁-C₆-alkyl or CF₃, in which C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl or C₁-C₆-aryl-C₁-C₆-alkyl optionally can be interrupted in one or more places, in the same way or differently, by oxygen, sulfur or nitrogen, or for the group sulfonyl-C₁-C₆-alkyl, sulfonamide, or cyano,
R² stands for halogen, CF₃, C₃-C₆-cycloalkyl, which optionally is polysaturated and optionally is polysubstituted, or for the group C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl, C₁-C₆-aryl-C₁-C₆-alkyl or CF₃, in which C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl or C₁-C₆-aryl-C₁-C₆-alkyl optionally can be interrupted in one or more places, in the same way or differently, by oxygen, sulfur or nitrogen, or for the group sulfonyl-C₁-C₆-alkyl, sulfonamide, or cyano,
R³ stands for the group 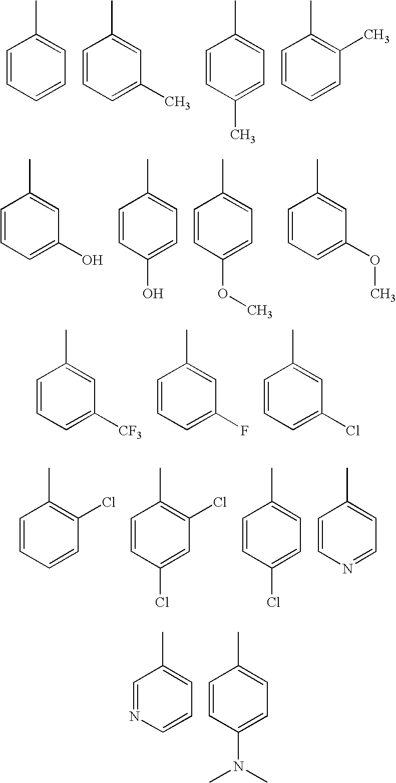
R⁴ stands for hydrogen, C₃-C₆-cycloalkyl, which optionally is substituted in one or more places, in the same way or differently, with C₁-C₃-alkyl, C₁-C₃-acyl, C₁-C₃-alkoxy or CF₃, for C₆-C₁₂-aryl, which optionally is substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₅-C₁₂-heteroaryl, which optionally is substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₁-C₆-alkyl, which can be substituted in any way desired,
R⁵ stands for hydrogen, C₃-C₆-cycloalkyl, which optionally is substituted in one or more places, in the same way or differently, with C₁-C₃-alkyl, C₁-C₃-acyl, C₁-C₃-alkoxy or CF₃, for C₆-C₁₂-aryl, which optionally is substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₅-C₁₂-heteroaryl, which optionally is substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₁-C₆-alkyl, which can be substituted in any way desired,
R⁴ and R⁵ together form a 5- to 8-membered ring, which can contain additional heteroatoms,
X stands for the groups sulfonyl, (CH₂)ₙ or carbonyl,
Y stands for carbonyl or (CH₂)ₙ,
Z stands for nitrogen, and
n stands for 0-2,
   as well as their isomers, diastereomers, enantiomers and salts.

Also preferred are those compounds of general formula I in which
R¹ stands for hydrogen,
R² stands for tert-butyl, cyano, bromine or for the group —O—CF₃ or —SO₂—CH₃ and is in para-position, and
R³ stands for the group 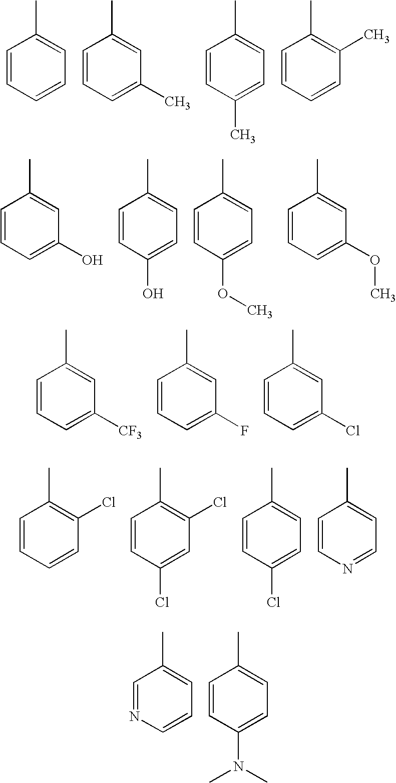
R⁴ stands for hydrogen, C₃-C₆-cycloalkyl, which optionally is substituted in one or more places, in the same way or differently, with C₁-C₃-alkyl, C₁-C₃-acyl, C₁-C₃-alkoxy or CF₃, for C₆-C₁₂-aryl, which optionally is substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₅-C₁₂-heteroaryl, which optionally is substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₁-C₆-alkyl, which can be substituted in any way desired,
R⁵ stands for hydrogen, C₃-C₆-cycloalkyl, which optionally is substituted in one or more places, in the same way or differently, with C₁-C₃-alkyl, C₁-C₃-acyl, C₁-C₃-alkoxy or CF₃, for C₆-C₁₂-aryl, which optionally is substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₅-C₁₂-heteroaryl, which optionally is substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₁-C₆-alkyl, which can be substituted in any way desired,
X stands for sulfonyl, carbonyl or for the group CH₂,
Y stands for carbonyl or for the group (CH₂)ₙ,
Z stands for nitrogen or for

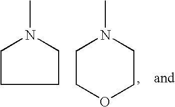

n stands for 1-2,

as well as their isomers, diastereomers, enantiomers, and salts.

Also preferred are those compounds of general formula I in which
R¹ stands for hydrogen, halogen, CF₃, C₃-C₆-cycloalkyl, which optionally is polysaturated and optionally is polysubstituted, or for the group C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl, C₁-C₆-aryl-C₁-C₆-alkyl or CF₃, in which C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl or C₁-C₆-aryl-C₁-C₆-alkyl optionally can be interrupted in one or more places, in the same way or differently, by oxygen, sulfur or nitrogen, or for the group sulfonyl-C₁-C₆-alkyl, sulfonamide, or cyano,
R² is halogen, CF₃, C₃-C₆-cycloalkyl, which optionally is polysaturated and optionally is polysubstituted, or for the group C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl, C₁-C₆-aryl-C₁-C₆-alkyl or CF₃, in which C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl or C₁-C₆-aryl-C₁-C₆-alkyl optionally can be interrupted in one or more places, in the same way or differently, by oxygen, sulfur or nitrogen, or for the group sulfonyl-C₁-C₆-alkyl, sulfonamide, or cyano,
R³ stands for C₆-C₁₂-aryl, which optionally can be substituted in one or more places, in the same way or differently, with halogen, with C₁-C₆-alkyl, C₁-C₃-acyl, C₁-C₃-alkoxy, cyano, hydroxy, N—(CH₃)₂, CO₂—(C₁-C₃-alkyl), CO—NR⁴R⁵ or with CF₃, C₅-C₁₂-heteroaryl, which optionally can be substituted in one or more places, in the same way or differently, with chlorine and/or fluorine, with C₁-C₆-alkyl, C₁-C₃-acyl, C₁-C₃-alkoxy, cyano, hydroxy, N—(CH₃)₂, CO₂—(C₁-C₃-alkyl), CO—NR⁴R⁵ or with CF₃, C₃-C₆-cycloalkyl, which optionally can be substituted in one or more places, in the same way or differently, with chlorine and/or fluorine, CF₃, cyano, C₁-C₃-alkyl, C₁-C₃-acyl, hydroxy, N—(CH₃)₂, CO₂—(C₁-C₃-alkyl), CO—NR⁴R⁵ or C₁-C₃-alkoxy,
R⁴ stands for hydrogen or for the group —(CH₂)ₙ—N—(CH₃)₂, —(CH₂)₂—CH₃, —(CH₂)₂—NH—COCH₃, —(CH₂)—CHCH₃—OH, —(CH₂)₂—O—CH₃, —(CH₂)₂—OH, —CHCH₃—CH₂—OH, 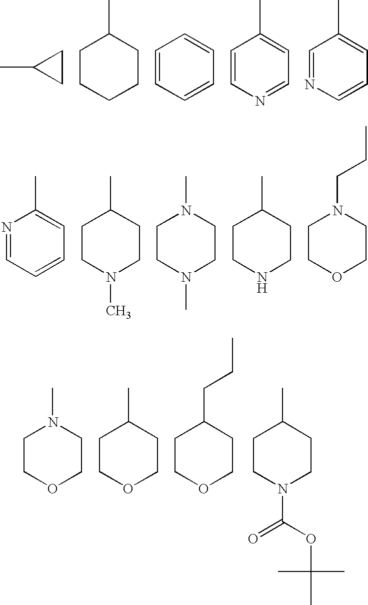
R⁵ stands for hydrogen,
X stands for the groups sulfonyl, (CH₂)ₙ or carbonyl,
Y stands for carbonyl or (CH₂)ₙ,
Z stands for nitrogen, and
n stands for 0-2,
   as well as their isomers, diastereomers, enantiomers, and salts.

Also preferred are those compounds of general formula in which
R¹ stands for hydrogen, halogen, CF₃, C₃-C₆-cycloalkyl, which optionally is polysaturated and optionally is polysubstituted, or for the group C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl, C₁-C₆-aryl-C₁-C₆-alkyl or CF₃, in which C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl or C₁-C₆-aryl-C₁-C₆-alkyl optionally can be interrupted in one or more places, in the same way or differently, by oxygen, sulfur or nitrogen, or for the group sulfonyl-C₁-C₆-alkyl, sulfonamide, or cyano,
R² is halogen, CF₃, C₃-C₆-cycloalkyl, which optionally is polysaturated and optionally is polysubstituted, or for the group C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl, C₁-C₆-aryl-C₁-C₆-alkyl or CF₃, in which C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl or C₁-C₆-aryl-C₁-C₆-alkyl optionally can be interrupted in one or more places, in the same way or differently, by oxygen, sulfur or nitrogen, or for the group sulfonyl-C₁-C₆-alkyl, sulfonamide, or cyano,
R³ stands for C₆-C₁₂-aryl, which optionally can be substituted in one or more places, in the same way or differently, with halogen, with C₁-C₆-alkyl, C₁-C₃-acyl, C₁-C₃-alkoxy, cyano, hydroxy, N—(CH₃)₂, CO₂—(C₁-C₃-alkyl), CO—NR⁴R⁵ or with CF₃, C₅-C₁₂-heteroaryl, which optionally can be substituted in one or more places, in the same way or differently, with chlorine and/or fluorine, with C₁-C₆-alkyl, C₁-C₃-acyl, C₁-C₃-alkoxy, cyano, hydroxy, N—(CH₃)₂, CO₂—(C₁-C₃-alkyl), CO—NR⁴R⁵ or with CF₃, C₃-C₆-cycloalkyl, which optionally can be substituted in one or more places, in the same way or differently, with chlorine and/or fluorine, CF₃, cyano, C₁-C₃-alkyl, C₁-C₃-acyl, hydroxy, N—(CH₃)₂, CO₂—(C₁-C₃-alkyl), CO—NR⁴R⁵ or C₁-C₃-alkoxy,
R⁴ stands for hydrogen, C₃-C₆-cycloalkyl, which optionally is substituted in one or more places, in the same way or differently, with C₁-C₃-alkyl, C₁-C₃-acyl, C₁-C₃-alkoxy or CF₃, for C₆-C₁₂-aryl, which optionally is substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₅-C₁₂-heteroaryl, which optionally is substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₁-C₆-alkyl, which can be substituted in any way desired,
R⁵ stands for hydrogen, C₃-C₆-cycloalkyl, which optionally is substituted in one or more places, in the same way or differently, with C₁-C₃-alkyl, C₁-C₃-acyl, C₁-C₃-alkoxy or CF₃, for C₆-C₁₂-aryl, which optionally is substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₅-C₁₂-heteroaryl, which optionally is substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₁-C₆-alkyl, which can be substituted in any way desired,
R⁴ and R⁵ together form a 5- to 8-membered ring that can contain additional heteroatoms,
X stands for the groups sulfonyl, (CH₂)ₙ or carbonyl,
Y stands for carbonyl or (CH₂)ₙ,
Z stands for nitrogen or for 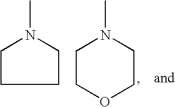
n stands for 0-2,
   as well as their isomers, diastereomers, enantiomers and salts.

The following compounds corresponding to this invention are quite especially preferred:
5-(4-tert-Butylbenzenesulfonylamino)-3-phenyl-1H-indole-2-carboxylic acid-cyclopropylamide
5-(4-tert-Butylbenzenesulfonylamino)-3-phenyl-1H-indole-2-carboxylic acid-pyridin-3-yl amide
5-(4-tert-Butylbenzenesulfonylamino)-3-phenyl-1H-indole-2-carboxylic acid-cyclohexylamide
4-tert-Butyl-N-[3-phenyl-2-(pyrrolidine-1-carbonyl)-1H-indol-5-yl]-benzenesulfonamide
4-tert-Butyl-N-[2-(morpholine-4-carbonyl)-3-phenyl-1H-indol-5yl]-benzene-sulfonamide
5-(4-tert-Butylbenzenesulfonylamino)-3-phenyl-1H-indole-2-carboxylic acid-(2-dimethylaminoethyl)amide
5-(4-Cyanobenzenesulfonylamino)-3-phenyl-1H-indole-2-carboxylic acid-propylamide
5-(4-Bromobenzenesulfonylamino)-3-phenyl-1H-indole-2-carboxylic acid-propylamide
5-(4-tert-Butylbenzenesulfonylamino)-3-phenyl-1H-indole-2-carboxylic acid-propylamide
5-(4-(Trifluoromethoxy)benzenesulfonylamino)-3-phenyl-1H-indole-2-carboxylic acid-propylamide
5-(4-(Methylsulfonyl)benzenesulfonylamino)-3-phenyl-1H-indole-2-carboxylic acid-propylamide
5-(4-tert-Butylbenzenesulfonylamino)-3-phenyl-1H-indole-2-carboxylic acid-phenylamide
5-(4-Cyanobenzenesulfonylamino)-3-phenyl-1H-indole-2-carboxylic acid-phenylamide
5-(4-Bromobenzenesulfonylamino)-3-phenyl-1H-indole-2-carboxylic acid-phenylamide
5-(4-(Trifluoromethoxy)benzenesulfonylamino)-3-phenyl-1H-indole-2-carboxylic acid-phenylamide
5-(4-(Methylsulfonyl)benzenesulfonylamino)-3-phenyl-1H-indole-2-carboxylic acid-phenylamide
5-(4-tert-Butylbenzenesulfonylamino)-3-phenyl-1H-indole-2-carboxylic acid-pyridin-2-ylamide
5-(4-Cyanobenzenesulfonylamino)-3-phenyl-1H-indole-2-carboxylic acid-pyridin-2-ylamide
5-(4-tert-Butylbenzenesulfonylamino)-3-phenyl-1H-indole-2-carboxylic acid-(2-morpholin-4-ylethyl)amide
5-(4-tert-Butylbenzenesulfonylamino)-3-phenyl-1H-indole-2-carboxylic acid-(4-methylpiperazin-1-yl)amide
5-(4-tert-Butylbenzenesulfonylamino)-1H-indole-2-carboxylic acid pyrindin-4-ylamide
5-(4-tert-Butylbenzenesulfonylamino)-1H-indole
5-(4-tert-Butylbenzenesulfonylamino)-3-phenyl-1H-indole-2-carboxylic acid-pyrindin-4-ylamide
5-(4-tert-Butylbenzylamino)-3-phenyl-1H-indole-2-carboxylic acid pyridin-4-ylamide
5-(4-tert-Butylbenzoylamino)-3-phenyl-1H-indole-2-carboxylic acid-(2-dimethylaminoethyl)amide
5-(4-tert-Butylbenzenesulfonylamino)-3-(2-chlorophenyl)-1H-indole-2-carboxylic acid-(2-dimethylaminoethyl)amide
5-(4-tert-Butylbenzenesulfonylamino)-3-(3-chlorophenyl)-1H-indole-2-carboxylic acid-(2-dimethylaminoethyl)amide
5-(4-tert-Butylbenzenesulfonylamino)-3-(4-chlorophenyl)-1H-indole-2-carboxylic acid-(2-dimethylaminoethyl)amide
5-(4-tert-Butylbenzenesulfonylamino)-3-(2,4-dichlorophenyl)-1H-indole-2-carboxylic acid-(2-dimethylaminoethyl)amide
5-(4-tert-Butylbenzenesulfonylamino)-3-(2-methylphenyl)-1H-indole-2-carboxylic acid-(2-dimethylaminoethyl)amide
5-(4-tert-Butylbenzenesulfonylamino)-3-(4-methylphenyl)-1H-indole-2-carboxylic acid-(2-dimethylaminoethyl)amide
5-(4-tert-Butylbenzenesulfonylamino)-3-(4-methylphenyl)-1H-indole-2-carboxylic acid pyridin-4ylamide
5-(4-tert-Butylbenzenesulfonylamino)-3-(4-methoxyphenyl)-1H-indole-2-carboxylic acid-(2-dimethylaminoethyl)amide
5-(4-tert-Butylbenzenesulfonylamino)-3-pyridin-3-yl-1H-indole-2-carboxylic acid-(2-dimethylaminoethyl)amide
4-tert-Butyl-N-(3-phenyl-1H-indol-5yl)-benzenesulfonamide
5-(4-tert-Butylbenzenesulfonylamino)-3-(4-hydroxyphenyl)-1H-indole-2-carboxylic acid-(2-dimethylaminoethyl)amide
5-(4-tert-Butylbenzenesulfonylamino)-3-(3-fluorophenyl)-1H-indole-2-carboxylic acid-(2-dimethylaminoethyl)amide
5-(4-tert-Butylbenzenesulfonylamino)-3-phenyl-1H-indole-2-carboxylic acid-(2-hydroxypropyl)amide
5-(4-tert-Butylbenzenesulfonylamino)-3-phenyl-1H-indole-2-carboxylic acid-(2-methoxyethyl)amide
5-(4-tert-Butylbenzenesulfonylamino)-3-phenyl-1H-indole-2-carboxylic acid-(2-hydroxyethyl)amide
5-(4-tert-Butylbenzenesulfonylamino)-3-phenyl-1H-indole-2-carboxylic acid-(2-hydroxy-1-methylethyl)amide
5-(4-tert-Butylbenzenesulfonylamino)-3-phenyl-1H-indole-2-carboxylic acid-(2-acetylaminoethyl)amide
5-(4-tert-Butylbenzenesulfonylamino)-3-phenyl-1H-indole-2-carboxylic acid-(tetrahydropyran-4-yl)amide
5-(4-tert-Butylbenzenesulfonylamino)-3-phenyl-1H-indole-2-carboxylic acid-(1-methylpiperidin-4-yl)amide
5-(4-tert-Butylbenzenesulfonylamino)-3-(4-N,N-dimethylamino-phenyl)-1H-indole-2-carboxylic acid-(2-dimethylaminoethyl)amide
5-(4-tert-Butylbenzenesulfonylamino)-3-(3-methoxyphenyl-1H-indole-2-carboxylic acid-(2-dimethylaminoethyl)amide
5-(4-tert-Butylbenzenesulfonylamino)-3-(3-trifluoromethylphenyl)-1H-indole-2-carboxylic acid-(2-dimethylaminoethyl)amide
5-(4-tert-Butylbenzenesulfonylamino)-3-(3-fluorophenyl)-1H-indole-2-carboxylic acid-(2-hydroxyethyl)amide
5-(4-tert-Butylbenzenesulfonylamino)-3-(3-fluorophenyl)-1H-indole-2-carboxylic acid-(tetrahydropyran-4-yl)amide
5-(4-tert-Butylbenzenesulfonylamino)-3-(3-fluorophenyl)-1H-indole-2-carboxylic acid-(2-acetylaminoethyl)amide
5-(4-tert-Butylbenzenesulfonylamino)-3-(3-fluorophenyl)-1H-indole-2-carboxylic acid-(2-morpholin-4-ylethyl)amide
5-(4-tert-Butylbenzenesulfonylamino)-3-(3-methoxyphenyl)-1H-indole-2-carboxylic acid-(2-hydroxyethyl)amide
5-(4-tert-Butylbenzenesulfonylamino)-3-(3-methoxyphenyl)-1H-indole-2-carboxylic acid-(2-acetylaminoethyl)amide
5-(4-tert-Butylbenzenesulfonylamino)-3-pyridin-3-yl-1H-indole-2-carboxylic acid-(2-acetylaminoethyl)amide
5-(4-tert-Butylbenzenesulfonylamino)-3-pyridin-3-yl-1H-indole-2-carboxylic acid-(tetrahydropyran-4yl)amide
5-(4-tert-Butylbenzenesulfonylamino)-3-pyridin-4-yl-1H-indole-2-carboxylic acid-(2-acetylaminoethyl)amide
5-(4-tert-Butylbenzenesulfonylamino)-3-pyridin-4-yl-1H-indole-2-carboxylic acid-(2-morpholin-4-ylethyl)amide
5-(4-tert-Butylbenzenesulfonylamino)-3-pyridin-4-yl-1H-indole-2-carboxylic acid-(tetrahydropyran-4-yl)amide
5-(4-tert-Butylbenzenesulfonylamino)-3-(3-methylphenyl)-1H-indole-2-carboxylic acid-(2-acetylaminoethyl)amide
5-(4-tert-Butylbenzenesulfonylamino)-3-(3-methylphenyl)-1H-indole-2-carboxylic acid-(2-hydroxyethyl)amide
5-(4-tert-Butylbenzenesulfonylamino)-3-(3-methylphenyl)-1H-indole-2-carboxylic acid-(2-morpholin-4-ylethyl)amide
5-(4-tert-Butylbenzenesulfonylamino)-3-(3-methylphenyl)-1H-indole-2-carboxylic acid-(tetrahydropyran-4-yl)amide
5-(4-tert-Butylbenzenesulfonylamino)-3-pyridin-3-yl-1H-indole-2-carboxylic acid-(2-morpholin-4-ylethyl)amide
5-(4-tert-Butylbenzenesulfonylamino)-3-(3-methoxyphenyl)-1H-indole-2-carboxylic acid-(tetrahydropyran-4-yl)amide
5-(4-tert-Butylbenzenesulfonylamino)-3-(3-methoxyphenyl)-1H-indole-2-carboxylic acid-(2-morpholin-4-ylethyl)amide
5-(4-tert-Butylbenzenesulfonylamino)-3-phenyl-1H-indole-2-carboxylic acid piperidin-4-ylamide
4-{[5-(4-tert-Butylbenzenesulfonylamino)-3-phenyl-1H-indole-2-carbonyl]-piperidine-1-carboxylic acid-tert-butyl ester
5-(4-tert-Butyl-benzenesulfonyl-amino)-3-naphthalen-1-yl-1H-indole-2-carboxylic acid (2-morpholin-4-yl-ethyl)-amide
5-(4-tert-Butyl-benzenesulfonyl-amino)-3-m-tolyl-1H-indole-2-carboxylic acid (2-morpholin-4-yl-ethyl)-amide
5-(4-tert-Butyl-benzenesulfonyl-amino)-3-thiophen-2-yl-1H-indole-2-carboxylic acid (2-morpholin-4-yl-ethyl)-amide
5-(4-tert-Butyl-benzenesulfonyl-amino)-3-thiophen-3-yl-1H-indole-2-carb oxylic acid (2-morpholin-4-yl-ethyl)-amide
3-Benzofuran-2-yl-5-(4-tert-butyl-benzenesulfonylamino)-1H-indole-2-carboxylic acid (2-morpholin-4-yl-ethyl)-amide
5-(4-tert-Butyl-benzenesulfonyl-amino)-3-(5-chloro-thiophen-2-yl)-1H-indole-2-carboxylic acid (2-morpholin-4-yl-ethyl)-amide
5-(4-tert-Butyl-benzenesulfonyl-amino)-3-furan-2-yl-1H-indole-2-carboxylic acid (2-morpholin-4-yl-ethyl)-amide
5-(4-tert-Butyl-benzenesulfonyl-amino)-3-(3-fluoro-4-methoxy-phenyl)-1H-indole-2-carboxylic acid (2-morpholin-4-yl-ethyl)-amide
3-Benzo[1,3]dioxol-5-yl-5-(4-tert-butyl-benzenesulfonylamino)-1H-indole-2-carboxylic acid (2-morpholin-4-yl-ethyl)-amide
3-(4-Acetyl-phenyl)-5-(4-tert-butyl-benzenesulfonylamino)-1H-indole-2-carboxylic acid (2-morpholin-4-yl-ethyl)-amide
3-(3-Acetyl-phenyl)-5-(4-tert-butyl-benzenesulfonylamino)-1H-indole-2-carboxylic acid (2-morpholin-4-yl-ethyl)-amide
3-Benzo[b]thiophen-2-yl-5-(4-tert-butyl-benzenesulfonylamino)-1H-indole-2-carboxylic acid (2-morpholin-4-yl-ethyl)-amide
3-Benzo[b]thiophen-3-yl-5-(4-tert-butyl-benzenesulfonylamino)-1H-indole-2-carboxylic acid (2-morpholin-4-yl-ethyl)-amide
5-(4-tert-Butyl-benzenesulfonyl-amino)-3-(5-methyl-thiophen-2-yl)-1H-indole-2-carboxylic acid (2-morpholin-4-yl-ethyl)-amide
3-[5-(4-tert-Butyl-benzenesulfonyl-amino)-2-(2-morpholin-4-yl-ethylcarbamoyl)-1H-indol-3-yl]-benzoic acid methyl ester
5-(4-tert-Butyl-benzenesulfonyl-amino)-3-(2-fluoro-3-methoxy-phenyl)-1H-indole-2-carboxylic acid (2-morpholin-4-yl-ethyl)-amide
5-(4-tert-Butyl-benzenesulfonyl-amino)-3-(3-chloro-4-methyl-phenyl)-1H-indole-2-carboxylic acid (2-morpholin-4-yl-ethyl)-amide
5-(4-tert-Butyl-benzenesulfonyl-amino)-3-(2,4-dimethoxy-pyrimidin-5-yl)-1H-indole-2-carboxylic acid (2-morpholin-4-yl-ethyl)-amide
5-(4-tert-Butyl-benzenesulfonyl-amino)-3-(2,5-difluoro-phenyl)-1H-indole-2-carboxylic acid (2-morpholin-4-yl-ethyl)-amide
5-(4-tert-Butyl-benzenesulfonyl-amino)-3-(2,4-difluoro-phenyl)-1H-indole-2-carboxylic acid (2-morpholin-4-yl-ethyl)-amide
5-(4-tert-Butyl-benzenesulfonyl-amino)-3-(2,3-difluoro-phenyl)-1H-indole-2-carboxylic acid (2-morpholin-4-yl-ethyl)-amide
5-(4-tert-Butyl-benzenesulfonyl-amino)-3-(2,6-difluoro-phenyl)-1H-indole-2-carboxylic acid (2-morpholin-4-yl-ethyl)-amide
5-(4-tert-Butyl-benzenesulfonyl-amino)-3-(3-hydroxy-phenyl)-1H-indole-2-carboxylic acid (2-morpholin-4-yl-ethyl)-amide
5-(4-tert-Butyl-benzenesulfonyl-amino)-3-(4-hydroxy-phenyl)-1H-indole-2-carboxylic acid (2-morpholin-4-yl-ethyl)-amide
5-(4-tert-Butyl-benzenesulfonyl-amino)-3-(3-fluoro-4-methyl-phenyl)-1H-indole-2-carboxylic acid (2-morpholin-4-yl-ethyl)-amide
5-(4-tert-Butyl-benzenesulfonyl-amino)-3-(4-trifluoromethyl-phenyl)-1H-indole-2-carboxylic acid (2-morpholin-4-yl-ethyl)-amide
5-(4-tert-Butyl-benzenesulfonyl-amino)-3-(4-cyanomethyl-phenyl)-1H-indole-2-carboxylic acid (2-morpholin-4-yl-ethyl)-amide
5-(4-tert-Butyl-benzenesulfonyl-amino)-1H,1′H-[3,4′]biindolyl-2-carboxylic acid (2-morpholin-4-yl-ethyl)-amide
5-(4-tert-Butyl-benzenesulfonyl-amino)-3-(3-cyano-4-fluoro-phenyl)-1H-indole-2-carboxylic acid (2-morpholin-4-yl-ethyl)-amide
5-(4-tert-Butyl-benzenesulfonyl-amino)-3-(2-fluoro-phenyl)-1H-indole-2-carboxylic acid (2-morpholin-4-yl-ethyl)-amide
5-(4-tert-Butyl-benzenesulfonyl-amino)-3-(3,4-difluoro-phenyl)-1H-indole-2-carboxylic acid (2-morpholin-4-yl-ethyl)-amide
5-(4-tert-Butyl-benzenesulfonyl-amino)-3-(3-cyano-phenyl)-1H-indole-2-carboxylic acid (2-morpholin-4-yl-ethyl)-amide
5-(4-tert-Butyl-benzenesulfonyl-amino)-3-(4-cyano-phenyl)-1H-indole-2-carboxylic acid (2-morpholin-4-yl-ethyl)-amide
5-(4-tert-Butyl-benzenesulfonyl-amino)-3-(4-methyl-thiophen-2-yl)-1H-indole-2-carboxylic acid (2-morpholin-4-yl-ethyl)-amide
5-(4-tert-Butyl-benzenesulfonyl-amino)-3-phenyl-1H-indole-2-carboxylic acid (3-chloro-phenyl)-amide
5-(4-tert-Butyl-benzenesulfonyl-amino)-3-phenyl-1H-indole-2-carboxylic acid (3-methyl-isoxazol-5-yl)-amide
5-(4-tert-Butyl-benzenesulfonyl-amino)-3-phenyl-1H-indole-2-carboxylic acid (4-fluoro-phenyl)-amide
5-(4-tert-Butyl-benzenesulfonyl-amino)-3-phenyl-1H-indole-2-carboxylic acid (2-fluoro-phenyl)-amide
5-(4-tert-Butyl-benzenesulfonyl-amino)-3-phenyl-1H-indole-2-carboxylic acid (6-methyl-pyridin-2-yl)-amide
5-(4-tert-Butyl-benzenesulfonyl-amino)-3-phenyl-1H-indole-2-carboxylic acid (5-carbamoyl-pyridin-2-yl)-amide
5-(4-tert-Butyl-benzenesulfonyl-amino)-3-phenyl-1H-indole-2-carboxylic acid (4-hydroxy-phenyl)-amide
5-(4-tert-Butyl-benzenesulfonyl-amino)-3-phenyl-1H-indole-2-carboxylic acid (2-methoxy-phenyl)-amide
5-(4-tert-Butyl-benzenesulfonyl-amino)-3-phenyl-1H-indole-2-carboxylic acid (3-methoxy-phenyl)-amide
5-(4-tert-Butyl-benzenesulfonyl-amino)-3-phenyl-1H-indole-2-carboxylic acid (4-methoxy-phenyl)-amide
5-(4-tert-Butyl-benzenesulfonyl-amino)-3-phenyl-1H-indole-2-carboxylic acid (4-chloro-phenyl)-amide
5-(4-tert-Butyl-benzenesulfonyl-amino)-3-phenyl-1H-indole-2-carboxylic acid (4-dimethylamino-phenyl)-amide
5-(4-tert-Butyl-benzenesulfonyl-amino)-3-phenyl-1H-indole-2-carboxylic acid (5-chloro-pyridin-2-yl)-amide
5-(4-tert-Butyl-benzenesulfonyl-amino)-3-phenyl-1H-indole-2-carboxylic acid p-tolylamide
5-(4-tert-Butyl-benzenesulfonyl-amino)-3-phenyl-1H-indole-2-carboxylic acid pyrazin-2-ylamide
5-(4-tert-Butyl-benzenesulfonyl-amino)-3-phenyl-1H-indole-2-carboxylic acid (4-cyano-phenyl)-amide
5-(4-tert-Butyl-benzenesulfonyl-amino)-3-phenyl-1H-indole-2-carboxylic acid (3-methyl-isothiazol-5-yl)-amide
5-(4-tert-Butyl-benzenesulfonyl-amino)-3-phenyl-1H-indole-2-carboxylic acid (4-bromo-phenyl)-amide
5-(4-tert-Butyl-benzenesulfonyl-amino)-3-phenyl-1H-indole-2-carboxylic acid (4-carbamoyl-phenyl)-amide
5-(4-tert-Butyl-benzenesulfonyl-amino)-3-phenyl-1H-indole-2-carboxylic acid (3-methyl-pyridin-2-yl)-amide
5-(4-tert-Butyl-benzenesulfonyl-amino)-3-phenyl-1H-indole-2-carboxylic acid (2-chloro-phenyl)-amide
5-(4-tert-Butyl-benzenesulfonyl-amino)-3-phenyl-1H-indole-2-carboxylic acid (5-methyl-2H-pyrazol-3-yl)-amide
5-(4-tert-Butyl-benzenesulfonyl-amino)-3-phenyl-1H-indole-2-carboxylic acid quinolin-5-ylamide
5-(4-tert-Butyl-benzenesulfonyl-amino)-3-phenyl-1H-indole-2-carboxylic acid quinolin-6-ylamide
5-(4-tert-Butyl-benzenesulfonyl-amino)-3-phenyl-1H-indole-2-carboxylic acid (2,6-dichloro-pyridin-4-yl)-amide
5-(4-tert-Butyl-benzenesulfonyl-amino)-3-phenyl-1H-indole-2-carboxylic acid (3-fluoro-phenyl)-amide
5-(4-tert-Butyl-benzenesulfonyl-amino)-3-phenyl-1H-indole-2-carboxylic acid (2-methyl-pyridin-4-yl)-amide
5-(4-tert-Butyl-benzenesulfonyl-amino)-3-phenyl-1H-indole-2-carboxylic acid (3-fluoro-pyridin-4-yl)-amide
5-(4-tert-Butyl-benzenesulfonyl-amino)-3-phenyl-1H-indole-2-carboxylic acid (3-methyl-pyridin-4-yl)-amide′
5-(4-tert-Butyl-benzenesulfonyl-amino)-3-phenyl-1H-indole-2-carboxylic acid (3-bromo-pyridin-4-yl)-amide
5-(4-tert-Butyl-benzenesulfonyl-amino)-3-phenyl-1H-indole-2-carboxylic acid (2,3-dihydroxy-propyl)-amide
5-(4-tert-Butyl-benzenesulfonyl-amino)-3-phenyl-1H-indole-2-carboxylic acid (2-oxo-tetrahydro-thiophen-3-yl)-amide
5-(4-tert-Butyl-benzenesulfonyl-amino)-3-phenyl-1H-indole-2-carboxylic acid [2-(2-oxo-imidazolidin-1-yl)-ethyl]-amide
5-(4-tert-Butyl-benzenesulfonyl-amino)-3-phenyl-1H-indole-2-carboxylic acid (2,2-diethoxy-ethyl)-amide
5-(4-tert-Butyl-benzenesulfonyl-amino)-3-phenyl-1H-indole-2-carboxylic acid (3-ethoxy-propyl)-amide
5-(4-tert-Butyl-benzenesulfonyl-amino)-3-phenyl-1H-indole-2-carboxylic acid (3-isopropoxy-propyl)-amide
5-(4-tert-Butyl-benzenesulfonyl-amino)-3-phenyl-1H-indole-2-carboxylic acid (3-morpholin-4-yl-propyl)-amide
5-(4-tert-Butyl-benzenesulfonyl-amino)-3-phenyl-1H-indole-2-carboxylic acid (3-diethylamino-propyl)-amide
5-(4-tert-Butyl-benzenesulfonyl-amino)-3-phenyl-1H-indole-2-carboxylic acid (3-dimethylamino-propyl)-amide
5-(4-tert-Butyl-benzenesulfonyl-amino)-3-phenyl-1H-indole-2-carboxylic acid (furan-2-ylmethyl)amide
5-(4-tert-Butyl-benzenesulfonyl-amino)-3-phenyl-1H-indole-2-carboxylic acid (2-methylsulfanyl-ethyl)-amide
5-(4-tert-Butyl-benzenesulfonyl-amino)-3-phenyl-1H-indole-2-carboxylic acid (2-diethylamino-ethyl)-amide
5-(4-tert-Butyl-benzenesulfonyl-amino)-3-phenyl-1H-indole-2-carboxylic acid [2-(3,4-dimethoxy-phenyl)-ethyl]-amide
5-(4-tert-Butyl-benzenesulfonyl-amino)-3-phenyl-1H-indole-2-carboxylic acid (2-piperidin-1-yl-ethyl)-amide
5-(4-tert-Butyl-benzenesulfonyl-amino)-3-phenyl-1H-indole-2-carboxylic acid (3-pyrrolidin-1-yl-propyl)-amide
5-(4-tert-Butyl-benzenesulfonyl-amino)-3-phenyl-1H-indole-2-carboxylic acid phenethyl-amide
5-(4-tert-Butyl-benzenesulfonyl-amino)-3-phenyl-1H-indole-2-carboxylic acid (2-methoxy-1-methyl-ethyl)-amide
5-(4-tert-Butyl-benzenesulfonyl-amino)-3-phenyl-1H-indole-2-carboxylic acid (pyridin-2-ylmethyl)-amide
5-(4-tert-Butyl-benzenesulfonyl-amino)-3-phenyl-1H-indole-2-carboxylic acid (pyridin-3-ylmethyl)-amide
5-(4-tert-Butyl-benzenesulfonyl-amino)-3-phenyl-1H-indole-2-carboxylic acid (pyridin-4-ylmethyl)amide
5-(4-tert-Butyl-benzenesulfonyl-amino)-3-phenyl-1H-indole-2-carboxylic acid (4-diethylamino-1-methyl-butyl)-amide
5-(4-tert-Butyl-benzenesulfonyl-amino)-3-phenyl-1H-indole-2-carboxylic acid (2-imidazol-1-yl-ethyl)-amide
5-(4-tert-Butyl-benzenesulfonyl-amino)-3-phenyl-1H-indole-2-carboxylic acid benzylamide
5-(4-tert-Butyl-benzenesulfonyl-amino)-3-phenyl-1H-indole-2-carboxylic acid (2,2,2-trifluoro-ethyl)-amide
5-(4-tert-Butyl-benzenesulfonyl-amino)-3-phenyl-1H-indole-2-carboxylic acid 4-methoxy-benzylamide
5-(4-tert-Butyl-benzenesulfonyl-amino)-3-phenyl-1H-indole-2-carboxylic acid cyclopentylamide
5-(4-tert-Butyl-benzenesulfonyl-amino)-3-phenyl-1H-indole-2-carboxylic acid (3-methyl-butyl)-amide
5-(4-tert-Butyl-benzenesulfonyl-amino)-3-phenyl-1H-indole-2-carboxylic acid [3-(4-methyl-piperazin-1-yl)-propyl]-amide
5-(4-tert-Butyl-benzenesulfonyl-amino)-3-phenyl-1H-indole-2-carboxylic acid [2-(4-hydroxy-phenyl)-ethyl]-amide
5-(4-tert-Butyl-benzenesulfonyl-amino)-3-phenyl-1H-indole-2-carboxylic acid [2-(4-chloro-phenyl)-ethyl]-amide
5-(4-tert-Butyl-benzenesulfonyl-amino)-3-phenyl-1H-indole-2-carboxylic acid cyclopropylamide
5-(4-tert-Butyl-benzenesulfonyl-amino)-3-phenyl-1H-indole-2-carboxylic acid cyclohexylmethyl-amide
5-(4-tert-Butyl-benzenesulfonyl-amino)-3-phenyl-1H-indole-2-carboxylic acid (tetrahydro-furan-2-ylmethyl)-amide
5-(4-tert-Butyl-benzenesulfonyl-amino)-3-phenyl-1H-indole-2-carboxylic acid (thiophen-2-ylmethyl)amide
5-(4-tert-Butyl-benzenesulfonyl-amino)-3-phenyl-1H-indole-2-carboxylic acid 4-fluoro-benzylamide
5-(4-tert-Butyl-benzenesulfonyl-amino)-3-phenyl-1H-indole-2-carboxylic acid (2-thiophen-2-yl-ethyl)-amide
5-(4-tert-Butyl-benzenesulfonyl-amino)-3-phenyl-1H-indole-2-carboxylic acid (2-pyrrolidin-1-yl-ethyl)-amide
5-(4-tert-Butyl-benzenesulfonyl-amino)-3-phenyl-1H-indole-2-carboxylic acid 4-methyl-benzylamide
5-(4-tert-Butyl-benzenesulfonyl-amino)-3-phenyl-1H-indole-2-carboxylic acid (1-ethyl-pyrrolidin-2-ylmethyl)-amide
5-(4-tert-Butyl-benzenesulfonyl-amino)-3-phenyl-1H-indole-2-carboxylic acid (2-pyridin-3-yl-ethyl)-amide
5-(4-tert-Butyl-benzenesulfonyl-amino)-3-phenyl-1H-indole-2-carboxylic acid 3-chloro-benzylamide
5-(4-tert-Butyl-benzenesulfonyl-amino)-3-phenyl-1H-indole-2-carboxylic acid [2-(3-chloro-phenyl)-ethyl]-amide
5-(4-tert-Butyl-benzenesulfonyl-amino)-3-phenyl-1H-indole-2-carboxylic acid ((R)-2-hydroxy-1-phenyl-ethyl)-amide
5-(4-tert-Butyl-benzenesulfonyl-amino)-3-phenyl-1H-indole-2-carboxylic acid [3-(2-methyl-piperidin-1-yl)-propyl]-amide
5-(4-tert-Butyl-benzenesulfonyl-amino)-3-phenyl-1H-indole-2-carboxylic acid (3-phenyl-propyl)-amide
5-(4-tert-Butyl-benzenesulfonyl-amino)-3-phenyl-1H-indole-2-carboxylic acid (2-carbamoyl-ethyl)-amide
5-(4-tert-Butyl-benzenesulfonyl-amino)-3-phenyl-1H-indole-2-carboxylic acid [3-(5-methyl-1H-pyrazol-4-yl)-propyl]-amide
5-(4-tert-Butyl-benzenesulfonyl.-amino)-3-phenyl-1H-indole-2-carboxylic acid (4-methyl-cyclohexyl)-amide
5-(4-tert-Butyl-benzenesulfonyl-amino)-3-phenyl-1H-indole-2-carboxylic acid ((S)-2-methoxy-1-methyl-ethyl)-amide
5-(4-tert-Butyl-benzenesulfonyl-amino)-3-phenyl-1H-indole-2-carboxylic acid cyclopropylmethyl-amide
5-(4-tert-Butyl-benzenesulfonyl.-amino)-3-phenyl-1H-indole-2-carboxylic acid carbamoylmethyl-amide
5-(4-tert-Butyl-benzenesulfonyl-amino)-3-phenyl-1H-indole-2-carboxylic acid cycloheptylamide
5-(4-tert-Butyl-benzenesulfonyl-amino)-3-phenyl-1H-indole-2-carboxylic acid ((S)-2-methoxy-1-methyl-ethyl)-amide
5-(4-tert-Butyl-benzenesulfonyl-amino)-3-phenyl-1H-indole-2-carboxylic acid (furan-3-ylmethyl)-amide
5-(4-tert-Butyl-benzenesulfonyl-amino)-3-phenyl-1H-indole-2-carboxylic acid 3-fluoro-benzylamide
5-(4-tert-Butyl-benzenesulfonyl-amino)-3-phenyl-1H-indole-2-carboxylic acid (5-methyl-pyrazin-2-ylmethyl)amide
5-(4-tert-Butyl-benzenesulfonyl-amino)-3-phenyl-1H-indole-2-carboxylic acid (2-pyridin-2-yl-ethyl)-amide
5-(4-tert-Butyl-benzenesulfonyl-amino)-3-phenyl-1H-indole-2-carboxylic acid (2-phenoxy-ethyl)-amide
5-(4-tert-Butyl-benzenesulfonyl-amino)-3-phenyl-1H-indole-2-carboxylic acid (2-benzoimidazol-1-yl-ethyl)-amide
5-(4-tert-Butyl-benzenesulfonyl-amino)-3-phenyl-1H-indole-2-carboxylic acid (3-imidazol-1-yl-propyl)-amide
5-(4-tert-Butyl-benzenesulfonyl-amino)-3-phenyl-1H-indole-2-carboxylic acid (1-benzyl-piperidin-4-yl)-amide
5-(4-tert-Butyl-benzenesulfonyl-amino)-3-phenyl-1H-indole-2-carboxylic acid [3-(2-oxo-pyrrolidin-1-yl)-propyl]-amide
5-(4-tert-Butyl-benzenesulfonyl-amino)-3-phenyl-1H-indole-2-carboxylic acid [2-(1-methyl-pyrrolidin-2-yl)-ethyl]-amide
5-(4-tert-Butyl-benzenesulfonyl-amino)-3-phenyl-1H-indole-2-carboxylic acid methyl-(2-morpholin-4-yl-ethyl)-amide

The compounds according to the invention inhibit the soluble adenylate cyclase, upon which their action is based, for example, in the case of male birth control.

Adenylate cyclases are the effector molecules for one of the most used signal transduction methods; they synthesize the second messenger molecule of cyclic adenosine monophospate (CAMP) from adenosine triphosphate (ATP) with cleavage of pyrophosphate (PP). cAMP mediates numerous cellular responses for a number of neurotransmitters and hormones. The soluble, sperm-specific adenylate cyclase (sAC, human mRNA sequence (gene bank) NM_{—}018417, human gene ADCY X) is one of ten described adenylate cyclases in the human genome. In this case, sAC shows several specific properties that are distinguished from other adenylate cyclases. In contrast to all other adenylate cyclases, sAC is stimulated by the concentration of bicarbonate in the medium that surrounds it and not by G-proteins. sAC does not have any transmembrane regions in its amino acid sequence; it cannot be inhibited by forskolin, can be stimulated much more strongly by manganese than by magnesium, and shows only low sequence homologies to the other adenylate cyclases (≦26% identity of the catalytic domains I and II of sAC with other adenylate cyclases on the amino acid plane).

Specific, manganese-dependent activity of sAC was first described by T. Braun et al. (1975, PNAS 73:1097 ff) in rat testes and sperm. N. Okamura et al. (1985, J. Biol. Chem 260(17): 9699 ff) showed that the substance that stimulates the activity of sAC in the pig seminal fluid is bicarbonate. It could also be shown that only in the rat testis and sperm, but not in another tissues, AC activity that can be stimulated by bicarbonate can be detected. sAC was purified from the rat testis by the Buck and Levin group and sequenced for the first time (J. Buck et al. 1999 PNAS 96:79 ff, WO 01/85753). The properties that are to be expected (e.g., the ability to stimulate bicarbonate and magnesium) were confirmed in recombinantly-expressed proteins (Y. Chen et al. 2000 Science 289:625 ff).

Data regarding the distribution of sAC mRNA and the sAC activity that can be stimulated by bicarbonate can indicate a testis- and sperm-specific expression of the enzyme (M. L. Sinclair et al. 2000 Mol Reprod Develop 56:6 ff; N. Okamura et al. 1985, J. Biol. Chem 260(17):9699 ff; J. Buck et al. 1999 PNAS 96:79 ff). In this case, in the testicles, sAC mRNA is expressed only in later stages, the gametes that develop into sperm, but not in somatic cells (M. L. Sinclair et al. 2000 Mol Reprod Develop 56:6 ff).

Regarding the function of sAC in sperm in mammals, there are a number of pharmacological studies. Before sperm penetrate the zona pellucida of the egg, so as to subsequently merge with the oolemma of the egg, they must be prepared for this functionality. This process, the sperm capacitation, is quite well studied. A capacitated sperm is distinguished by an altered pattern of movement and by the ability to go through the process of acrosomal reaction by a suitable stimulus (a release of lytic enzymes that are presumably used in the penetration of the zona pellucida by the sperm). The sperm capacitation is carried out in vivo and in vitro, i.a., based on an elevated bicarbonate concentration in the medium (P. E. Visconti & G. S. Kopf (1998) Biol Reprod 59:1ff; E. de Lamirande et al. 1997 Mol Hum Reprod 3(3):175 ff). The sperm capacitation can also be stimulated by the addition of suitable membrane-penetrating cAMP analogs, e.g., db-cAMP and an inhibitor that inhibits their degradation (e.g., IBMX). The expected dependence of the sperm function of sAC was confirmed only recently by a genetic deletion model, a so-called knock-out mouse (G. Esposito et al. 2004 PNAS101(9):2993 ff). Male mice in which the gene for sAC is lacking show a normal spermatogenesis but are infertile. The sperm have motility defects and are not able to fertilize an egg. The animals show no other defects or abnormal findings, which corresponds to other hypothesized functions of the sAC (J. H. Zippin et al 2003 FASEB 17:82 ff)).

The sAC has a unique sequence and only a slight homology to other somatic adenylate cyclases. It is the sole adenylate cyclase in mammal sperm, and the activity is essential to the mobility of the sperm and the capacitation. Specific inhibitors of sAC accordingly represent an important possibility of regulating male fertility.

Pharmaceutical agents that contain at least one of the compounds according to claims 1-3 are therefore subjects of this invention.

The use of the compounds according to claims 1-3 is also a subject of this invention.

To use the compounds according to the invention as pharmaceutical agents, the latter are brought into the form of a pharmaceutical preparation that in addition to the active ingredient for enteral or parenteral administration contains suitable pharmaceutical, organic or inorganic inert carrier materials, such as, for example, water, gelatin, gum arabic, lactose, starch, magnesium stearate, talc, vegetable oils, polylalkylene glycols, etc. The pharmaceutical preparations can be present in solid form, for example as tablets, coated tablets, suppositories, capsules, or in liquid form, for example as solutions, suspensions, or emulsions. Moreover, they optionally contain adjuvants, such as preservatives, stabilizers, wetting agents or emulsifiers, salts for altering the osmotic pressure, or buffers. These pharmaceutical preparations are also subjects of this invention.

For parenteral administration, in particular injection solutions or suspensions, in particular aqueous solutions of active compounds in polyhydroxyethoxylated castor oil are suitable.

As carrier systems, surface-active adjuvants, such as salts of bile acids or animal or plant phospholipids, but also mixtures thereof as well as liposomes or their components can also be used.

For oral administration, in particular tablets, coated tablets or capsules with talc and/or hydrocarbon vehicles or binders, such as, for example, lactose, corn or potato starch, are suitable. The administration can also be carried out in liquid form, such as for example as a juice, to which optionally a sweetener is added.

Suppositories for example are suitable and customary for vaginal administration

The enteral, parenteral, vaginal and oral administrations are also subjects of this invention.

The dosage of the active ingredients can vary depending on the method of administration, age and weight of the patient, type and severity of the disease to be treated and similar factors. The daily dose is 0.5-1000 mg, preferably 50-200 mg, whereby the dose can be given as a single dose that is to be administered once or subdivided into 2 or more daily doses.

The compounds of general formula I according to the invention are, i.a., excellent inhibitors of the soluble adenylate cyclase. Inhibitors of the soluble adenylate cyclase lead to a reduction of the cAMP signal. The cAMP level is decisive for the monitoring of the processes that play an important role in cell proliferation, cell differentiation and apoptosis. Diseases, such as, e.g., cancer, in which the reduction of the cAMP level is decisive, can be modulated by inhibitors of soluble adenylate cyclase. This modulation can have prophylactic and therapeutic effects for the patients that suffer from such a disease. Diseases that, like cancer, are accompanied by an elevated cell proliferation are currently treated by, e.g., radiation therapy and chemotherapy. These processes are unspecific and have a high potential for side effects. The preparation of new substances that directly attack specific target sites is therefore advantageous. Substances that modulate the cAMP production by the inhibition of soluble adenylate cyclases are subjects of this invention. Thus, for example, the anomal cell proliferation can be reduced or inhibited by regulation or inhibition of the cAMP production. By the use of the substances according to the invention, the soluble adenylate cyclase can be inhibited; this has the result of a reduction of the cell proliferation. Subjects of this invention are pharmaceutical agents for treating diseases that contain at least one compound according to general formula I, as well as pharmaceutical agents with suitable formulation substances and vehicles. The diseases are thus characterized in that they are caused by disorders of the metabolism of the second messenger cAMP.

A reduction of the cAMP concentration by inhibition of the soluble adenylate cyclase can make available agents for modulation of the sperm capacitation. A subject of this invention is the use of the substances according to the invention for reduction and/or inhibition of male gamete fertility mediated by the reduction or inhibition of soluble adenylate cyclase activity and the thus resulting sperm capacitation.

The fertilization of the ovum can be prevented by the administration of an effective amount of a substance that results in the inhibition of the cAMP production. The use of the compound of general formula I for the production of a pharmaceutical agent for non-hormonal contraception is also a subject of this invention.

If the production of the starting compounds is not described, the latter are known or can be produced analogously to known compounds or to processes that are described here. It is also possible to implement all reactions that are described here in parallel reactors or by means of combinatory operating procedures.

The isomer mixtures can be separated into enantiomers or E/Z isomers according to commonly used methods, such as, for example, crystallization, chromatography or salt formation.

The production of salts is carried out in the usual way by a solution of the compound of formula I being mixed with the equivalent amount of or an excess of a base or acid, which optionally is in solution, and the precipitate being separated or the solution being worked up in the usual way.

### PRODUCTION OF THE COMPOUNDS ACCORDING TO THE INVENTION

The examples below explain the production of the compounds of general formula (I) according to the invention, without limiting the scope of the claimed compounds to these examples.

The compounds of general formula (I) according to the invention can be produced as described below.

Instructions 1: Amide Coupling:

A carboxylic acid (1.0 equivalent) is dissolved in N,N-dimethylformamide (DMF) (10 ml/1 mmol), mixed with N-[(dimethylamino)-1H-1,2,3-triazolo[4,5-b]pyridin-1-ylmethylene]-N-methylmethanaminiumhexafluorophosphate-N-oxide (HATU) (1.1 equivalent) and the amine to be coupled (1.0 equivalent). Then, ethyldiisopropylamine (1.1 equivalent) is added thereto at 0° C., and the mixture is stirred for 22 hours at room temperature. Then, the mixture is mixed with ice water (35 ml/1 mmol of carboxylic acid) and stirred for 30 minutes at room temperature. The precipitated crystals are suctioned off and dried in air. The product is either reacted without additional purification or is purified by chromatography in the next step.

Instructions 2: Reduction of the Nitro Group:

The nitro compound (1.0 equivalent) is introduced into methanol (10 ml/1 mmol) and water (0.03 ml/1 mmol), mixed with ammonium formate (5 equivalents) and with catalytic amounts of palladium on carbon (10%) and refluxed for 3 hours at 90° C. Then, it is suctioned off over Celite and rewashed with boiling methanol. After the solvent is removed, the residue is mixed with water (7 ml/1 mmol of amide), and the precipitated crystals are suctioned off. If no crystals are formed, the aqueous phase is extracted with ethyl acetate or dichloromethane. The combined organic phases are washed with saturated sodium chloride solution and dried on sodium sulfate. Then, the solvent is removed under reduced pressure.

Instructions 3: Coupling with Arylsulfonyl Chlorides:

The amine that is produced (1.0 equivalent) is dissolved in DMF (10 ml/1 mmol), mixed at 0° C. with ethyldiisopropylamine (1.5 equivalents) and arylsulfonic acid chloride (1.0 equivalent) and stirred for one hour at room temperature. The solvent is removed under reduced pressure, and the residue is purified by chromatography.

Instructions 4: Bromination:

5-Nitroindole-2-carboxylic acid ethyl ester (1.0 equivalent) is dissolved in tetrahydrofuran (5 ml/1 mmol) and mixed with N-bromosuccinimide (1.0 equivalent). After 30 minutes, water is added, and 20 minutes later, the precipitated crystals are suctioned off. If no crystals form, the aqueous phase is extracted with ethyl acetate, and the combined organic phases are dried on sodium sulfate. After the solvent is removed, the chromatographic purification of the residue takes place.

Instructions 5 Saponification:

The ester compounds (1.0 equivalent) are mixed with 19 equivalents of a 1 M sodium hydroxide solution in ethanol/water (1/1). After 6 hours at room temperature, ethanol is removed under reduced pressure, diluted with water, and a pH of 2 is set with 10% aqueous sulfuric acid. Then, the precipitated crystals are suctioned off.

Instructions 6 Coupling with Arylboronic Acids:

3-Bromoindole-2-carboxylic acid ester (1.0 equivalent) is suspended with an arylboronic acid (1.5 equivalents) in toluene/ethanol 1:1 (40 ml/1 mmol of ester) and mixed with 1 M sodium carbonate solution (2.5 equivalents) as well as lithium chloride (2.8 equivalents). After tetrakis(triphenylphosphine)-palladium (0.08 equivalent) is added, the reaction mixture is refluxed for 8 hours. After being cooled to room temperature, it is diluted with ethyl acetate (70 ml/1 mmol of ester) and suctioned off over Celite 10 minutes later. The filtrate is washed with saturated sodium bicarbonate solution and saturated sodium chloride solution and dried on sodium sulfate. After the solvent is removed, the chromatographic purification of the residue is carried out.

### Example 1

### 5-(4-tert-Butylbenzenesulfonylamino)-3-phenyl-1H-indole-2-carboxylic acid-cyclopropylamide

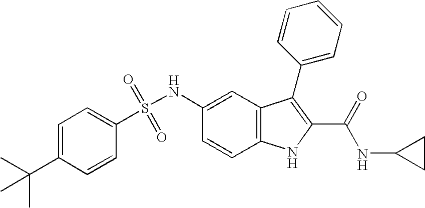

According to Instructions 1, after the reaction of 5-nitro-3-phenyl-1H-indole-2-carboxylic acid (423 mg, 1.5 mmol) with cyclopropylamine (0.105 ml, 1.5 mmol), 445 mg (93%) of the product 5-nitro-3-phenyl-1H-indole-2-carboxylic acid cyclo-propylamide, which is reacted without additional purification in the step below, is obtained.

According to Instructions 2, after the reaction of 5-nitro-3-phenyl-1H-indole-2-carboxylic acid cyclopropylamide (445 mg, 1.39 mmol) with ammonium formate (438 mg, 6.95 mmol) in the presence of palladium on carbon (44 mg), 289 mg (72%) of the product 5-amino-3-phenyl-1H-indole-2-carboxylic acid cyclopropylamide is obtained.

NMR (300 MHz, DMSO-d6): δ 0.28-0.36 (m, 2H), 0.60-0.68 (m, 2H), 2.72 (m, 1H), 5.00 (br, 2H), 6.60-6.68 (m, 2H), 7.15 (d, 1H), 7.22-7.38 (m, 2H), 7.40-7.50 (m, 4H), 11.19 (s, 11H).

According to Instructions 3, after the reaction of 5-amino-3-phenyl-1H-indole-2-carboxylic acid cyclopropylamide (145 mg, 0.5 mmol) with 4-tert-butylbenzenesulfonic acid chloride (116 mg, 0.5 mmol) and chromatographic purification (silica gel, hexane/ethyl acetate (0-100% ethyl acetate)), 190 mg (78%) of the product 5-(4-tert-butylbenzenesulfonylamino)-3-phenyl-1H-indole-2-carboxylic acid-cyclopropylamide is obtained.

NMR (300 MHz, DMSO-d6): δ 0.32-0.37 (m, 2H), 0.59-0.65 (m, 2H), 1.25 (s, 9H), 2.67-2.76 (m, 1H), 7.02 (dd, 1H), 7.10 (s, 1H), 7.25 (d, 2H), 7.31-7.36 (m, 2H), 7.40-7.45 (m, 2H), 7.51-7.59 (m, 5H), 9.79 (s, 1H), 11.66 (s, 1H).

### Example 2

### 5-(4-tert-Butylbenzenesulfonylamino)-3-phenyl-1H-indole-2-carboxylic acid-pyridin-3-ylamide

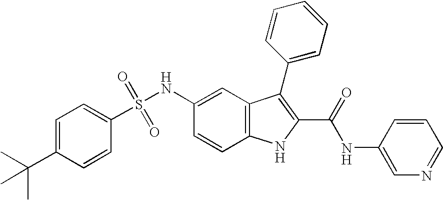

According to Instructions 1, after the reaction of 5-nitro-3-phenyl-1H-indole-2-carboxylic acid (423 mg, 1.5 mmol) with 3-aminopyridine (141 mg, 1.5 mmol), 494 mg (93%) of the product 5-nitro-3-phenyl-1H-indole-2-carboxylic acid pyridin-3-ylamide, which is reacted without additional purification in the step below, is obtained

According to Instructions 2, after the reaction of 5-nitro-3-phenyl-1H-indole-2-carboxylic acid pyridin-3-ylamide (494 mg, 1.39 mmol) with ammonium formate (435 mg, 6.90 mmol) in the presence of palladium on carbon (49 mg), 257 mg (72%) of the product 5-amino-3-phenyl-1H-indole-2-carboxylic acid pyridin-3-ylamide is obtained.

NMR (300 MHz, DMSO-d6): δ 4.98 (br, 2H), 6.70-6.78 (m, 2H), 7.23 (d, 1H), 7.30-7.38 (m, 2H), 7.40-7.58 (m, 4H), 8.00 (d, 1H), 8.25 (dd, 1H), 8.64 (d, 1H), 9.75 (s, 1H), 11.49 (s, 1H).

According to Instructions 3, after the reaction of (128 mg, 0.39 mmol) with 4-tert-butylbenzenesulfonic acid chloride (90 mg, 0.39 mmol) and chromatographic purification (silica gel, hexane/ethyl acetate (0-100% ethyl acetate)), 140 mg (68%) of the product 5-(4-tert-butylbenzenesulfonylamino)-3-phenyl-1H-indole-2-carboxylic acid-pyridin-3-ylamide is obtained.

NMR (300 MHz, DMSO-d6): δ 1.26 (s, 9H), 7.09 (dd, 1H), 7.19 (s, 1H), 7.34-7.43 (m, 7H), 7.54 (d, 2H), 7.60 (d, 2H), 7.95-7.97 (m, 1H), 8.27 (d, 1H), 8.65 (s, 1H), 9.86 (s, 1H), 9.98 (s, 1H), 11.97 (s, 1H).

### Example 3

### 5-(4-tert-Butylbenzenesulfonylamino)-3-phenyl-1H-indole-2-carboxylic acid-cyclohexylamide

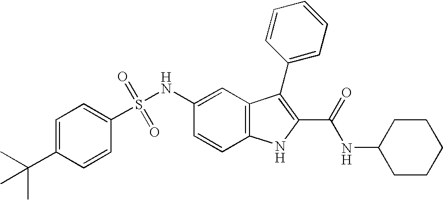

According to Instructions 1, after the reaction of 5-nitro-3-phenyl-1H-indole-2-carboxylic acid (423 mg, 1.5 mmol) with cyclohexylamine (149 mg, 1.5 mmol), 504 mg (93%) of the product 5-nitro-3-phenyl-1H-indole-2-carboxylic acid cyclohexyl amide, which is used without additional purification in the step below, is obtained.

According to Instructions 2, after the reaction of 5-nitro-3-phenyl-1H-indole-2-carboxylic acid cyclohexyl amide (504 mg, 1.39 mmol) with ammonium formate (438 mg, 6.95 mmol) in the presence of palladium on carbon (50 mg), 367 mg (79%) of the product 5-amino-3-phenyl-1H-indole-2-carboxylic acid cyclohexyl amide is obtained.

NMR (300 MHz, DMSO-d6): δ 0.92-1.16 (m, 3H), 1.18-1.31 (m, 2H), 1.38-1.55 (m, 3H), 1.62-1.76 (m, 2H), 3.61-3.78 (m, 1H), 4.82 (br, 2H), 6.58-6.74 (m, 3H), 7.18 (d, 1H), 7.32-7.40 (m, 1H), 7.43-7.54 (m, 4H), 11.25 (s, 1H).

According to Instructions 3, after the reaction of 5-amino-3-phenyl-1H-indole-2-carboxylic acid cyclohexylamide (183 mg, 0.55 mmol) with 4-tert-butylbenzenesulfonic acid chloride (128 mg, 0.55 mmol) and chromatographic purification (silica gel, hexane/ethyl acetate (0-100% ethyl acetate)), 231 mg (79%) of the product 5-(4-tert-butylbenzenesulfonylamino)-3-phenyl-1H-indole-2-carboxylic acid-cyclohexylamide is obtained.

NMR (300 MHz, DMSO-d6): δ 0.98-1.09 (m, 3H), 1.19-1.32 (m, 11H), 1.50-1.53 (m, 3H), 1.66-1.70 (m, 2H), 3.65-3.74 (m, 1H), 6.97-7.06 (m, 3H), 7.29-7.48 (m, 6H), 7.51 (d, 2H), 7.57 (d, 2H), 9.79 (s, 1H), 11.71 (s, 1H).

### Example 4

### 4-tert-Butyl-N-[3-phenyl-2-(pyrrolidine-1-carbonyl)-1H-indol-5-yl]-benzenesulfonamide

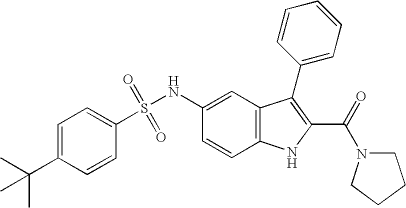

According to Instructions 1, after the reaction of 5-nitro-3-phenyl-1H-indole-2-carboxylic acid (423 mg, 1.5 mmol) with pyrrolidine (0.124 mg, 1.5 mmol), 430 mg (85%) of the product 5-nitro-3-phenyl-1H-indol-2-yl)pyrrolidin-1-yl-methanone, which is reacted without additional purification in the step below, is obtained.

According to Instructions 2, after the reaction of 5-nitro-3-phenyl-1H-indol-2-yl)pyrrolidin-1-yl-methanone (430 mg, 1.28 mmol) with ammonium formate (404 mg, 6.41 mmol) in the presence of palladium on carbon (43 mg), 255 mg (65%) of the product (5-amino-3-phenyl-1H-indol-2-yl)pyrrolidin-1-yl-methanone is obtained.

NMR (300 MHz, DMSO-d6): δ 1.44-1.60 (m, 2H), 1.62-1.78 (m, 2H), 2.84 (t, 2H), 3.41 (t, 2H), 4.90 (br, 2H), 6.61 (dd, 1H), 6.88 (d, 1H), 7.12 (d, 1H), 7.22-7.30 (m, 1H), 7.38-7.50 (m, 4H), 11.25 (s, 1H).

According to Instructions 3, after the reaction of (5-amino-3-phenyl-1H-indol-2-yl)pyrrolidin-1-yl-methanone (128 mg, 0.42 mmol) with 4-tert-butylbenzenesulfonic acid chloride (98 mg, 0.42 mmol) and chromatographic purification (silica gel, hexane/ethyl acetate (0-100% ethyl acetate)), 160 mg (76%) of the product 4-tert-butyl-N-[3-phenyl-2-(pyrrolidine-1-carbonyl)-1H-indol-5-yl]-benzenesulfonamide is obtained.

NMR (300 MHz, DMSO-d6): δ 1.24 (s, 9H), 1.49-1.55 (m, 2H), 1.65-1.73 (m, 2H), 2.81 (t, 2H), 3.41 (t, 2H), 7.01 (dd, 1H), 7.24-7.34 (m, 5H), 7.42-7.46 (m, 2H), 7.54 (d, 2H), 7.60 (d, 2H), 9.85 (s, 1H), 11.72 (s, 1H).

### Example 5

### 4-tert-Butyl-N-[2-(morpholine-4-carbonyl)-3-phenyl-1H-indol-5 yl]-benzene-sulfonamide

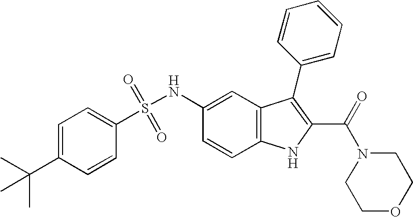

According to Instructions 1, after the reaction of 5-nitro-3-phenyl-1H-indole-2-carboxylic acid (423 mg, 1.5 mmol) with morpholine (0.131 ml, 1.5 mmol), 487 mg (92%) of the product morpholin-4-yl-(5-nitro-3-phenyl-1H-indol-2-yl)-methanone, which is reacted without additional purification in the step below, is obtained.

According to Instructions 2, after the reaction of morpholin-4-yl-(5-nitro-3-phenyl-1H-indol-2-yl)-methanone (487 mg, 1.39 mmol) with ammonium formate (438 mg, 6.95 mmol) in the presence of palladium on carbon (49 mg), 393 mg (88%) of the product (5-amino-3-phenyl-1H-indol-2yl)-morpholin-4-ylmethanone is obtained.

NMR (300 MHz, DMSO-d6): δ 2.78-3.70 (m, 8H), 4.70 (br, 2H), 6.63 (dd, 1H), 6.82 (d, 1H), 7.12 (d, 1H), 7.29-7.51 (m, 5H), 11.30 (s, 1H).

According to Instructions 3, after the reaction of (5-amino-3-phenyl-1H-indol-2yl)-morpholin-4-ylmethanone (196 mg, 0.61 mmol) with 4-tert-butylbenzenesulfonic acid chloride (142 mg, 0.61 mmol) and chromatographic purification (silica gel, hexane/ethyl acetate (0-100% ethyl acetate)), 280 mg (88%) of the product 4-tert-butyl-N-[2-(morpholine-4-carbonyl)-3-phenyl-1H-indol-5yl]-benzenesulfonamide is obtained.

NMR (300 MHz, DMSO-d6): δ 1.24 (s, 9H), 2.75-3.10 (br, 4H), 3.30-3.60 (br, 4H), 7.02 (dd, 1H), 7.21-7.30 (m, 5H), 7.42-7.61 (m, 6H), 9.85 (s, 1H), 11.78 (s, 1H).

### Example 6

### 5-(4-tert-Butylbenzenesulfonylamino)-3-phenyl-1H-indole-2-carboxylic acid-(2-dimethylaminoethyl)amide

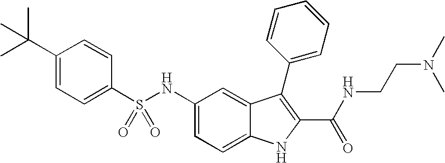

According to Instructions 1, after the reaction of 5-nitro-3-phenyl-1H-indole-2-carboxylic acid (1.0 g, 3.54 mmol) with N,N-dimethylethylenediamine (312 mg, 3.54 mmol), 557 mg (44%) of the product 5-nitro-3-phenyl-1H-indole-2-carboxylic acid-(2-dimethylaminoethyl)amide is obtained, which is reacted without additional purification in the step below.

According to Instructions 2, after the reaction of 5-nitro-3-phenyl-1H-indole-2-carboxylic acid-(2-dimethylaminoethyl)amide (557 mg, 1.58 mmol) with ammonium formate (498 mg, 7.90 mmol) in the presence of palladium on carbon (87 mg), 386 mg (75%) of the product 5-amino-3-phenyl-1H-indole-2-carboxylic acid-(2-dimethylaminoethyl)amide is obtained.

NMR (300 MHz, DMSO-d6): δ 2.03 (s, 6H), 2.21 (t, 2H), 3.24 (q, 2H), 4.95 (br, 2H), 6.55 (d, 1H), 6.64 (dd, 1H), 6.86 (t, 1H), 7.17 (d, 1H), 7.34-7.50 (m, 5H), 11.25 (s, 1H).

According to Instructions 3, after the reaction of 5-amino-3-phenyl-1H-indole-2-carboxylic acid-(2-dimethylaminoethyl)amide (229 mg, 0.71 mmol) with 4-tert-butylbenzenesulfonic acid chloride (165 mg, 0.71 mmol), chromatographic purification (silica gel, dichloromethane/methanol (0-10% methanol)), 300 mg of solid, which is dissolved in dichloromethane and washed with aqueous 1N KOH solution. After drying on sodium sulfate and after removal of the solvent, 90 mg (24%) of the product 5-(4-tert-butylbenzenesulfonylamino)-3-phenyl-1H-indole-2-carboxylic acid-(2-dimethylamino-ethyl)amide.

NMR (300 MHz, DMSO-d6): δ 1.25 (s, 9H), 2.00 (s, 6H), 2.25 (t, 2H), 3.24 (q, 2H), 7.00-7.10 (m, 3H), 7.21-7.60 (m, 10H), 9.80 (s, 1H), 11.71 (s, 1H).

### Example 7

### 5-(4-Cyanobenzenesulfonylamino)-3-phenyl-1H-indole-2-carboxylic acid propyl-amide

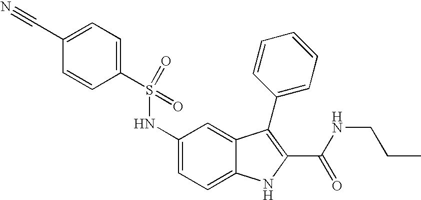

According to Instructions 1, after the reaction of 5-nitro-3-phenyl-1H-indole-2-carboxylic acid (423 mg, 1.5 mmol) with propylamine (89 mg, 1.5 mmol), 450 mg (93%) of the product 5-nitro-3-phenyl-1H-indole-2-carboxylic acid propylamide, which is reacted without additional purification in the step below, is obtained.

According to Instructions 2, after the reaction of 5-nitro-3-phenyl-1H-indole-2-carboxylic acid propylamide (400 mg, 1.24 mmol) with ammonium formate (390 mg, 6.18 mmol) in the presence of palladium on carbon (40 mg), 281 mg (77%) of the product 5-amino-3-phenyl-1H-indole-2-carboxylic acid propylamide is obtained.

NMR (300 MHz, DMSO-d6): δ 0.76 (t, 3H), 1.33-1.40 (m, 2H), 3.11 (q, 2H), 4.64 (br, 2H), 6.62-66 (m, 2H), 7.00 (t, 1H), 7.17 (d, 1H), 7.33-7.36 (m, 1H), 7.43-7.46 (m, 4H), 11.19 (s, 1H).

According to Instructions 3, after the reaction of 5-amino-3-phenyl-1H-indole-2-carboxylic acid propylamide (146 mg, 0.5 mmol) with 4-cyanobenzene sulfonyl chloride (121 mg, 0.60 mmol) and chromatographic purification (silica gel, hexane/ethyl acetate)), 41 mg (18%) of the product 5-(4-cyanobenzenesulfonylamino)-3-phenyl-1H-indole-2-carboxylic acid propylamide is obtained.

NMR (300 MHz, DMSO-d6): δ 0.76 (t, 3H), 1.31-1.43 (m, 2H), 3.11 (q, 2H), 6.96 (dd, 1H), 7.00 (s, 1H), 7.27-7.49 (m, 7H), 7.77 (d, 2H), 8.03 (d, 2H), 10.08 (s, 1H), 11.75 (s, 1H).

### Example 8

### 5-(4-Bromobenzenesulfonylamino)-3-phenyl-1H-indole-2-carboxylic acid propylamide

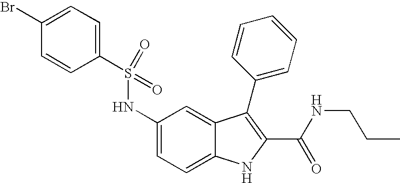

According to Instructions 3, after the reaction of 5-amino-3-phenyl-1H-indole-2-carboxylic acid propylamide (146 mg, 0.5 mmol) with 4-bromobenzenesulfonyl chloride (153 mg, 0.60 mmol) and chromatographic purification (silica gel, hexane/ethyl acetate (0-100% ethyl acetate)), 91 mg (35%) of the product is obtained.

NMR (300 MHz, DMSO-d6): δ 0.76 (t, 3H), 1.31-1.43 (m, 2H), 3.11 (q, 2H), 6.96-7.03 (m, 2H), 7.26-7.49 (m, 7H), 7.53 (d, 2H), 7.77 (d, 2H), 9.86 (s, 1H), 11.73 (s, 1H).

### Example 9

### 5-(4-tert-Butylbenzenesulfonylamino)-3-phenyl-1H-indole-2-carboxylic acid propylamide

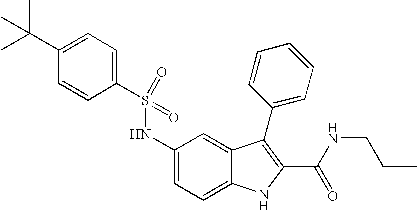

According to Instructions 3, after the reaction of 5-amino-3-phenyl-1H-indole-2-carboxylic acid propylamide (146 mg, 0.5 mmol) with 4-tert-butylbenzenesulfonic acid chloride (139 mg, 0.60 mmol) and chromatographic purification (silica gel, hexane/ethyl acetate (0-100% ethyl acetate)), 52 mg (21%) of the product is obtained.

NMR (300 MHz, CDCl₃): δ 0.73 (t, 3H), 1.29-1.41 (m, 11H), 3.22 (q, 2H), 5.92 (t, 1H), 6.47 (s, 1H), 7.04 (s, 1H), 7.07 (dd, 1H), 7.34-7.61 (m, 10H), 9.55 (s, 1H).

### Example 10

### 5-(4-(Trifluoromethoxy)benzenesulfonylamino)-3-phenyl-1H-indole-2-carboxylic acid propylamide

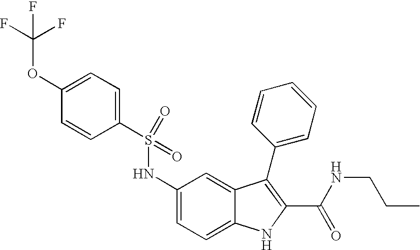

According to Instructions 3, after the reaction of 5-amino-3-phenyl-1H-indole-2-carboxylic acid propylamide (146 mg, 0.5 mmol) with 4-(trifluoromethoxy)benzene-sulfonyl chloride (156 mg, 0.60 mmol) and chromatographic purification (silica gel, hexane/ethyl acetate (0-100% ethyl acetate)), 106 mg (41%) of the product is obtained.

NMR (300 MHz, CDCl₃): δ 0.73 (t, 3H), 1.28-1.38 (m, 2H), 3.22 (q, 2H), 5.95 (t, 1H), 6.76 (s, 1H), 7.02 (s, 1H), 7.06 (dd, 1H), 7.20 (d, 2H), 7.37-7.51 (m, 6H), 7.7-1 (d, 2H), 9.81 (s, 1H).

### Example 11

### 5-(4-(Methylsulfonyl)benzenesulfonylamino)-3-phenyl-1H-indole-2-carboxylic acid propylamide

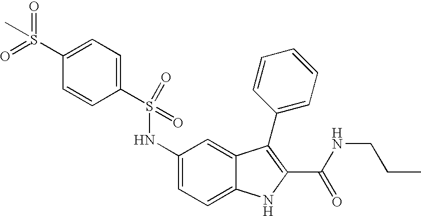

According to Instructions 3, after the reaction of 5-amino-3-phenyl-1H-indole-2-carboxylic acid propylamide (146 mg, 0.50 mmol) with 4-methylsulfonylbenzene-sulfonyl chloride (153 mg, 0.60 mmol) and chromatographic purification (silica gel, hexane/ethyl acetate (0-100% ethyl acetate)), 87 mg (39%) of the product is obtained.

NMR (300 MHz, DMSO-d6): δ 0.76 (t, 3H), 1.31-1.43 (m, 2H), 3.11 (q, 2H), 3.25 (s, 3H), 7.00 (dd, 1H), 7.05 (s, 1H), 7.27 (d, 2H), 7.34-7.38 (m, 3H), 7.43-7.49 (m, 2H), 7.87 (d, 2H), 8.10 (d, 2H), 10.10 (s, 1H), 11.75 (s, 1H).

### Example 12

### 5-(4-tert-Butylbenzenesulfonylamino)-3-phenyl-1H-indole-2-carboxylic acid phenylamide

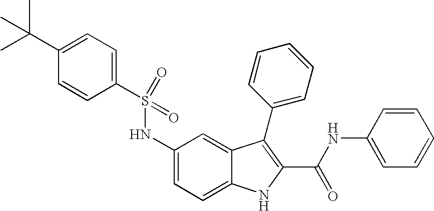

According to Instructions 1, after the reaction of 5-nitro-3-phenyl-1H-indole-2-carboxylic acid (1.27 g mg, 4.5 mmol) with aniline (419 mg, 1.5 mmol), the product 5-nitro-3-phenyl-1H-indole-2-carboxylic acid phenylamide, which is reacted without additional purification in the step below, is obtained in a quantitative yield.

According to Instructions 2, after the reaction of 5-nitro-3-phenyl-1H-indole-2-carboxylic acid phenylamide (1.61 g, 4.5 mmol) with ammonium formate (1.42 g, 22.5 mmol) in the presence of palladium on carbon (160 mg), 1.19 mg (81%) of the product 5-amino-3-phenyl-1H-indole-2-carboxylic acid phenylamide is obtained.

NMR (300 MHz, DMSO-d6): δ 4.90 (br, 2H), 6.68-6.72 (m, 2H), 7.15 (t, 1H), 7.20-7.40 (m, 4H), 7.44-7.60 (m, 6H), 9.38 (s, 1H), 11.5 (s, 1H).

According to Instructions 3, after the reaction of 5-amino-3-phenyl-1H-indole-2-carboxylic acid phenylamide (164 mg, 0.5 mmol) with 4-tert-butylbenzenesulfonic acid chloride (116 mg, 0.50 mmol) and chromatographic purification (silica gel, hexane/ethyl acetate (0-100% ethyl acetate)), 117 mg (44%) of the product 5-(4-tert-butylbenzenesulfonylamino)-3-phenyl-1H-indole-2-carboxylic acid phenylamide is obtained.

NMR (300 MHz, DMSO-d6): δ 1.26 (s, 9H), 7.08 (m, 2H), 7.17 (d, 11H), 7.28-7.51 (m, 10H), 7.55 (d, 2H), 7.59 (d, 2H), 9.65 (s, 1H), 9.85 (s, 1H), 11.94 (s, 1H).

### Example 13

### 5-(4-Cyanobenzenesulfonylamino)-3-phenyl-1H-indole-2-carboxylic acid phenylamide

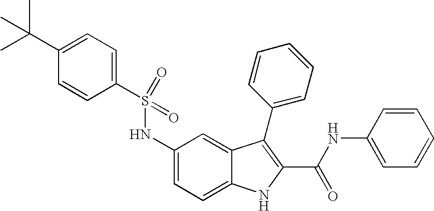

According to Instructions 3, after the reaction of 5-amino-3-phenyl-1H-indole-2-carboxylic acid phenylamide (164 mg, 0.50 mmol) with 4-cyanobenzenesulfonyl chloride (100 mg, 0.50 mmol) and chromatographic purification (silica gel, hexane/ethyl acetate (0-100% ethyl acetate)), 35 mg (14%) of the product is obtained.

NMR (300 MHz, DMSO-d6): δ 7.04-7.11 (m, 3H), 7.46-7.51 (m, 10H), 7.80 (d, 2H), 8.05 (d, 2H), 9.66 (s, 1H), 10.12 (s, 1H), 11.99 (s, 1H).

### Example 14

### 5-(4-Bromobenzenesulfonylamino)-3-phenyl-1H-indole-2-carboxylic acid phenylamide

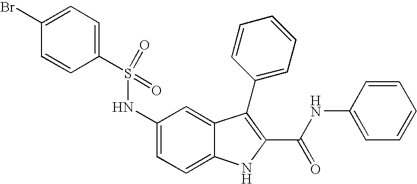

According to Instructions 3, after the reaction of 5-amino-3-phenyl-1H-indole-2-carboxylic acid phenylamide (164 mg, 0.50 mmol) with 4-bromobenzenesulfonyl chloride (127 mg, 0.50 mmol) and chromatographic purification (silica gel, hexane/ethyl acetate (0-100% ethyl acetate)), 76 mg (27%) of the product is obtained.

NMR (300 MHz, DMSO-d6): δ 7.02-7.10 (m, 3H), 7.28-7.51 (m, 10H), 7.57 (d, 2H), 7.78 (d, 2H), 9.67 (s, 1H), 9.92 (s, 1H), 11.98 (s, 1H).

### Example 15

### 5-(4-(Trifluoromethoxy)benzenesulfonylamino)-3-phenyl-1H-indole-2-carboxylic acid phenylamide

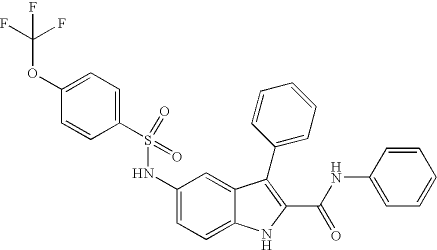

According to Instructions 3, after the reaction of 5-amino-3-phenyl-1H-indole-2-carboxylic acid phenylamide (164 mg, 0.50 mmol) with 4-(trifluoromethoxy)benzene-sulfonyl chloride (130 mg, 0.50 mmol) and chromatographic purification (silica gel, hexane/ethyl acetate (0-100% ethyl acetate)), 161 mg (58%) of the product is obtained.

NMR (300 MHz, DMSO-d6): δ 7.02-7.09 (m, 2H), 7.14 (d, 1H), 7.28-7.57 (m, 12H), 7.77 (d, 2H), 7.78 (d, 2H), 9.67 (s, 1H), 9.98 (s, 1H), 11.98 (s, 1H).

### Example 16

### 5-(4-(Methylsulfonyl)benzenesulfonylamino)-3-phenyl-1H-indole-2-carboxylic acid phenylamide

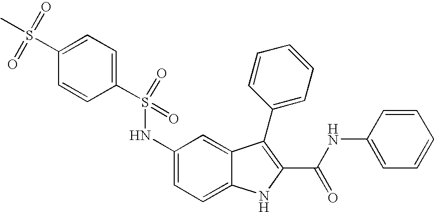

According to Instructions 3, after the reaction of 5-amino-3-phenyl-1H-indole-2-carboxylic acid phenylamide (164 mg, 0.5 mmol) with 4-methylsulfonylbenzene-sulfonyl chloride (127 mg, 0.50 mmol) and chromatographic purification (silica gel, hexane/ethyl acetate (0-100% ethyl acetate)), 140 mg (51%) of the product is obtained.

NMR (300 MHz, DMSO-d6): δ 3.27 (s, 3H), 7.03-7.09 (m, 2H), 7.16 (d, 1H), 7.28-7.51 (m, 10H), 7.91 (d, 2H), 8.12 (d, 2H), 9.66 (s, 1H), 10.15 (s, 1H), 11.99 (s, 1H).

### Example 17

### 5-(4-tert-Butylbenzenesulfonylamino)-3-phenyl-1H-indole-2-carboxylic acid-pyridin-2-ylamide

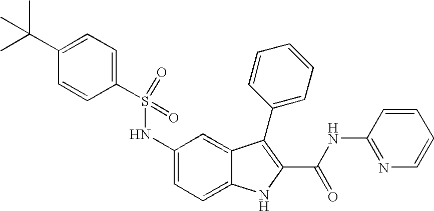

According to Instructions 1, after the reaction of 5-nitro-3-phenyl-1H-indole-2-carboxylic acid (1.27 g, 4.5 mmol) with 2-aminopyridine (423 mg, 4.5 mmol), 1.46 g (90%) of the product 5-nitro-3-phenyl-1H-indole-2-carboxylic acid-pyridin-2-ylamide, which is reacted without additional purification in the step below, is obtained.

According to Instructions 2, after the reaction of the 5-nitro-3-phenyl-1H-indole-2-carboxylic acid pyridin-2-ylamide (1.45 g, 4.05 mmol) that is obtained with ammonium formate (1.27 g, 20.23 mmol) in the presence of palladium on carbon (145 mg), 490 mg (37%) of the product 5-amino-3-phenyl-1H-indole-2-carboxylic acid pyridin-2-ylamide is obtained.

NMR (300 MHz, DMSO-d6): δ 5.40 (br, 2H), 6.57 (s, 1H), 6.73 (dd, 1H), 7.06-7.10 (m, 1H), 7.23 (d, 1H), 7.43-7.53 (m, 5H), 7.79 (td, 1H), 8.15-8.23 (m, 2H), 9.00 (s, 1H), 11.60 (s, 1H).

According to Instructions 3, after the reaction of 5-amino-3-phenyl-1H-indole-2-carboxylic acid pyridin-2-ylamide (98 mg, 0.3 mmol) with 4-tert-butylbenzenesulfonyl chloride (70 mg, 0.3 mmol) and chromatographic purification (silica gel, hexane/ethyl acetate (0-100% ethyl acetate)), 72 mg (45%) of the product 5-(4-tert-butylbenzenesulfonylamino)-3-phenyl-1H-indole-2-carboxylic acid-pyridin-2-ylamide is obtained.

NMR (300 MHz, DMSO-d6): δ 1.26 (s, 9H), 7.06-7.12 (m, 3H), 7.36-7.60 (m, 10H), 7.80 (td, 1H), 8.14 (d, 1H), 8.23 (d, 1H), 9.31 (s, 1H), 9.86 (s, 1H), 12.1 (s, 1H).

### Example 18

### 5-(4-Cyanobenzenesulfonylamino)-3-phenyl-1H-indole-2-carboxylic acid-pyridin-2-ylamide

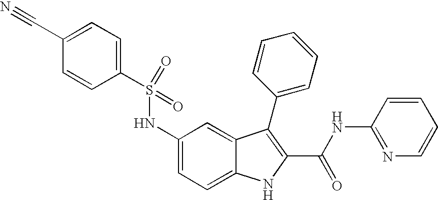

According to Instructions 3, after the reaction of 5-amino-3-phenyl-1H-indole-2-carboxylic acid pyridin-2-ylamide (98 mg, 0.3 mmol) with 4-cyanobenzenesulfonyl chloride (60 mg, 0.3 mmol) and chromatographic purification (silica gel, hexane/ethyl acetate (0-100% ethyl acetate)), 49 mg (33%) of the product is obtained.

NMR (300 MHz, DMSO-d6): δ 6.99 (s, 1H), 7.04 (dd, 1H), 7.11 (dd, 1H), 7.36-7.54 (m, 6H), 7.77-7.84 (m, 3H), 8.05 (d, 2H), 8.14 (d, 1H), 8.25 (d, 1H), 9.34 (s, 1H), 10.12 (s, 1H), 12.09 (s, 1H).

### Example 19

### 5-(4-tert-Butylbenzenesulfonylamino)-3-phenyl-1H-indole-2-carboxylic acid-(2-morpholin-4-ylethyl)amide

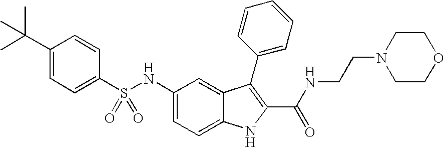

According to Instructions 1, after the reaction of 5-nitro-3-phenyl-1H-indole-2-carboxylic acid (423 mg, 1.5 mmol) with 4-(2-aminoethyl)morpholine (0.197 ml, 1.5 mmol), 528 mg (89%) of the product S-nitro-3-phenyl-1H-indole-2-carboxylic acid-(2-morpholin-4-ylethyl)amide, which is reacted without additional purification in the step below, is obtained.

According to Instructions 2, after the reaction of 5-nitro-3-phenyl-1H-indole-2-carboxylic acid-(2-morpholin-4-ylethyl)amide (521 mg, 1.32 mmol) with ammonium formate (416 mg, 6.60 mmol) in the presence of palladium on carbon (52 mg), 337 mg (70%) of the product 5-amino-3-phenyl-1H-indole-2-carboxylic acid-(2-morpholin-4-ylethyl)amide is obtained.

NMR (300 MHz, DMSO-d6): δ 2.16-2.22 (m, 4H), 2.25 (t, 2H), 3.26 (q, 2H), 3.40-3.48 (m, 4H), 4.75 (br, 2H), 6.55 (d, 1H), 6.64 (dd, 1H), 6.80 (t, 1H), 7.20 (d, 1H), 7.32-7.56 (m, 5H), 11.28 (s, 1H).

According to Instructions 3, after the reaction of 5-amino-3-phenyl-1H-indole-2-carboxylic acid-(2-morpholin-4-ylethyl)amide (330 mg, 0.91 mmol) with 4-tert-butylbenzenesulfonic acid chloride (212 mg, 0.91 mmol) and chromatographic purification (silica gel, hexane/ethyl acetate (0-100% ethyl acetate)), 130 mg (25%) of the product 5-(4-tert-butylbenzenesulfonylamino)-3-phenyl-1H-indole-2-carboxylic acid-(2-morpholin-4-ylethyl)amide is obtained.

NMR (300 MHz, DMSO-d6): δ 1.25 (s, 9H), 2.25-2.36 (m, 6H), 3.27 (q, 2H), 3.40-3.41 (m, 4H), 6.98 (t, 1H), 7.02-7.05 (m, 2H), 7.29-7.51 (m, 6H), 7.53 (d, 2H), 7.57 (d, 2H), 9.79 (s, 1H), 11.73 (s, 1H).

### Example 20

### 5-(4-tert-Butylbenzenesulfonylamino)-3-phenyl-1H-indole-2-carboxylic acid-(4-methylpiperazin-1-yl)amide

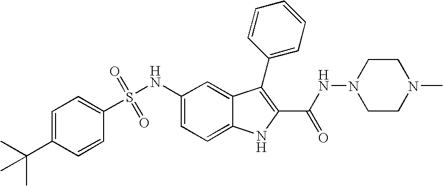

According to Instructions 1, after the reaction of 5-nitro-3-phenyl-1H-indole-2-carboxylic acid (423 mg, 1.5 mmol) with 1-amino-4-methylpiperazine (0.282 ml, 1.5 mmol), the product 5-nitro-3-phenyl-1H-indole-2-carboxylic acid-(4-methylpiperazin-1-yl)amide after chromatographic purification (silica gel, dichloromethane/methanol (0-30% methanol)) in quantitative yield.

NMR (300 MHz, DMSO-d6): δ 2.60 (s, 3H), 2.85-3.10 (m, 8H), 7.40-7.60 (m, 5H), 7.64 (d, 1H), 8.23 (dd, 1H), 8.48 (d, 1H), 9.20 (s, 1H), 12.60 (s, 1H).

According to Instructions 2, after the reaction of 5-nitro-3-phenyl-1H-indole-2-carboxylic acid-(4-methylpiperazin-1-yl)amide (830 mg, 2.19 mmol) with ammonium formate (688 mg, 10.91 mmol) in the presence of palladium on carbon (83 mg) and chromatographic purification (silica gel, dichloromethane/methanol (0-40% methanol)), 720 mg (94%) of the product 5-amino-3-phenyl-1H-indole-2-carboxylic acid-(4-methylpiperazin-1-yl)amide is obtained.

NMR (300 MHz, DMSO-d6): δ 2.35 (s, 3H), 2.60-2.80 (m, 8H), 6.60-6.70 (m, 2H), 7.14 (d, 1H), 7.25-7.35 (m, 1H), 7.38-7.50 (m, 4H), 8.42 (s, 1H), 11.25 (s, 1H).

According to Instructions 3, after the reaction of 5-amino-3-phenyl-1H-indole-2-carboxylic acid-(4-methylpiperazin-1-yl)amide (720 mg, 2.06 mmol) with 4-tert-butylbenzenesulfonic acid chloride (476 mg, 2.06 mmol) and chromatographic purification (silica gel, dichloromethane/methanol (0-30% methanol)), 170 mg (15%) of the product 5-(4-tert-butylbenzenesulfonylamino)-3-phenyl-1H-indole-2-carboxylic acid-(4-methyl-piperazin-1-yl)amide is obtained.

NMR (300 MHz, DMSO-d6): δ 1.25 (s, 9H), 2.65 (s, 3H), 3.03-3.16 (m, 8H), 7.03 (d, 1H), 7.16 (s, 1H), 7.29-7.35 (m, 4H), 7.43-7.46 (m, 2H), 7.50-7.60 (m, 4H), 9.08 (s, 1H), 9.85 (s, 1H), 11.80 (s, 1H).

### Example 21

### 5-(4-tert-Butylbenzenesulfonylamino)-3-phenyl-1H-indole-2-carboxylic acid-pyrindin-4-ylamide

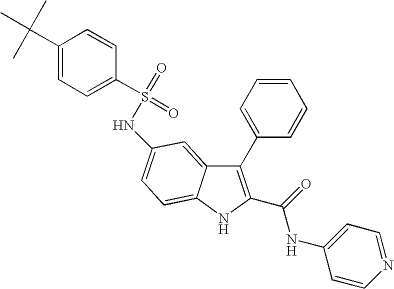

According to Instructions 1, after the reaction of 5-nitro-3-phenyl-1H-indole-2-carboxylic acid (1.0 g, 3.54 mmol) with 4-aminopyridine (382 mg, 3.54 mmol), 1.16 g (91%) of the product 5-nitro-3-phenyl-1H-indole-2-carboxylic acid pyrindin-4-ylamide, which is reacted without additional purification in the step below, is obtained.

According to Instructions 2, after the reaction of 5-nitro-3-phenyl-1H-indole-2-carboxylic acid pyrindin-4-ylamide (280 mg, 0.75 mmol) with ammonium formate (237 mg, 3.76 mmol) in the presence of palladium on carbon (28 mg), 110 mg (43%) of the product 5-amino-3-phenyl-1H-indole-2-carboxylic acid pyrindin-4-ylamide is obtained.

NMR (300 MHz, DMSO-d6): δ 5.05 (br, 2H), 6.70-6.74 (m, 2H), 7.24 (d, 1H), 7.33 (t, 1H), 7.41-7.50 (m, 6H), 8.41 (d, 2H), 9.95 (s, 1H), 11.16 (s, 1H).

According to Instructions 3, after the reaction of 5-amino-3-phenyl-1H-indole-2-carboxylic acid pyrindin-4-ylamide (33 mg, 0.10 mmol) with 4-tert-butylbenzenesulfonic acid chloride (28 mg, 0.12 mmol) and chromatographic purification (silica gel, hexane/ethyl acetate (0-100% ethyl acetate)), 17 mg (32%) of the product 5-(4-tert-butylbenzenesulfonylamino)-3-phenyl-1H-indole-2-carboxylic acid-pyrindin-4-ylamide is obtained.

NMR (300 MHz, DMSO-d6): δ 1.26 (s, 9H), 7.11 (dd, 1H), 7.18 (s, 1H), 7.31-7.61 (m, 12H), 8.43 (d, 2H), 9.87 (s, 1H), 10.14 (s, 1H), 12.00 (1H).

### Example 22

### 5-(4-tert-Butylbenzylamino)-3-phenyl-1H-indole-2-carboxylic acid pyridin-4-ylamide

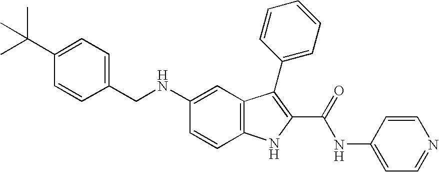

5-Amino-3-phenyl-1H-indole-2-carboxylic acid pyrindin-4-ylamide (270 mg, 0.82 mmol) and 4-tert-butylbenzaldehyde (146 mg, 0.90 mmol) are introduced into 34 ml of xylene, mixed with titanium tetraethylate (0.34 ml, 1.64 mmol), and refluxed for 9 hours. After removal of the solvent and after chromatographic purification of the residue (silica gel, hexane/ethyl acetate (0-100% ethyl acetate)), 130 mg (33%) of the product 5-{[1-(4-tert-butylphenyl)-methylidene]-amino-3-phenyl-1H-indole-2-carboxylic acid pyridin-4-ylamide is produced.

NMR (300 MHz, DMSO-d6): δ 1.30 (s, 9H), 7.38-7.63 (m, 12H), 7.88 (d, 2H), 8.44 (d, 2H), 8.69 (s, 1H), 10.17 (s, 1H), 12.10 (s, 1H).

5-{[1-(4-tert-Butylphenyl)-methylidene]-amino}-3-phenyl-1H-indole-2-carboxylic acid-pyridin-4-ylamide (70 mg, 0.15 mmol) is dissolved in 5 ml of methanol and mixed at 0° C. with sodium borohydride (44 mg, 1.14 mmol). The arrest of the reaction is carried out by adding water. After extraction with ethyl acetate, the combined organic phases are washed with saturated sodium chloride solution and dried on sodium sulfate. After removal of the solvent and after chromatographic purification of the residue (silica gel, hexane/ethyl acetate (0-100% ethyl acetate)), 50 mg (71%) of the product 5-(4-tert-butylbenzylamino)-3-phenyl-1H-indole-2-carboxylic acid pyridin-4-ylamide is produced.

NMR (300 MHz, DMSO-d6): δ 1.27 (s, 9H), 4.18 (d, 2H), 5.91 (t, 1H), 6.56 (d, 1H), 6.84 (dd, 1H), 7.20-7.45 (m, 10H), 7.48 (d, 2H), 8.43 (d, 2H), 9.90 (s, 1H), 11.56 (s, 1H).

### Example 23

### 5-(4-tert-Butylbenzoylamino)-3-phenyl-1H-indole-2-carboxylic acid-(2-dimethylaminoethyl)amide

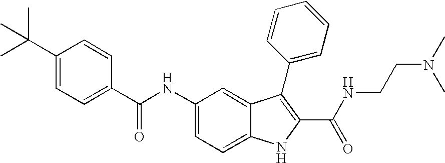

4-tert-Butylbenzoic acid (185 mg, 1.04 mmol) is introduced into 5 ml of DME and mixed at 0° C. with thionyl chloride (0.09 ml, 1.24 mmol). After 30 minutes, 5-amino-3-phenyl-1H-indole-2-carboxylic acid-(2-dimethylaminoethyl)amide (500 mg, 1.55 mmol) is added and then stirred for 20 hours at room temperature. The arrest of the reaction is carried out by adding 5 ml of a 10% aqueous citric acid solution, then the solution is brought to a basic range with saturated sodium bicarbonate solution. After extraction with ethyl acetate, the drying of the combined organic phases on sodium sulfate follows. After removal of the solvent under reduced pressure and chromatographic purification (silica gel, dichloromethane/methanol (0-10% methanol)), the yield is 22 mg (3%) of product.

NMR (300 MHz, DMSO-d6): δ 1.31 (s, 9H), 2.00 (s, 6H), 2.22 (t, 2H), 3.38 (q, 2H), 7.00 (t, 1H), 7.38-7.56 (m, 8H), 7.61 (dd, 1H), 7.88 (d, 2H), 7.93 (s, 1H), 10.08 (s, 1H), 11.70 (s, 1H).

### Example 24

### 5-(4-tert-Butylbenzenesulfonylamino)-3-(2-chlorophenyl)-1H-indole-2-carboxylic acid-(2-dimethylaminoethyl)amide

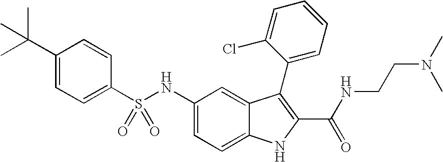

According to Instructions 2, after the reaction of 5-nitro-1H-indole-2-carboxylic acid ethyl ester (500 mg, 2.13 mmol) with ammonium formate (671 mg, 10.65 mmol) in the presence of palladium on carbon (50 mg), 330 mg (76%) of 5-amino-1H-indole-2-carboxylic acid ethyl ester is produced.

NMR (300 MHz, DMSO-d6): δ 1.32 (t, 3H), 4.30 (q, 2H), 4.70 (br, 2H), 6.64-6.72 (m, 2H), 6.83 (d, 1H), 7.25 (d, 1H), 11.40 (s, 1H).

According to Instructions 3, after the reaction of 5-amino-1H-indole-2-carboxylic acid ethyl ester (160 mg, 0.78 mmol) with 4-tert-butylbenzenesulfonic acid chloride (181 mg, 0.78 mmol) and chromatographic purification (silica gel, hexane/ethyl acetate (0-100% ethyl acetate)), 270 mg (86%) of the product 5-(4-tert-butylbenzenesulfonylamino)-1H-indole-2-carboxylic acid ethyl ester is obtained.

NMR (300 MHz, DMSO-d6): δ 1.25 (s, 9H), 1.32 (t, 3H), 4.32 (q, 2H), 7.02-7.08 (m, 2H), 7.30 (d, 1H), 7.35 (d, 1H), 7.52 (d, 2H), 7.62 (d, 2H), 9.97 (s, 1H), 11.35 (s, 1H).

According to Instructions 4, after the reaction of 5-(4-tert-butylbenzenesulfonylamino)-1H-indole-2-carboxylic acid ethyl ester (270 mg, 067 mmol) with N-bromosuccinimide (120 mg, 0.67 mmol) and chromatographic purification (silica gel, hexane/ethyl acetate (0-100% ethyl acetate)), 240 mg (75%) of the product 3-bromo-5-(4-tert-butylbenzenesulfonylamino)-1H-indole-2-carboxylic acid ethyl ester is obtained.

NMR (300 MHz, DMSO-d6): δ 1.25 (s, 9H), 1.32 (t, 3H), 4.34 (q, 2H), 7.12-7.18 (m, 2H), 7.35 (d, 1H), 7.52 (d, 2H), 7.65 (d, 2H), 10.10 (s, 1H), 12.1 (s, 1H).

According to Instructions 6, after the reaction of 3-bromo-5-(4-tert-butylbenzenesulfonylamino)-1H-indole-2-carboxylic acid ethyl ester (400 mg, 0.83 mmol) with 2-chlorophenylboronic acid (186 mg, 1.19 mmol) and chromatographic purification (silica gel, hexane/ethyl acetate (0-100% ethyl acetate)), 320 mg (70%) of the product 5-(4-tert-butylbenzenesulfonylamino)-3-(2-chlorophenyl)-1H-indole-2-carboxylic acid ethyl ester is obtained.

NMR (300 MHz, DMSO-d6): δ 1.02 (t, 3H), 1.25 (s, 9H), 4.10-4.20 (m, 2H), 6.82 (s, 1H), 7.14 (d, 1H), 7.22 (dd, 1H), 7.32-7.44 (m, 3H), 7.46-7.58 (m, 5H), 9.80 (s, 1H), 12.08 (s, 1H).

According to Instructions 5, after the reaction of (320 mg, 0.63 mmol) with 11.5 ml of a 1 M NaOH solution in ethanol/water (1/1), 290 mg (95%) of the product 5-(4-tert-butylbenzenesulfonylamino)-3-(2-chlorophenyl)-1H-indole-2-carboxylic acid is obtained.

NMR (300 MHz, DMSO-d6): δ 1.25 (s, 9H), 6.77 (d, 1H), 7.12 (d, 1H), 7.25 (dd, 1H), 7.32-7.44 (m, 3H), 7.48-7.56 (m, 5H), 9.80 (s, 1H), 11.90 (s, 1H), 12.75 (br, 1H).

According to Instructions 1, after the reaction of 5-(4-tert-butylbenzenesulfonyl-amino)-3-(2-chlorophenyl)-1H-indole-2-carboxylic acid (290 mg, 0.6 mmol) with N,N-dimethylethylenediamine (0.066 ml, 0.6 mmol) and chromatographic purification (silica gel, dichloromethane/methanol (0-20% methanol)), 180 mg (54%) of the product 5-(4-tert-butylbenzenesulfonylamino)-3-(2-chlorophenyl)-1H-indole-2-carboxylic acid-(2-dimethylaminoethyl)amide is obtained.

NMR (300 MHz, DMSO-d6): δ 1.25 (s, 9H), 1.96 (s, 6H), 2.19 (t, 2H), 3.18-3.23 (m, 2H), 6.73-6.74 (m, 2H), 7.05 (dd, 1H), 7.28 (dd, 1H), 7.35 (d, 1H), 7.40-7.53 (m, 6H), 7.59 (dd, 1H), 9.75 (s, 1H), 11.80 (s, 1H).

### Example 25

### 5-(4-tert-Butylbenzenesulfonylamino)-3-(3-chlorophenyl)-1H-indole-2-carboxylic acid-(2-dimethylaminoethyl)amide

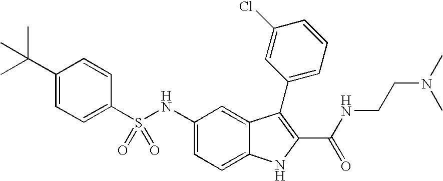

According to Instructions 6, after the reaction of 3-bromo-5-(4-tert-butylbenzenesulfonylamino)-1H-indole-2-carboxylic acid ethyl ester (400 mg, 0.83 mmol) with 3-chlorophenylboronic acid (186 mg, 1.19 mmol) and chromatographic purification (silica gel, hexane/ethyl acetate (0-100% ethyl acetate)), 300 mg (66%) of the product 5-(4-tert-butylbenzenesulfonylamino)-3-(3-chlorophenyl)-1H-indole-2-carboxylic acid ethyl ester is obtained.

NMR (300 MHz, DMSO-d6): δ 1.15 (t, 3H), 1.25 (s, 9H), 4.22 (q, 2H), 7.08 (s, 1H), 7.13 (dd, 1H), 7.20-7.28 (m, 1H), 7.34-7.45 (m, 4H), 7.52 (d, 2H), 7.60 (d, 2H), 9.92 (s, 1H), 12.05 (s, 1H).

According to Instructions 5, after the reaction of 5-(4-tert-butylbenzenesulfonyl-amino)-3-(3-chlorophenyl)-1H-indole-2-carboxylic acid ethyl ester (300 mg, 0.58 mmol) with 10.9 ml of a 1 M NaOH solution in ethanol/water (1/1), 240 mg (85%) of the product (4-tert-butylbenzenesulonylamino)-3-(3-chlorophenyl)-1H-indole-2-carboxylic acid is obtained.

NMR (300 MHz, DMSO-d6): δ 1.23 (s, 9H), 7.06 (s, 1H), 7.11 (dd, 1H), 7.25 (d, 1H), 7.34-7.45 (m, 4H), 7.51 (d, 2H), 7.10 (d, 2H), 9.89 (s, 1H), 11.92 (s, 1H), 12.20 (br, 1H).

According to Instructions 1, after the reaction of 5-(4-tert-butylbenzenesulfonyl-amino)-3-(3-chlorophenyl)-1H-indole-2-carboxylic acid (240 mg, 0.5 mmol) with N,N-dimethylethylenediamine (0.054 ml, 0.5 mmol) and chromatographic purification (silica gel, dichloromethane/methanol (0-20% methanol)), 90 mg (33%) of the product 5-(4-tert-butylbenzenesulonylamino)-3-(3-chlorophenyl)-1H-indole-2-carboxylic acid-(2-dimethylaminoethyl)amide is obtained.

NMR (300 MHz, DMSO-d6): δ 1.23 (s, 9H), 2.06 (s, 6H), 2.25 (t, 2H), 3.24 (q, 2H), 7.04-7.07 (m, 2H), 7.24 (d, 1H), 7.32-7.35 (m, 3H), 7.45-7.59 (m, 6H), 9.86 (s, 1H), 11.82 (s, 1H).

### Example 26

### 5-(4-tert-Butylbenzenesulfonylamino)-3-(4-chlorophenyl)-1H-indole-2-carboxylic acid-(2-dimethylaminoethyl)amide

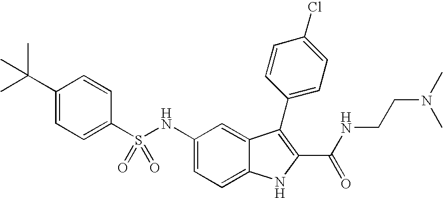

According to Instructions 6, after the reaction of 3-bromo-5-(4-tert-butylbenzenesulfonylamino)-1H-indole-2-carboxylic acid ethyl ester (500 mg, 1.05 mmol) with 4-chlorophenylboronic acid (236 mg, 1.5 mmol) and chromatographic purification (silica gel, hexane/ethyl acetate (0-100% ethyl acetate)), 396 mg (74%) of the product 5-(4-tert-butylbenzenesulfonylamino)-3-(4-chlorophenyl)-1H-indole-2-carboxylic acid-ethyl ester is obtained.

NMR (300 MHz, DMSO-d6): δ 1.15 (t, 3H), 1.25 (s, 9H), 4.22 (q, 2H), 7.07 (s, 1H), 7.14 (dd, 1H), 7.34 (d, 2H), 7.39 (d, 1H), 7.44-7.60 (m, 6H), 9.90 (s, 1H), 11.98 (s, 1H).

According to Instructions 5, after the reaction of the product 5-(4-tert-butylbenzenesulfonylamino)-3-(4-chlorophenyl)-1H-indole-2-carboxylic acid ethyl ester Azb SV 148 (396 mg, 0.77 mmol) with 14.5 ml of a 1 M NaOH solution in ethanol/water (1/1), 302 mg (81%) of the product 5-(4-tert-butylbenzenesulfonylamino)-3-(4-chlorophenyl)-1H-indole-2-carboxylic acid is obtained.

NMR (300 MHz, DMSO-d6): δ 1.23 (s, 9H), 7.14 (s, 1H), 7.19 (dd, 1H), 7.38-7.48 (m, 3H), 7.47 (d, 2H), 7.53 (d, 2H), 7.60 (d, 2H), 9.89 (s, 1H), 11.90 (s, 1H), 12.90 (br, 1H).

According to Instructions 1, after the reaction of 5-(4-tert-butylbenzenesulfonyl-amino)-3-(4-chlorophenyl)-1H-indole-2-carboxylic acid (335 mg, 0.69 mmol) with N,N-dimethylethylenediamine (0.08 ml, 0.69 mmol) and chromatographic purification (silica gel, dichloromethane/methanol (0-20% methanol)), 112 mg (29%) of the product 5-(4-tert-butylbenzenesulonylamino)-3-(4-chlorophenyl)-1H-indole-2-carboxylic acid-(2-dimethylaminoethyl)amide is obtained.

NMR (300 MHz, DMSO-d6): δ 1.24 (s, 9H), 2.03 (s, 6H), 2.23 (t, 2H), 3.23 (q, 2H), 7.01-7.04 (m, 2H), 7.17 (t, 1H), 7.29-7.35 (m, 3H), 7.51-7.60 (m, 6H), 9.83 (s, 1H), 11.78 (s, 1H).

### Example 27

### 5-(4-tert-Butylbenzenesulfonylamino)-3-(2,4-dichlorophenyl)-1H-indole-2-carboxylic acid-(2-dimethylaminoethyl)amide

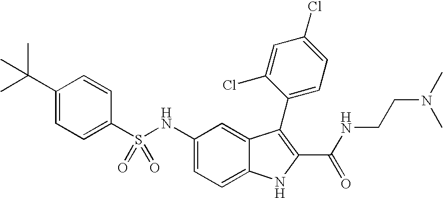

According to Instructions 6, after the reaction of 3-bromo-5-(4-tert-butylbenzenesulfonylamino)-1H-indole-2-carboxylic acid ethyl ester (400 mg, 0.83 mmol) with 2,4-dichlorophenylboronic acid (227 mg, 1.19 mmol) and chromatographic purification (silica gel, hexane/ethyl acetate (0-100% ethyl acetate)), 370 mg (81%) of the product 5-(4-tert-butylbenzenesulonylamino)-3-(2,4-dichlorophenyl)-1H-indole-2-carboxylic acid ethyl ester is obtained.

NMR (300 MHz, DMSO-d6): δ 1.15 (t, 3H), 1.23 (s, 9H), 4.22 (q, 2H), 6.80 (d, 1H), 7.14 (dd, 1H), 7.30 (d, 1H), 7.40 (d, 1H), 7.42-7.56 (m, 5H), 7.70 (d, 1H), 9.89 (s, 1H), 12.12 (s, 1H).

According to Instructions 5, after the reaction of 5-(4-tert-butylbenzenesulfonyl-amino)-3-(2,4-dichlorophenyl)-1H-indole-2-carboxylic acid ethyl ester (370 mg, 0.68 mmol) with 12.6 ml of a 1 M NaOH solution in ethanol/water (1/1), 330 mg (94%) of the product 5-(4-tert-butylbenzenesulfonylamino)-3-(2,4-dichlorophenyl)-1H-indole-2-carboxylic acid is obtained.

NMR (300 MHz, DMSO-d6): δ 1.23 (s, 9H), 6.78 (d, 1H), 7.12 (dd, 1H), 7.30 (d, 1H), 7.38 (d, 1H), 7.42-7.58 (m, 5H), 7.70 (d, 1H), 9.85 (s, 1H), 12.02 (s, 1H).

According to Instructions 1, after the reaction of 5-(4-tert-butylbenzenesulfonyl-amino)-3-(2,4-dichlorophenyl)-1H-indole-2-carboxylic acid (330 mg, 0.64 mmol) with N,N-dimethylethylenediamine (0.07 ml, 0.64 mmol) and chromatographic purification (silica gel, dichloromethane/methanol (0-20% methanol)), 170 mg (45%) of the product 5-(4-tert-butylbenzenesulfonylamino)-3-(2,4-dichlorophenyl)-1H-indole-2-carboxylic acid-(2-dimethylaminoethyl)amide is obtained.

NMR (300 MHz, DMSO-d6): δ 1.24 (s, 9H), 2.00 (s, 6H), 2.21 (t, 2H), 3.21-3.24 (m, 2H), 6.73 (d, 1H), 6.85 (t, 1H), 7.06 (dd, 1H), 7.31-7.39 (m, 2H), 7.47-7.56 (m, 5H), 7.77 (d, 1H), 9.79 (s, 1H), 11.87 (s, 1H).

### Example 28

### 5-(4-tert-Butylbenzenesulfonylamino)-3-(2-methylphenyl)-1H-indole-2-carboxylic acid-(2-dimethylaminoethyl)amide

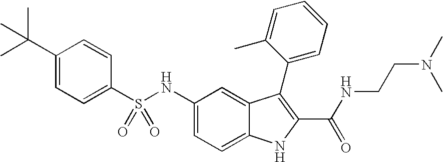

According to Instructions 6, after the reaction of 3-bromo-5-(4-tert-butylbenzenesulfonylamino)-1H-indole-2-carboxylic acid ethyl ester (400 mg, 0.83 mmol) with o-toluoylboronic acid (161 mg, 1.19 mmol) and chromatographic purification (silica gel, hexane/ethyl acetate (0-100% ethyl acetate)), 250 mg (55%) of the product 5-(4-tert-butylbenzenesulfonylamino)-3-(2-methylphenyl)-1H-indole-2-carboxylic acid ethyl ester is obtained.

NMR (300 MHz, DMSO-d6): δ 1.02 (s, 3H), 1.25 (s, 9H), 1.88 (s, 3H), 4.11 (q, 2H), 6.70 (d, 1H), 7.02 (d, 1H), 7.12-7.24 (m, 2H), 7.28-7.32 (m, 2H), 7.38 (d, 1H), 7.45-7.53 (m, 4H), 9.80 (s, 1H), 11.90 (s, 1H).

According to Instructions 5, after the reaction of the product 5-(4-tert-butylbenzenesulfonylamino)-3-(2-methylphenyl)-1H-indole-2-carboxylic acid ethyl ester (250 mg, 0.51 mmol) with 9.4 ml of a 1 M NaOH solution in ethanol/water (1/1), the product 5-(4-tert-butylbenzenesulfonylamino)-3-(2-methylphenyl)-1H-indole-2-carboxylic acid is obtained in a quantitative yield.

NMR (300 MHz, DMSO-d6): δ 1.23 (s, 9H), 1.88 (s, 3H), 6.68 (s, 1H), 7.00 (d, 1H), 7.12 (dd, 1H), 7.14-7.20 (m, 1H), 7.24-7.28 (m, 2H), 7.35 (d, 1H), 7.50 (m, 4H), 9.73 (s, 1H), 11.78 (s, 1H).

According to Instructions 1, after the reaction of 5-(4-tert-butylbenzenesulfonyl-amino)-3-(2-methylphenyl)-1H-indole-2-carboxylic acid (320 mg, 0.69 mmol) with N,N-dimethylethylenediamine (0.076 ml, 0.69 mmol) and chromatographic purification (silica gel, dichloromethane/methanol (0-20% methanol)), 140 mg (38%) of the product 5-(4-tert-butylbenzenesulonylamino)-3-(2-methylphenyl)-1H-indole-2-carboxylic acid-(2-dimethylaminoethyl)amide is obtained.

NMR (300 MHz, DMSO-d6): δ 1.25 (s, 9H), 1.88 (s, 3H), 1.89 (s, 6H), 2.08-2.12 (m, 2H), 3.13-3.18 (m, 2H), 6.42 (t, 1H), 6.62 (d, 1H), 7.04-7.10 (m, 2H), 7.24-7.30 (m, 1H), 7.34-7.37 (m, 3H), 7.49 (2d, 4H), 9.70 (s, 1H), 11.71 (s, 1H).

### Example 29

### 5-(4-tert-Butylbenzenesulfonylamino)-3-(4-methylphenyl)-1H-indole-2-carboxylic acid-(2-dimethylaminoethyl)amide

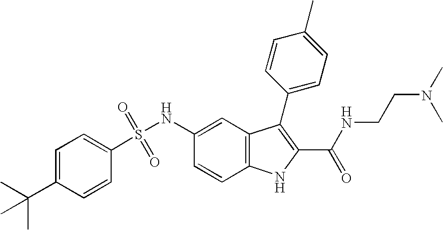

According to Instructions 6, after the reaction of 3-bromo-5-(4-tert-butylbenzenesulfonylamino)-1H-indole-2-carboxylic acid ethyl ester (500 mg, 1.05 mmol) with p-toluoylboronic acid (204 mg, 1.5 mmol) and chromatographic purification (silica gel, hexane/ethyl acetate (0-100% ethyl acetate)), 420 mg (82%) of the product 5-(4-tert-butylbenzenesulfonylamino)-3-(4-methylphenyl)-1H-indole-2-carboxylic acid ethyl ester is obtained.

NMR (300 MHz, DMSO-d6): δ 1.14 (t, 3H), 1.24 (s, 9H), 2.38 (s, 3H), 4.22 (q, 2H), 7.05 (d, 1H), 7.10 (dd, 1H), 7.18 (d, 2H), 7.22 (d, 2H), 7.36 (d, 1H), 7.52-7.60 (m, 4H), 9.84 (s, 1H), 11.82 (s, 1H).

According to Instructions 5, after the reaction of 5-(4-tert-butylbenzenesulfonyl-amino)-3-(4-methylphenyl)-1H-indole-2-carboxylic acid ethyl ester (420 mg, 0.86 mmol) with 16 ml of a 1 M NaOH solution in ethanol/water (1/1), 340 mg (85%) of the product 5-(4-tert-butylbenzenesulfonylamino)-3-(4-methylphenyl)-1H-indole-2-carboxylic acid is obtained.

NMR (300 MHz, DMSO-d6): 6.1.23 (s, 9H), 2.34 (s, 3H), 6.98-7.04 (m, 2H), 7.12 (d, 2H), 7.21 (d, 2H), 7.30 (d, 1H), 7.50-7.60 (m, 4H), 9.75 (s, 1H), 11.48 (s, 1H).

According to Instructions 1, after the reaction of 5-(4-tert-butylbenzenesulfonylamino)-3-(4-methylphenyl)-1H-indole-2-carboxylic acid (340 mg, 0.74 mmol) with N,N-dimethylethylenediamine (0.082 ml, 0.74 mmol) and chromatographic purification (silica gel, amine phase, dichloromethane/methanol (0-20% methanol)), 260 mg (66%) of the product 5-(4-tert-butylbenzenesulfonylamino)-3-(4-methylphenyl)-1H-indole-2-carboxylic acid-(2-dimethylaminoethyl)amide is obtained.

NMR (300 MHz, DMSO-d6): δ 1.25 (s, 9H), 1.99 (s, 6H), 2.19 (t, 2H), 2.38 (s, 3H), 3.21 (q, 2H), 6.94-7.03 (m, 3H), 7.17 (d, 2H), 7.26-7.33 (m, 3H), 7.51 (d, 2H), 7.56 (d, 2H), 9.76 (s, 1H), 11.67 (s, 1H).

### Example 30

### 5-(4-tert-Butylbenzenesulfonylamino)-3-(4-methylphenyl)-1H-indole-2-carboxylic acid pyridin-4ylamide

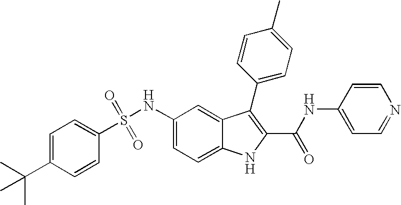

According to Instructions 1, after the reaction of 5-(4-tert-butylbenzenesulfonyl-amino)-3-(4-methylphenyl)-1H-indole-2-carboxylic acid (170 mg, 0.37 mmol) with 4-aminopyridine (35 mg, 0.37 mmol), 130 mg (65%) of the product is obtained.

NMR (300 MHz, DMSO-d6): δ 1.25 (s, 9H), 2.36 (s, 3H), 7.09 (dd, 1H), 7.16-7.25 (m, 5H), 7.38 (d, 1H), 7.49 (d, 2H), 7.53 (d, 2H), 7.59 (d, 2H), 8.43 (d, 2H), 9.85 (s, 1H), 10.09 (s, 1H), 11.94 (s, 1H).

### Example 31

### 5-(4-tert-Butylbenzenesulfonylamino)-3-(4-methoxyphenyl)-1H-indole-2-carboxylic acid-(2-dimethylaminoethyl)amide

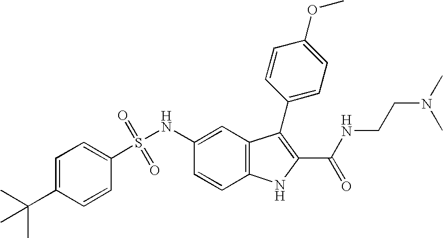

According to Instructions 6, after the reaction of 3-bromo-5-(4-tert-butylbenzenesulfonylamino)-1H-indole-2-carboxylic acid ethyl ester (500 mg, 1.05 mmol) with 4-methoxyphenylboronic acid (228 mg, 1.5 mmol) and chromatographic purification (silica gel, hexane/ethyl acetate (0-100% ethyl acetate)), 400 mg (75%) of the product 5-(4-tert-butylbenzenesulfonylamino)-3-(4-methoxyphenyl)-1H-indole-2-carboxylic acid ethyl ester is obtained.

NMR (300 MHz, DMSO-d6): δ 1.14 (t, 3H), 1.24 (s, 9H), 3.82 (s, 3H), 4.22 (q, 2H), 6.96 (d, 2H), 7.08-7.12 (m, 2H), 7.24 (d, 2H), 7.35 (d, 1H), 7.53 (d, 2H), 7.64 (d, 2H), 9.85 (s, 1H), 11.80 (s, 1H).

According to Instructions 5, after the reaction of 5-(4-tert-butylbenzenesulfonyl-amino)-3-(4-methoxyphenyl)-1H-indole-2-carboxylic acid ethyl ester (420 mg, 0.86 mmol) with 15 ml of a 1 M NaOH solution in ethanol/water (1/1), the product 5-(4-tert-butylbenzenesulfonylamino)-3-(4-methoxyphenyl)-1H-indole-2-carboxylic acid is obtained in a quantitative yield.

NMR (300 MHz, DMSO-d6): δ 1.24 (s, 9H), 3.82 (s, 3H), 6.96 (d, 2H), 7.08-7.12 (m, 2H), 7.22 (d, 2H), 7.34 (d, 1H), 7.52 (d, 2H), 7.60 (d, 2H), 9.80 (s, 1H), 11.70 (s, 1H), 12.25 (br, 1H).

According to Instructions 1, after the reaction of 5-(4-tert-butylbenzenesulfonyl-amino)-3-(4-methoxyphenyl)-1H-indole-2-carboxylic acid (390 mg, 0.82 mmol) with N,N-dimethylethylenediamine (0.091 ml, 0.82 mmol) and chromatographic purification (silica gel, dichloromethane/methanol (0-20% methanol)), 200 mg (44%) of the product 5-(4-tert-butylbenzenesulfonylamino)-3-(4-methoxyphenyl)-1H-indole-2-carboxylic acid-(2-dimethylaminoethyl)amide is obtained.

NMR (300 MHz, DMSO-d6): δ 1.25 (s, 9H), 2.01 (s, 6H), 2.22 (t, 2H), 3.22 (q, 2H), 3.83 (s, 3H), 6.93 (t, 1H), 6.98-6.99 (m, 2H), 7.03 (d, 2H), 7.21 (d, 2H), 7.31 (d, 1H), 7.52 (d, 2H), 7.56 (d, 2H), 9.77 (s, 1H), 11.63 (s, 1H).

### Example 32

### 5-(4-tert-Butylbenzenesulfonylamino)-3-pyridin-3-yl-1H-indole-2-carboxylic acid-(2-dimethylaminoethyl)amide

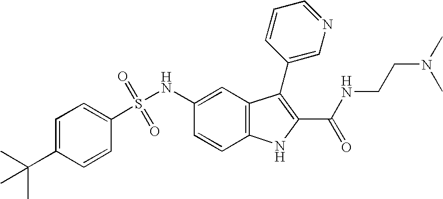

According to Instructions 6, after the reaction of 3-bromo-5-(4-tert-butylbenzenesulfonylamino)-1H-indole-2-carboxylic acid ethyl ester (500 mg, 1.05 mmol) with pyridine-3-boronic acid (184 mg, 1.5 mmol) and chromatographic purification (silica gel, hexane/ethyl acetate (0-100% ethyl acetate)), 280 mg (56%) of the product 5-(4-tert-butylbenzenesulfonylamino)-3-pyridin-3-yl-1H-indole-2-carboxylic acid ethyl ester is obtained.

NMR (300 MHz, DMSO-d6): δ 1.14 (t, 3H), 1.24 (s, 9H), 4.21 (q, 2H), 7.05 (d, 1H), 7.12 (dd, 1H), 7.40-7.60 (m, 6H), 7.72 (m, 1H), 8.50 (d, 1H), 8.58 (dd, 1H), 9.90 (s, 11H), 12.10 (s, 11H).

According to Instructions 5, after the reaction of 5-(4-tert-butylbenzenesulfonyl-amino)-3-pyridin-3-yl-1H-indole-2-carboxylic acid ethyl ester (280 mg, 0.59 mmol) with 11 ml of a 1 M NaOH solution in ethanol/water (1/1), 260 mg (98%) of the product 5-(4-tert-butylbenzenesulonylamino)-3-pyridin-3-yl-1H-indole-2-carboxylic acid is obtained.

NMR (300 MHz, DMSO-d6): δ 1.22 (s, 9H), 7.08-7.12 (m, 2H), 7.40 (d, 1H), 7.52 (d, 2H), 7.58 (d, 2H), 7.70-7.76 (m, 1H), 8.05 (d, 1H), 8.68 (dd, 1H), 8.72 (d, 1H), 9.95 (s, 1H), 12.12 (s, 1H).

According to Instructions 1, after the reaction of 5-(4-tert-butylbenzenesulfonyl-amino)-3-pyridin-3-yl-1H-indole-2-carboxylic acid (260 mg, 0.58 mmol) with N,N-dimethylethylenediamine (0.064 ml, 0.58 mmol) and chromatographic purification (silica gel, dichloromethane/methanol (0-20% methanol)) as well as subsequent recrystallization from dichloromethane, 130 mg (43%) of the product 5-(4-tert-butylbenzenesulfonylamino)-3-pyridin-3-yl-1H-indole-2-carboxylic acid-(2-dimethylaminoethyl)amide is obtained.

NMR (300 MHz, DMSO-d6): δ 1.25 (s, 9H), 2.06 (s, 6H), 2.25 (t, 2H), 3.24 (q, 2H), 7.04-7.08 (m, 2H), 7.36 (d, 1H), 7.44-7.58 (m, 6H), 7.68-7.71 (m, 1H), 8.46 (d, 1H), 8.56 (dd, 1H), 9.82 (s, 1H), 11.85 (s, 1H).

### Example 33

### 5-(4-tert-Butylbenzenesulfonylamino)-3-(4-hydroxyphenyl)-1H-indole-2-carboxylic acid-(2-dimethylaminoethyl)amide

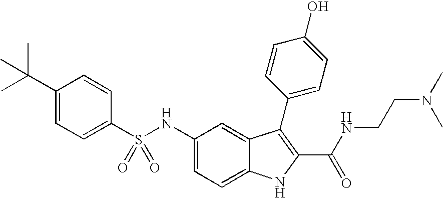

5-(4-tert-Butylbenzenesulfonylamino)-3-(4-methoxyphenyl)-1H-indole-2-carboxylic acid-(2-dimethylaminoethyl)amide (180 mg, 0.33 mmol) is mixed with 9.70 ml of a 1 M boron tribromide solution in dichloromethane (9.70 mmol) and stirred for 20 hours at room temperature. The arrest of the reaction is carried out by adding saturated sodium bicarbonate solution. After extraction with ethyl acetate, the combined organic phases are washed with 2N sodium hydroxide solution and saturated sodium chloride solution. After drying on sodium sulfate, after removal of the solvent as well as after chromatographic purification (silica gel, dichloromethane/methanol (0-50% methanol)), 60 mg (34% of theory) of the product is obtained.

NMR (300 MHz, DMSO-d6): δ 1.26 (s, 9H), 2.00 (s, 6H), 2.19 (t, 2H), 3.23 (q, 2H), 6.81-6.87 (m, 3H), 6.94 (s, 1H), 6.99-7.08 (m, 3H), 7.31 (d, 1H), 7.50-7.56 (AA′BB′, 4H), 9.58 (s, 1H), 9.75 (s, 1H), 11.58 (s, 1H).

### Example 34

### 5-(4-tert-Butylbenzenesulfonylamino)-3-(3-fluorophenyl)-1H-indole-2-carboxylic acid-(2-dimethylaminoethyl)amide

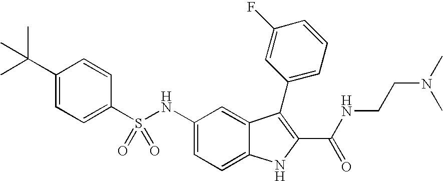 5-(4-tert-Butylbenzenesulfonylamino)-3-(3-fluorophenyl)-1H-indole-2-carboxylic acid ethyl ester: According to Instructions 6, after the reaction of 3-bromo-5-(4-tert-butylbenzenesulfonylamino)-1H-indole-2-carboxylic acid ethyl ester (400 mg, 0.83 mmol) with 3-fluorophenylboronic acid (167 mg, 1.19 mmol) and chromatographic purification (silica gel, hexane/ethyl acetate-(0-100% ethyl acetate)), 290 mg (71%) of the product is obtained.

NMR (300 MHz, DMSO-d6): δ 1.17 (t, 3H), 1.24 (s, 9H), 4.23 (q, 2H), 7.05-7.65 (m, 11H), 9.88 (s, 1H), 12.01 (s, 1H)

### 5-(4-tert-Butylbenzenesulonylamino)-3-(3-fluorophenyl)-1H-indole-2-carboxylic acid

According to Instructions 5, after the reaction of 5-(4-tert-butylbenzenesulfonyl-amino)-3-(3-fluorophenyl)-1H-indole-2-carboxylic acid ethyl ester (280 mg, 0.59 mmol) with 9.1 ml of a 1 M NaOH solution in ethanol/water (1/1), the product is obtained in a quantitative yield.

NMR (300 MHz, DMSO-d6): δ 1.24 (s, 9H), 6.98-7.21 (m, 5H), 7.34-7.46 (m, 6H), 9.86 (s, 1H), 11.89 (s, 1H), 12.90 (br, 1H).

5-(4-tert-Butylbenzenesulfonylamino)-3-(3-fluorophenyl)-1H-indole-2-carboxylic acid-(2-dimethylaminoethyl)amide: According to Instructions 1, after the reaction of 5-(4-tert-butylbenzenesulfonylamino)-3-(3-fluorophenyl)-1H-indole-2-carboxylic acid (240 mg, 0.51 mmol) with N,N-dimethylethylenediamine (0.056 ml, 0.51 mmol) and chromatographic purification (silica gel, dichloromethane/methanol (0-30% methanol)), 160 mg (59%) of the product is obtained.

NMR (300 MHz, DMSO-d6): δ 1.24 (s, 9H), 2.06 (s, 6H), 2.26 (t, 2H), 3.25 (q, 2H), 7.04-7.13 (m, 4H), 7.19-7.30 (m, 2H), 7.35 (d, 1H), 7.36-7.59 (m, 5H), 9.81 (s, 1H), 11.79 (s, 1H).

### Example 35

### 5-(4-tert-Butylbenzenesulfonylamino)-3-phenyl-1H-indole-2-carboxylic acid-(2-hydroxypropyl)amide

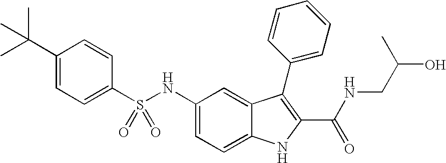 [0551] 5-(4-tert-Butylbenzenesulfonylamino)-3-phenyl-1H-indole-2-carboxylic acid ethyl ester:

According to Instructions 6, after the reaction of 3-bromo-5-(4-tert-butylbenzene-sulfonylamino)-1H-indole-2-carboxylic acid ethyl ester (2.0 g, 4.15 mmol) with phenylboronic acid (725 mg, 5.9 mmol) and chromatographic purification (silica gel, hexane/ethyl acetate (0-50% ethyl acetate)), 1.35 g (68%) of the product is obtained.

NMR (300 MHz, DMSO-d6): δ 1.17 (t, 3H), 1.25 (s, 9H), 4.19 (q, 2H), 7.07 (d, 1H), 7.12 (dd, 1H), 7.23-7.44 (m, 6H), 7.50-7.59 (m, 4H), 9.85 (s, 1H), 11.90 (s, 1H).

5-(4-tert-Butylbenzenesulfonylamino)-3-phenyl-1H-indole-2-carboxylic acid: According to Instructions 5, after the reaction of 5-(4-tert-butylbenzenesulfonyl-amino)-3-phenyl-1H-indole-2-carboxylic acid ethyl ester (1.35 g, 2.83 mmol) with 55 ml of a 1 M NaOH solution in ethanol/water (1/1), 1.17 g (92%) of the product is obtained.

NMR (300 MHz, DMSO-d6): δ 1.25 (s, 9H), 7.05 (d, 1H), 7.09 (dd, 1H), 7.27-7.43 (m, 6H), 7.55 (d, 2H), 7.60 (d, 2H), 9.82 (s, 1H), 11.80 (s, 1H), 12.5 (br, 1H).

5-(4-tert-Butylbenzenesulfonylamino)-3-phenyl-1H-indole-2-carboxylic acid-(2-hydroxy-propyl)amide: According to Instructions 1, after the reaction of 5-(4-tert-butylbenzene-sulfonylamino)-3-phenyl-1H-indole-2-carboxylic acid (199 mg, 0.44 mmol) with 1-amino-2-propanol (0.035 ml, 0.44 mmol) and preparative thin-layer chromatography (silica gel, dichloromethane/methanol 95:5), 33 mg (15%) of the product is obtained (AP 3795).

NMR (300 MHz, DMSO-d6): δ 0.94 (d, 3H), 1.25 (s, 9H), 2.99-3.23 (m, 2H), 3.54-3.66 (m, 1H), 4.60 (d, 1H), 7.02-7.04 (m, 2H), 7.16 (t, 1H), 7.28-7.51 (m, 6H), 7.52 (d, 2H), 7.57 (d, 2H), 9.79 (s, 1H), 11.71 (s, 1H).

### Example 36

### 5-(4-tert-Butylbenzenesulfonylamino)-3-phenyl-1H-indole-2-carboxylic acid-(2-methoxyethyl)amide

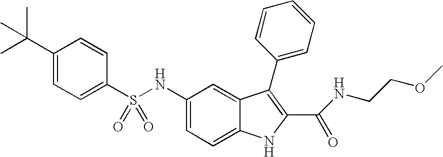

According to Instructions 1, after the reaction of 5-(4-tert-butylbenzene-sulfonylamino)-3-phenyl-1H-indole-2-carboxylic acid (199 mg, 0.44 mmol) with 2-methoxyethylamine (0.039 ml, 0.44 mmol) and preparative thin-layer chromatography (silica gel, dichloromethane/methanol 95:5), 20 mg (9%) of the product is obtained.

NMR (300 MHz, DMSO-d6): δ 1.25 (s, 9H), 3.16 (s, 3H), 3.31 (m, 4H), 7.02-7.05 (m, 2H), 7.20 (br, 1H), 7.30-7.48 (m, 6H), 7.52 (d, 2H), 7.57 (d, 2H), 9.79 (s, 1H), 11.71 (s, 1H).

### Example 37

### 5-(4-tert-Butylbenzenesulfonylamino)-3-phenyl-1H-indole-2-carboxylic acid-(2-hydroxyethyl)amide

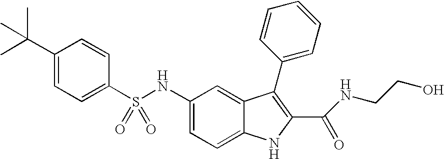

According to Instructions 1, after the reaction of 5-(4-tert-butylbenzene-sulfonylamino)-3-phenyl-1H-indole-2-carboxylic acid (199 mg, 0.44 mmol) with ethanolamine (0.027 ml, 0.44 mmol) and preparative thin-layer chromatography (silica gel, dichloromethane/methanol 95:5), 23 mg (11%) of the product is obtained.

NMR (300 MHz, DMSO-d6): δ 1.25 (s, 9H), 3.22 (q, 2H), 3.35-3.41 (m, 2H), 4.61 (t, 1H), 7.02-7.07 (m, 2H), 7.30-7.47 (m, 7H), 7.52 (d, 2H), 7.57 (d, 2H), 9.79 (s, 1H), 11.69 (s, 1H).

### Example 38

### 5-(4-tert-Butylbenzenesulfonylamino)-3-phenyl-1H-indole-2-carboxylic acid-(2-hydroxy-1-methylethyl)amide

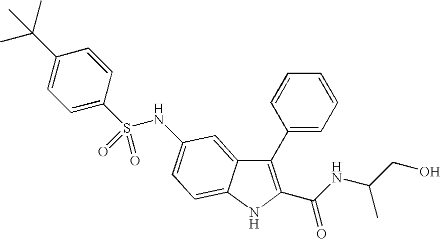

According to Instructions 1, after the reaction of 5-(4-tert-butylbenzene-sulfonylamino)-3-phenyl-1H-indole-2-carboxylic acid (199 mg, 0.44 mmol) with 2-amino-propanol (0.035 ml, 0.44 mmol) and preparative thin-layer chromatography (silica gel, dichloromethane/methanol 95:5), 32 mg (14%) of the product is obtained.

NMR (300 MHz, DMSO-d6): δ 0.96 (d, 3H), 1.25 (s, 9H), 3.16-3.33 (m, 2H), 3.87-3.96 (m, 1H), 4.63 (t, 1H), 6.95-7.06 (m, 3H), 7.28-7.47 (m, 6H), 7.52 (d, 2H), 7.57 (d, 2H), 9.79 (s, 1H), 11.71 (s, 1H).

### Example 39

### 5-(4-tert-Butylbenzenesulfonylamino)-3-phenyl-1H-indole-2-carboxylic acid-(2-acetylaminoethyl)amide

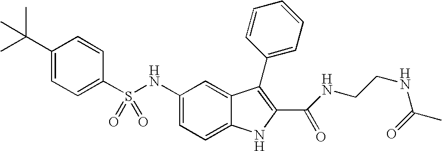

According to Instructions 1, after the reaction of 5-(4-tert-butylbenzene-sulfonylamino)-3-phenyl-1H-indole-2-carboxylic acid (199 mg, 0.44 mmol) with N-acetylethylenediamine (0.047 ml, 0.44 mmol) and preparative thin-layer chromatography (silica gel, dichloromethane/methanol 95:5), 42 mg (18%) of the product is obtained.

NMR (300 MHz, DMSO-d6): δ 1.25 (s, 9H), 1.76 (s, 3H), 3.07-3.10 (m, 2H), 3.17-3.26 (m, 2H), 7.03 (dd, 1H), 7.08 (s, 1H), 7.27-7.49 (m, 7H), 7.52 (d, 2H), 7.57 (d, 2H), 7.80 (t, 1H), 9.79 (s, 1H), 11.66 (s, 11H).

### Example 40

### 5-(4-tert-Butylbenzenesulfonylamino)-3-phenyl-1H-indole-2-carboxylic acid-(tetrahydropyran-4-yl)amide

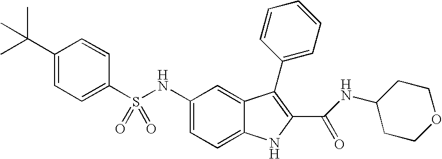

According to Instructions 1, after the reaction of 5-(4-tert-butylbenzene-sulfonylamino)-3-phenyl-1H-indole-2-carboxylic acid (199 mg, 0.44 mmol) with 4-aminotetrahydropyran (45 mg, 0.44 mmol) and preparative thin-layer chromatography (silica gel, dichloromethane/methanol 95:5), 45 mg (19%) of the product is obtained.

NMR (300 MHz, DMSO-d6): δ 1.13-1.44 (m, 1H), 1.66-1.70 (m, 2H), 3.30-3.37 (m, 2H), 3.69-3.73 (m, 2H), 3.84-3.89 (m, 1H), 7.03 (dd, 1H), 7.09 (d, 1H), 7.29-7.47 (m, 7H), 7.52 (d, 2H), 7.58 (d, 2H), 9.80 (s, 1H), 11.73 (s, 1H).

### Example 41

### 5-(4-tert-Butylbenzenesulfonylamino)-3-phenyl-1H-indole-2-carboxylic acid-(1-methylpiperidin-4-yl)amide

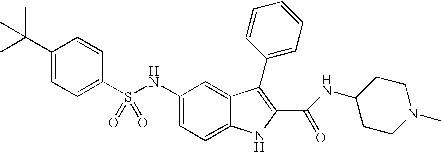

According to Instructions 1, after the reaction of 5-(4-tert-butylbenzene-sulfonylamino)-3-phenyl-1H-indole-2-carboxylic acid (199 mg, 0.44 mmol) with 4-amino-1-methylpiperidine (51 mg, 0.44 mmol) and chromatographic purification, 154 mg (64%) of the product is obtained.

NMR (300 MHz, DMSO-d6): δ 1.25 (s, 9H), 1.37-1.53 (br, 2H), 1.85-1.91 (m, 2H), 2.60 (s, 3H), 2.69-2.89 (br, 2H), 3.07-3.10 (br, 2H), 3.87-3.89 (br, 1H), 7.03 (dd, 1H), 7.12 (s, 1H), 7.29-7.47 (m, 7H), 7.52 (d, 2H), 7.57 (d, 2H), 9.82 (s, 1H), 11.71 (s, 1H).

### Example 42

### 5-(4-tert-Butylbenzenesulfonylamino)-3-(4-N,N-dimethylaminophenyl)-1H-indole-2-carboxylic acid-(2-dimethylaminoethyl)amide

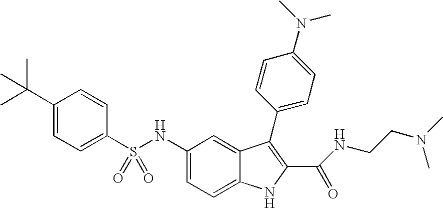 5-(4-tert-Butylbenzenesulfonylamino)-3-(4-N,N-dimethylaminophenyl)-1H-indole-2-carboxylic acid ethyl ester: According to Instructions 6, after the reaction of 3-bromo-5-(4-tert-butylbenzenesulfonylamino)-1H-indole-2-carboxylic acid ethyl ester (400 mg, 0.83 mmol) with 4-N,N-dimethylaminophenylboronic acid (196 mg, 1.19 mmol) and chromatographic purification (silica gel, hexane/ethyl acetate (0-100% ethyl acetate)), 90 mg (21%) of the product is obtained.

NMR (300 MHz, DMSO-d6): δ 1.23 (t, 3H), 1.25 (s, 9H), 2.96 (s, 6H), 4.20 (q, 2H), 6.74 (d, 2H), 7.06-7.17 (m, 4H), 7.34 (d, 1H), 7.53 (d, 2H), 7.58 (d, 2H), 9.81 (s, 1H), 11.69 (s, 1).

5-(4-tert-Butylbenzenesulfonylamino)-3-(4-N,N-dimethylaminophenyl)-1H-indole-2-carboxylic acid: According to Instructions 5, after the reaction of 5-(4-tert-butylbenzenesulfonylamino)-3-(4-N,N-dimethylaminophenyl)-1H-indole-2-carboxylic acid ethyl ester (90 mg, 0.59 mmol) with 3.2 ml of a 1 M NaOH solution in ethanol/water (1/1), the product is obtained in a quantitative yield.

5-(4-tert-Butylbenzenesulfonylamino)-3-(4-N,N-dimethylaminophenyl)-1H-indole-2-carboxylic acid-(2-dimethylaminoethyl)amide: According to Instructions 1, after the reaction of 5-(4-tert-butylbenzenesulfonylamino)-3-(4, N,N-dimethylaminophenyl-1H-indole-2-carboxylic acid (220 mg, 0.45 mmol) with N,N-dimethylethylenediamine (0.049 ml, 0.45 mmol) and chromatographic purification (silica gel, dichloromethane/methanol (0-30% methanol)), 22 mg (9%) of the product is obtained.

NMR (300 MHz, DMSO-d6): δ 1.24 (s, 9H), 2.05 (s, 6H), 2.27 (t, 2H), 2.97 (s, 6H), 3.24 (q, 2H), 6.81 (d, 2H), 6.89 (t, 1H), 6.98-7.01 (m, 2H), 7.11 (d, 2H), 7.29 (d, 1H), 7.50-7.57 (AA′BB′, 4H), 9.74 (s, 1H), 11.54 (s, 1H).

### Example 43

### 5-(4-tert-Butylbenzenesulfonylamino)-3-(3-methoxyphenyl)-1H-indole-2-carboxylic acid-(2-dimethylaminoethyl)amide

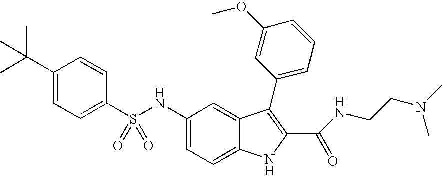 5-(4-tert-Butylbenzenesulonylamino)-3-(3-methoxyphenyl)-1H-indole-2-carboxylic acid ethyl ester: According to Instructions 6, after the reaction of 3-bromo-5-(4-tert-butylbenzenesulfonylamino)-1H-indole-2-carboxylic acid ethyl ester (400 mg, 0.83 mmol) with 3-methoxyphenylboronic acid (181 mg, 1.19 mmol) and chromatographic purification (silica gel, hexane/ethyl acetate (0-100% ethyl acetate)), 250 mg (60%) of the product is obtained.

NMR (300-MHz, DMSO-d6): δ 1.16 (t, 3H), 1.24 (s, 9H), 3.79 (s, 3H), 4.20 (q, 2H), 6.84 (d, 1H), 6.92-6.96 (m, 2H), 7.11-7.14 (m, 2H), 7.30-7.42 (m, 2H), 7.51 (d, 2H), 7.58 (d, 2H), 9.88 (s, 1H), 11.90 (s, 1H).

5-(4-tert-Butylbenzenesulfonylamino)-3-(3-methoxyphenyl)-1H-indole-2-carboxylic acid: According to Instructions 5, after the reaction of 5-(4-tert-butylbenzene-sulfonylamino)-3-(3-methoxyphenyl)-1H-indole-2-carboxylic acid ethyl ester (250 mg, 0.49 mmol) with 9.1 ml of a 1 M NaOH solution in ethanol/water (1/1), 190 mg (81%) of the product is obtained.

NMR (300 MHz, DMSO-d6): δ 1.24 (s, 9H), 3.78 (s, 3H), 6.84 (d, 1H), 6.90-6.92 (m, 2H), 7.07-7.12 (m, 2H), 7.28-7.36 (m, 2H), 7.51 (d, 2H), 7.57 (d, 2H), 9.85 (s, 1H), 11.79 (s, 1H), 12.90 (br, 1H).

5-(4-tert-Butylbenzenesulonylamino)-3-(3-methoxyphenyl)-1H-indole-2-carboxylic acid-(2-dimethylaminoethyl)amide: According to Instructions 1, after the reaction of 5-(4-tert-butylbenzenesulfonylamino)-3-(3-methoxyphenyl)-1H-indole-2-carboxylic acid (190 mg, 0.40 mmol) with N,N-dimethylethylenediamine (0.044 ml, 0.40 mmol) and chromatographic purification (silica gel, dichloromethane/methanol (0-20% methanol)), 120 mg (55%) of the product is obtained.

NMR (300 MHz, DMSO-d6): δ 1.24 (s, 9H), 2.02 (s, 6H), 2.23 (t, 2H), 3.23 (q, 2H), 3.79 (s, 3H), 6.83-6.97 (m, 2H), 6.96-7.09 (m, 4H), 7.31-7.40 (m, 2H), 7.50 (d, 2H), 7.56 (d, 2H), 9.80 (br, 1H), 11.71 (s, 1H).

### Example 44

### 5-(4-tert-Butylbenzenesulfonylamino)-3-(3-trifluoromethylphenyl)-1H-indole-2-carboxylic acid-(2-dimethylaminoethyl)amide

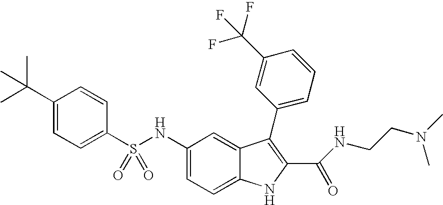 5-(4-tert-Butylbenzenesulfonylamino)-3-(3-trifluoromethylphenyl)-1H-indole-2-carboxylic acid ethyl ester: According to Instructions 6, after the reaction of 3-bromo-5-(4-tert-butylbenzenesulfonylamino)-1H-indole-2-carboxylic acid ethyl ester (400 mg, 0.83 mmol) with 3-trifluoromethylphenylboronic acid (226 mg, 1.19 mmol) and chromatographic purification (silica gel, hexane/ethyl acetate (0-100% ethyl acetate)), 300 mg (66%) of the product is obtained.

NMR (300 MHz, DMSO-d6): δ 1.16 (t, 3H), 1.22 (s, 9H), 4.21 (q, 2H), 7.09 (s, 1H), 7.15 (dd, 1H), 7.41 (d, 1H), 7.49 (d, 2H), 7.56-7.59 (m, 3H), 7.65-7.75 (m, 3H), 9.95 (s, 1H), 12.08 (s, 1H).

5-(4-tert-Butylbenzenesulonylamino)-3-(3-trifluoromethylphenyl)-1H-indole-2-carboxylic acid: According to Instructions 5, after the reaction of 5-(4-tert-butylbenzenesulfonylamino)-3-(3-trifluoromethylphenyl) 1H-indole-2-carboxylic acid ethyl ester (300 mg, 0.55 mmol) with 10.2 ml of a 1 M NaOH solution in ethanol/water (1/1), 270 mg (95%) of the product is obtained.

NMR (300 MHz, DMSO-d6): δ 1.24 (s, 9H), 7.08-7.18 (m, 2H), 7.40 (d, 1H), 7.52 (d, 2H), 7.58-7.75 (m, 6H), 9.91 (s, 1H), 11.99 (s, 1H), 12.40 (br, 1H).

5-(4-tert-Butylbenzenesulonylamino)-3-(3-trifluoromethylphenyl)-1H-indole-2-carboxylic acid-(2-dimethylaminoethyl)amide: According to Instructions 1, after the reaction of 5-(4-tert-butylbenzenesulfonylamino)-3-(3-trifluoromethylphenyl)-1H-indole-2-carboxylic acid (270 mg, 0.52 mmol) with N,N-dimethylethylenediamine (0.057 ml, 0.52 mmol) and chromatographic purification (silica gel, dichloromethane/methanol (0-20% methanol)), 180 mg (59%) of the product is obtained.

NMR (300 MHz, DMSO-d6): δ 1.22 (s, 9H), 2.05 (s, 6H), 2.25 (t, 2H), 3.24 (q, 2H), 7.05-7.09 (m, 2H), 7.35 (d, 1H), 7.44-7.51 (m, 3H), 7.57-7.61 (m, 4H), 7.66-7.75 (m, 2H), 9.89 (s, 1H), 11.86 (s, 1H).

### Example 45

### 5-(4-tert-Butylbenzenesulfonylamino)-3-(3-fluorophenyl)-1H-indole-2-carboxylic acid-(2-hydroxyethyl)amide

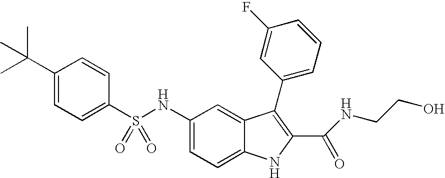

According to Instructions 1, after the reaction of 5-(4-tert-butylbenzene-sulfonylamino)-3-(3-fluorophenyl)-1H-indole-2-carboxylic acid (150 mg, 0.32 mmol) with ethanolamine (0.019 ml, 0.32 mmol) and chromatographic purification (silica gel, dichloromethane/methanol (0-20% methanol)), 27 mg (16%) of the product is obtained.

NMR (300 MHz, DMSO-d6): δ 1.24 (s, 9H), 3.24 (q, 2H), 3.40 (q, 2H), 4.64 (t, 1H), 7.05-7.21 (m, 5H), 7.34 (d, 1H), 7.42-7.61 (m, 6H), 9.83 (s, 1H), 11.79 (s, 1H).

### Example 46

### 5-(4-tert-Butylbenzenesulfonylamino)-3-(3-fluorophenyl)-1H-indole-2-carboxylic acid-(tetrahydropyran-4-yl)amide

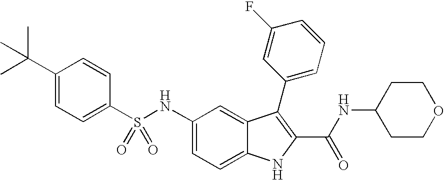

According to Instructions 1, after the reaction of 5-(4-tert-butylbenzene-sulfonylamino)-3-(3-fluorophenyl)-1H-indole-2-carboxylic acid (150 mg, 0.32 mmol) with 4-aminotetrahydropyran (33 mg, 0.32 mmol) and chromatographic purification (silica gel, dichloromethane/methanol (0-20% methanol)), 57 mg (33%) of the product is obtained.

NMR (300 MHz, DMSO-d6): δ 1.24 (s, 9H), 1.32-1.36 (m, 2H), 1.67-1.71 (m, 2H), 3.33-3.38 (m, 2H+H₂O), 3.74-3.78 (m, 2H), 3.87-3.94 (m, 1H), 7.04-7.20 (m, 5H), 7.34 (d, 1H), 7.43-7.73 (m, 6H), 9.84 (s, 1H), 11.81 (s, 1H).

### Example 47

### 5-(4-tert-Butylbenzenesulfonylamino)-3-(3-fluorophenyl)-1H-indole-2-carboxylic acid-(2-acetylaminoethyl)amide

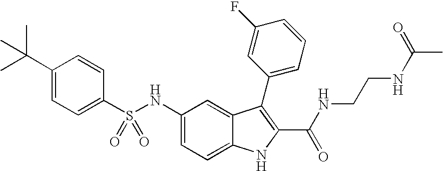

According to Instructions 1, after the reaction of 5-(4-tert-butylbenzene-sulfonylamino)-3-(3-fluorophenyl)-1H-indole-2-carboxylic acid (150 mg, 0.32 mmol) with N-acetylethylenediamine (0.03 ml, 0.32 mmol) and chromatographic purification (silica gel, dichloromethane/methanol (0-20% methanol)), 45 mg (26%) of the product is obtained.

NMR (300 MHz, DMSO-d6): δ 1.24 (s, 9H), 1.77 (s, 3H), 3.10 (q, 2H), 3.20 (q, 2H), 7.08-7.20 (m, 5H), 7.35 (d, 1H), 7.43-7.59 (m, 5H), 7.76 (t, 1H), 7.84 (t, 1H); 9.83 (s, 1H), 11.76 (s, 1H).

### Example 48

### 5-(4-tert-Butylbenzenesulfonylamino)-3-(3-fluorophenyl)-1H-indole-2-carboxylic acid-(2-morpholin-4-ylethyl)amide

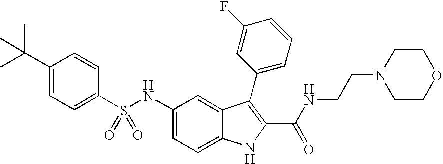

According to Instructions 1, after the reaction of 5-(4-tert-butylbenzene-sulfonylamino)-3-(3-fluorophenyl)-1H-indole-2-carboxylic acid (150 mg, 0.32 mmol) with 4-(2-aminoethyl)morpholine (0.042 ml, 0.32 mmol) and chromatographic purification (silica gel, dichloromethane/methanol (0-20% methanol)), 58 mg (32%) of the product is obtained.

NMR (300 MHz, DMSO-d6): δ 1.24 (s, 9H), 2.26-2.33 (m, 6H), 3.27-3.33 (m, 2H+H₂O), 3.44-3.46 (m, 4H), 7.05-7.15 (m, 4H), 7.19-7.26 (m, 2H), 7.34 (d, 1H), 7.47-7.62 (m, 5H), 9.82 (s, 1H), 11.82 (s, 1H).

### Example 49

### 5-(4-tert-Butylbenzenesulfonylamino)-3-(3-methoxyphenyl)-1H-indole-2-carboxylic acid-(2-hydroxyethyl)amide

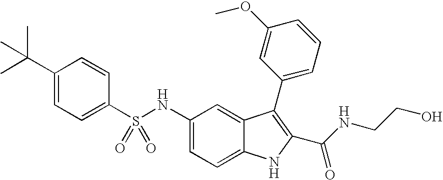

According to Instructions 1, after the reaction of 5-(4-tert-butylbenzene-sulfonylamino)-3-(3-methoxyphenyl)-1H-indole-2-carboxylic acid (200 mg, 0.42 mmol) with ethanolamine (0.025 ml, 0.42 mmol) and chromatographic purification (silica gel, dichloromethane/methanol (0-20% methanol)), 48 mg (22%) of the product is obtained.

NMR (300 MHz, DMSO-d6): δ 1.24 (s, 9H), 3.21-3.31 (m, 2H), 3.36-3.42 (m, 2H), 3.79 (s, 3H), 4.62 (t, 1H), 6.85-6.95 (m, 3H), 7.04 (dd, 1H), 7.14 (d, 1H), 7.29-7.38 (m, 3H), 7.50 (d, 2H), 7.57 (d, 2H), 9.82 (s, 1H), 11.70 (s, 1H).

### Example 50

### 5-(4-tert-Butylbenzenesulfonylamino)-3-(3-methoxyphenyl)-1H-indole-2-carboxylic acid-(2-acetylaminoethyl)amide

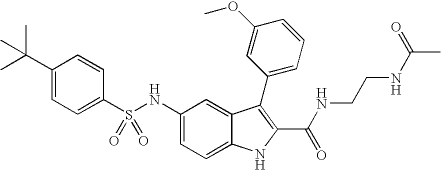

According to Instructions 1, after the reaction of 5-(4-tert-butylbenzene-sulfonylamino)-3-(3-methoxyphenyl)-1H-indole-2-carboxylic acid (200 mg, 0.42 mmol) with N-acetylethylenamine (0.04 ml, 0.42 mmol) and chromatographic purification (silica gel, dichloromethane/methanol (0-20% methanol)), 110 mg (47%) of the product is obtained.

NMR (300 MHz, DMSO-d6): δ 1.24 (s, 9H), 1.76 (s, 3H), 3.07-3.11 (m, 2H), 3.18-3.24 (m, 2H), 3.79 (s, 3H), 6.85-6.95 (m, 3H), 7.04 (dd, 1H), 7.17 (s, 1H), 7.31-7.37 (m, 2H), 7.50-7.62 (m, 5H), 7.81 (t, 1H), 9.83 (s, 1H), 11.68 (s, 1H).

### Example 51

### 5-(4-tert-Butylbenzenesulfonylamino)-3-pyridin-3-yl-1H-indole-2-carboxylic acid-(2-acetylaminoethyl)amide

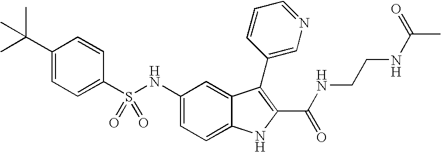

According to Instructions 1, after the reaction of 5-(4-tert-butylbenzene-sulfonylamino)-3-pyridin-3-yl-1H-indole-2-carboxylic acid (210 mg, 0.47 mmol) with N-acetylethylenediamine (0.045 ml, 0.47 mmol) and chromatographic purification (silica gel, dichloromethane/methanol (0-20% methanol)), 66 mg (26%) of the product is obtained.

NMR (300 MHz, DMSO-d6): δ 1.25 (s, 9H), 1.78 (s, 3H), 3.09-3.14 (m, 2H), 3.16-3.24 (m, 2H), 7.06-7.09 (m, 2H), 7.37 (d, 1H), 7.44 (dd, 1H), 7.52 (d, 2H), 7.57 (d, 2H), 7.67-7.69 (m, 1H), 7.86 (t, 1H), 7.93 (t, 1H), 8.47 (d, 1H), 8.53 (dd, 1H), 9.83 (s, 1H), 11.85 (s, 1H).

### Example 52

### 5-(4-tert-Butylbenzenesulfonylamino)-3-pyridin-3-yl-1H-indole-2-carboxylic acid-(tetrahydropyran-4yl)amide

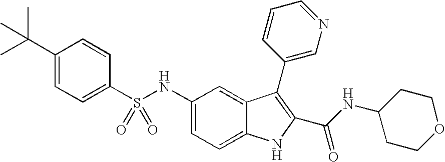

According to Instructions 1, after the reaction of 5-(4-tert-butylbenzene-sulfonylamino)-3-pyridin-3-yl-1H-indole-2-carboxylic acid (140 mg, 0.31 mmol) with 4-aminotetrahydropyran (32 mg, 0.31 mmol) and chromatographic purification (silica gel, dichloromethane/methanol (0-50% methanol)), 30 mg (18%) of the product is obtained.

NMR (300 MHz, DMSO-d6): δ 1.24 (s, 9H), 1.34-1.39 (m, 2H), 1.67-1.71 (m, 2H), 3.32-3.38 (m, 2H+H₂O),3.76-3.80 (m, 2H), 3.87-3.97 (m, 1H), 7.07 (dd, 1H), 7.11 (s, 1H), 7.37 (d, 1H), 7.45 (dd, 1H), 7.52 (d, 2H), 7.58 (d, 2H), 7.66-7.69 (m, 1H), 7.82 (d, 1H), 8.46 (d, 1H), 8.51 (d, 1H), 9.83 (s, 1H), 11.87 (s, 1H).

### Example 53

### 5-(4-tert-Butylbenzenesulfonylamino)-3-pyridin-4-yl-1H-indole-2-carboxylic acid-(2-acetylaminoethyl)amide

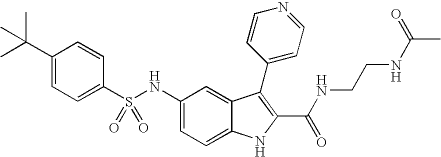 5-(4-tert-Butylbenzenesulonylamino)-3-pyridin-4-yl-1H-indole-2-carboxylic acid ethyl ester:

According to Instructions 6, after the reaction of 3-bromo-5-(4-tert-butylbenzene-sulfonylamino)-1H-indole-2-carboxylic acid ethyl ester (500 mg, 1.05 mmol) with pyridine-4-boronoic acid (184 mg, 1.5 mmol) and chromatographic purification (silica gel, hexane/ethyl acetate (0-100% ethyl acetate)), 310 mg (62%) of the product is obtained.

NMR (300 MHz, DMSO-d6): δ 1.18 (t, 3H), 1.24 (s, 9H), 4.23 (q, 2H), 7.11-7.16 (m, 2H), 7.32 (d, 2H), 7.42 (d, 1H), 7.53 (d, 2H), 7.59 (d, 2H), 8.62 (d, 2H), 9.95 (s, 1H), 12.17 (s, 1H).
5-(4-tert-Butylbenzenesulonylamino)-3-pyridin-4-yl-1H-indole-2-carboxylic acid:

According to Instructions 5, after the reaction of 5-(4-tert-butylbenzenesulfonyl-amino)-3-pyridin-4-yl-1H-indole-2-carboxylic acid ethyl ester (310 mg, 0.65 mmol) with 12 ml of a 1 M NaOH solution in ethanol/water (1/1), 290 mg (99%) of the product is obtained.

NMR (300 MHz, DMSO-d6): δ 1.24 (s, 9H), 7.10 (dd, 1H), 7.20 (d, 1H), 7.41 (d, 1H), 7.52-7.63 (m, 6H), 8.73 (d, 2H), 9.99 (s, 1H), 12.24 (s, 1H).

5-(4-tert-Butylbenzenesulfonylamino)-3-pyridin-4-yl-1H-indole-2-carboxylic acid-(2-acetyl-aminoethyl)amide: According to Instructions 1, after the reaction of 5-(4-tert-butylbenzenesulfonylamino)-3-pyridin-4-yl-1H-indole-2-carboxylic acid (180 mg, 0.4 mmol) with N-acetylethylenediamine (0.038 ml, 0.4 mmol) and chromatographic purification (silica gel, dichloromethane/methanol (0-20% methanol)), 22 mg (10%) of the product is obtained.

NMR (300 MHz, DMSO-d6): δ 1.24 (s, 9H), 1.77 (s, 3H), 3.12-3.16 (m, 2H), 3.20-3.27 (m, 2H), 7.04 (dd, 1H), 7.19 (d, 1H), 7.27 (d, 2H), 7.36 (d, 1H), 7.54 (d, 2H), 7.59 (d, 2H), 7.89 (t, 1H), 8.09 (t, 1H), 8.58 (d, 2H), 9.95 (br, 1H), 11.97 (br, 1H).

### Example 54

### 5-(4-tert-Butylbenzenesulfonylamino)-3-pyridin-4-yl-1H-indole-2-carboxylic acid-(2-morpholin-4-ylethyl)amide

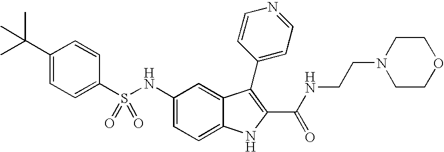

According to Instructions 1, after the reaction of 5-(4-tert-butylbenzene-sulfonylamino)-3-pyridin-4-yl-1H-indole-2-carboxylic acid (145 mg, 0.32 mmol) with 4-(2-aminoethyl)morpholine (0.042 ml, 0.32 mmol) and chromatographic purification (silica gel, dichloromethane/methanol (0-20% methanol)), 58 mg (32%) of the product is obtained.

NMR (300 MHz, DMSO-d6): δ 1.24 (s, 9H), 2.30-2.38 (m, 6H), 3.30-3.34 (m, 2H+H₂O), 3.45-3.47 (m, 4H), 7.05 (dd, 1H), 7.16 (d, 1H), 7.30 (d, 2H), 7.35 (d, 1H), 7.49-7.65 (m, 5H), 8.61 (d, 2H), 9.89 (br, 1H), 11.97 (br, 1H).

### Example 55

### 5-(4-tert-Butylbenzenesulfonylamino)-3-pyridin-4-yl-1H-indole-2-carboxylic acid-(tetrahydropyran-4-yl)amide

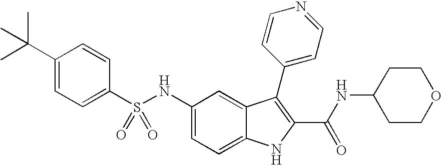

According to Instructions 1, after the reaction of 5-(4-tert-butylbenzene-sulfonylamino)-3-pyridin-4-yl-1H-indole-2-carboxylic acid (145 mg, 0.32 mmol) with 4-aminotetrahydropyran (33 mg, 0.32 mmol) and chromatographic purification (silica gel, dichloromethane/methanol (0-20% methanol)), 90 mg (53%) of the product is obtained.

NMR (300 MHz, DMSO-d6): δ 1.24 (s, 9H), 1.31-1.49 (m, 2H), 1.69-1.74 (m, 2H), 3.39-3.50 (m, 2H), 3.79-3.82 (m, 2H), 3.89-4.00 (m, 1H), 7.06 (dd, 1H), 7.24 (d, 1H), 7.27 (d, 2H), 7.37 (d, 1H), 7.54 (d, 2H), 7.60 (d, 2H), 8.03 (d, 1H), 8.57 (d, 2H), 9.87 (s, 1H), 11.96 (s, 1H).

### Example 56

### 5-(4-tert-Butylbenzenesulfonylamino)-3-(3-methylphenyl)-1H-indole-2-carboxylic acid-(2-acetylaminoethyl)amide

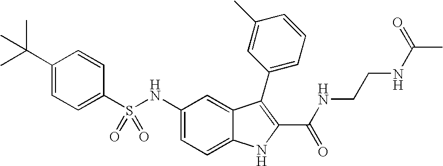

5-(4-tert-Butylbenzenesulfonylamino)-3-(3-methylphenyl)-1H-indole-2-carboxylic acid ethyl ester: According to Instructions 6, after the reaction of 3-bromo-5-(4-tert-butylbenzenesulfonylamino)-1H-indole-2-carboxylic acid ethyl ester (500 mg, 1.05 mmol) with m-toluoylboronic acid (204 mg, 1.5 mmol) and chromatographic purification (silica gel, hexane/ethyl acetate (0-100% ethyl acetate)), 330 mg (67%) of the product is obtained.

NMR (300 MHz, DMSO-d6): δ 1.18 (t, 3H), 1.24 (s, 9H), 2.55 (s, 3H), 4.19 (q, 2H), 7.06-7.21 (m, 5H), 7.30 (t, 1H), 7.37 (d, 1H), 7.52 (d, 2H), 7.58 (d, 2H), 9.88 (s, 1H), 11.88 (s, 1H).

5-(4-tert-Butylbenzenesulonylamino)-3-(3-methylphenyl)-1H-indole-2-carboxylic acid: According to Instructions 5, after the reaction of 5-(4-tert-butylbenzene-sulfonylamino)-3-(3-methylphenyl)-1H-indole-2-carboxylic acid ethyl ester (330 mg, 0.67 mmol) with 12 ml of a 1 M NaOH solution in ethanol/water (1/1), 300 mg (97%) of the product is obtained.

NMR (300 MHz, DMSO-d6): δ 1.24 (s, 9H), 2.35 (s, 3H), 7.05-7.15 (m, 5H), 7.29 (t, 1H), 7.34 (d, 1H), 7.51 (d, 2H), 7.57 (d, 2H), 9.85 (s, 1H), 11.77 (s, 1H).

5-(4-tert-Butylbenzenesulfonylamino)-3-(3-methylphenyl)-1H-indole-2-carboxylic acid-(2-acetylaminoethyl)amide: According to Instructions 1, after the reaction of 5-(4-tert-butylbenzenesulfonylamino)-3-(3-methylphenyl)-1H-indole-2-carboxylic acid (210 mg, 0.45 mmol) with N-acetylethylenediamine (0.043 ml, 0.45 mmol) and chromatographic purification (silica gel, dichloromethane/methanol (0-20% methanol)), 120 mg (49%) of the product is obtained.

NMR (300 MHz, DMSO-d6): δ 1.24 (s, 9H), 1.75 (s, 3H), 2.36 (s, 3H), 3.06-3.10 (m, 2H), 3.19-3.23 (m, 2H), 7.02-7.09 (m, 2H), 7.13-7.23 (m, 3H), 7.33-7.35 (m, 2H), 7.41 (t, 1H), 7.52 (d, 2H), 7.57 (d, 2H), 7.81 (t, 1H), 9.82 (s, 1H), 11.65 (s, 1H).

### Example 57

### 5-(4-tert-Butylbenzenesulfonylamino)-3-(3-methylphenyl)-1H-indole-2-carboxylic acid-(2-hydroxyethyl)amide

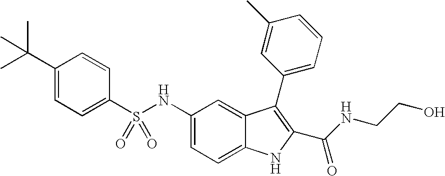

According to Instructions 1, after the reaction of 5-(4-tert-butylbenzene-sulfonylamino)-3-(3-methylphenyl)-1H-indole-2-carboxylic acid (210 mg, 0.45 mmol) with ethanolamine (0.027 ml, 0.45 mmol) and chromatographic purification (silica gel, dichloromethane/methanol (0-20% methanol)), 22 mg (10%) of the product is obtained.

NMR (300 MHz, DMSO-d6): δ 1.24 (s, 9H), 2.36 (s, 3H), 3.16-3.26 (m, 2H), 3.35-3.39 (m, 2H), 4.62 (t, 1H), 7.02-7.09 (m, 3H), 7.17-7.25 (m, 3H), 7.31-7.36 (m, 2H), 7.52 (d, 2H), 7.58 (d, 2H), 9.81 (s, 1H), 11.68 (s, 1H).

### Example 58

### 5-(4-tert-Butylbenzenesulfonylamino)-3-(3-methylphenyl)-1H-indole-2-carboxylic acid-(2-morpholin-4-ylethyl)amide

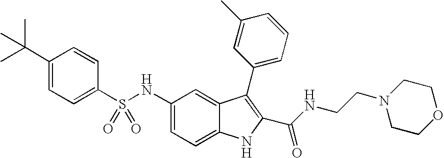

According to Instructions 1, after the reaction of 5-(4-tert-butylbenzene-sulfonylamino)-3-(3-methylphenyl)-1H-indole-2-carboxylic acid (150 mg, 0.32 mmol) with 4-(2-aminoethyl)morpholine (0.042 ml, 0.32 mmol) and chromatographic purification (silica gel, dichloromethane/methanol (0-20% methanol)), 90 mg (49%) of the product is obtained.

NMR (300 MHz, DMSO-d6): δ 1.24 (s, 9H), 2.13-2.19 (m, 4H), 2.27 (t, 2H), 2.37 (s, 3H), 3.27-3.39 (m, 2H), 3.39 (br, 4H), 6.92 (t, 1H), 7.02-7.10 (m, 3H), 7.15 (s, 1H), 7.22 (d, 1H), 7.34-7.40 (m, 2H), 7.52 (d, 2H), 7.57 (d, 2H), 9.80 (s, 1H), 11.70 (s, 1H).

### Example 59

### 5-(4-tert-Butylbenzenesulfonylamino)-3-(3-methylphenyl)-1H-indole-2-carboxylic acid-(tetrahydropyran-4-yl)amide

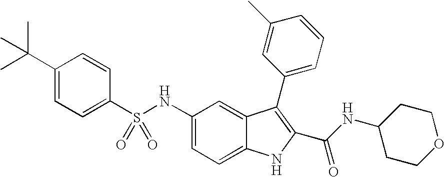

According to Instructions 1, after the reaction of 5-(4-tert-butylbenzene-sulfonylamino)-3-(3-methylphenyl)-1H-indole-2-carboxylic acid (150 mg, 0.32 mmol) with 4-aminotetrahydropyran (33 mg, 0.32 mmol) and purification by means of HPLC, 50 mg (29%) of the product is obtained.

NMR (300 MHz, DMSO-d6): δ 1.20-1.35 (m, 11H), 1.63-1.71 (m, 2H), 2.36 (s, 3H), 3.33-3.38 (m, 2H+H₂O), 3.68-3.72 (m, 2H), 3.87-3.99 (m, 1H), 7.02 (dd, 1H), 7.08-7.11 (m, 2H), 7.17-7.20 (m, 3H), 7.30-7.37 (m, 2H), 7.51 (d, 2H), 7.57 (d, 2H), 9.83 (s, 1H), 11.70 (s, 1H).

### Example 60

### 5-(4-tert-Butylbenzenesulfonylamino)-3-pyridin-3-yl-1H-indole-2-carboxylic acid-(2-morpholin-4-ylethyl)amide

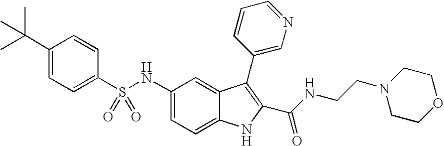

According to Instructions 1, after the reaction of 5-(4-tert-butylbenzene-sulfonylamino)-3-pyridin-3-yl-1H-indole-2-carboxylic acid (140 mg, 0.31 mmol) with 4-(2-aminoethyl)morpholine (0.04 ml, 0.31 mmol) and chromatographic purification (silica gel, dichloromethane/methanol (0-50% methanol)), 30 mg (17%) of the product is obtained.

NMR (300 MHz, DMSO-d6): δ 1.24 (s, 9H), 2.31-2.40 (m, 6H), 3.48 (br, 4H), 3.59-3.60 (m, 2H), 7.05-7.11 (m, 2H), 7.37 (d, 1H), 7.46-7.59 (m, 6H), 7.70 (d, 1H), 8.50 (d, 1H), 8.55 (dd, 1H), 9.83 (s, 1H), 11.86 (s, 1H).

### Example 61

### 5-(4-tert-Butylbenzenesulfonylamino)-3-(3-methoxyphenyl)-1H-indole-2-carboxylic acid-(tetrahydropyran-4-yl)amide

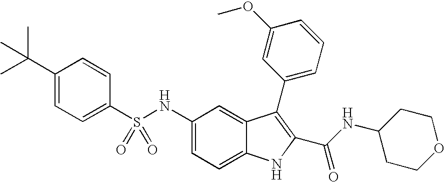

According to Instructions 1, after the reaction of 5-(4-tert-butylbenzene-sulfonylamino)-3-(3-methoxyphenyl)-1H-indole-2-carboxylic acid (125 mg, 0.26 mmol) with 4-aminotetrahydropyran (29 mg, 0.29 mmol) and chromatographic purification (silica gel, dichloromethane/methanol (0-20% methanol)), 110 mg (75%) of the product is obtained.

NMR (300 MHz, DMSO-d6): δ 1.24-1.36 (m, 11H), 1.67-1.70 (m, 2H), 3.31-3.38 (m, 2H+H₂O), 3.69-3.79 (m, 2H), 3.79 (s, 3H), 3.86-3.98 (m, 1H), 6.86-6.96 (m, 3H), 7.04 (dd, 1H), 7.16 (d, 1H), 7.31-7.38 (m, 3H), 7.51 (d, 2H), 7.57 (d, 2H), 9.83 (s, 1H), 11.73 (s, 1H).

### Example 62

### 5-(4-tert-Butylbenzenesulfonylamino)-3-(3-methoxyphenyl)-1H-indole-2-carboxylic acid-(2-morpholin-4-ylethyl)amide

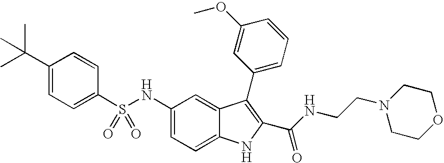

According to Instructions 1, after the reaction of 5-(4-tert-butylbenzene-sulfonylamino)-3-(3-methoxyphenyl)-1H-indole-2-carboxylic acid (125 mg, 0.26 mmol) with 4-(2-aminoethyl)morpholine (0.034 ml, 0.26 mmol) and chromatographic purification (silica gel, dichloromethane/methanol (0-20% methanol)), 60 mg (39%) of the product is obtained.

NMR (300 MHz, DMSO-d6): δ 1.24 (s, 9H), 2.21 (br, 4H), 2.28 (t, 2H), 3.26-3.30 (m, 2H+H₂O), 3.40 (br, 4H), 3.79 (s, 3H), 6.86-6.90 (m, 2H), 6.97-7.08 (m, 4H), 7.31-7.42 (m, 2H), 7.51 (d, 2H), 7.56 (d, 2H), 9.81 (d, 1H), 11.72 (s, 1H).

The following compounds were synthesized according to the production methods of the disclosed examples.

| Exam- | | |
|---|---|---|
| ple | Structure | compound name |
| 63 | 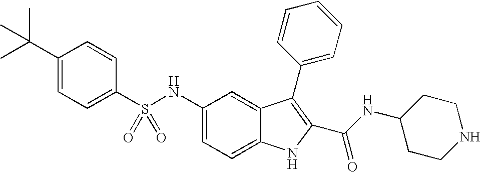 | 5-(4-tert-Butylbenzenesulfonylamino)-3-phenyl- 1H-indole-2-carboxylic acid piperidin-4-ylamide |
| 64 | 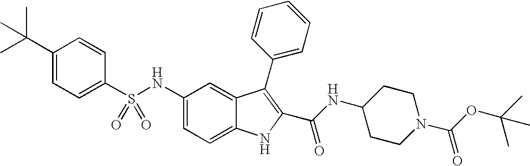 | 4-{[5-(4-tert-Butylbenzenesulfonylamino)-3- phenyl-1H-indole-2-carbonyl]-piperidine-1- carboxylic acid-tert-butyl ester |
| 65 | 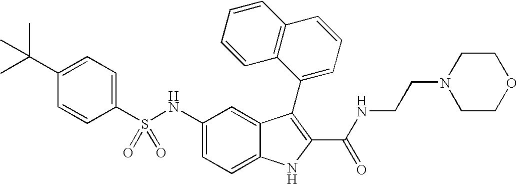 | 5-(4-tert-Butyl-benzenesulfonyl-amino)-3- naphthalen-1-yl-1H-indole-2-carboxylic acid (2- morpholin-4-yl-ethyl)-amide |
| 66 | 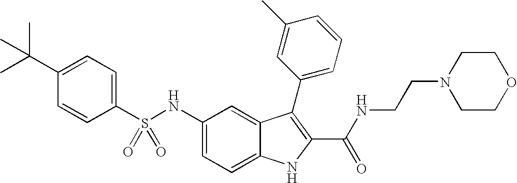 | 5-(4-tert-Butyl-benzenesulfonyl-amino)-3-m-tolyl- 1H-indole-2-carboxylic acid (2-morpholin-4-yl- ethyl)-amide |
| 67 | 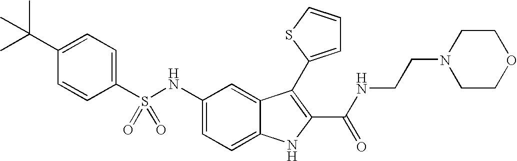 | 5-(4-tert-Butyl-benzenesulfonyl-amino)-3-thiophen- 2-yl-1H-indole-2-carboxylic acid (2-morpholin-4-yl- ethyl)-amide |
| 68 | 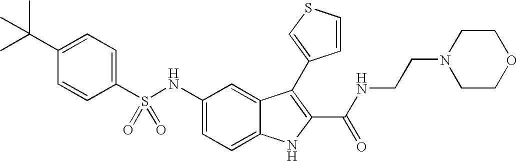 | 5-(4-tert-Butyl-benzenesulfonyl-amino)-3-thiophen- 3-yl-1H-indole-2-carboxylic acid (2-morpholin-4-yl- ethyl)-amide |
| 69 | 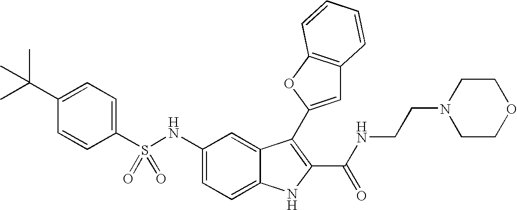 | 3-Benzofuran-2-yl-5-(4-tert-butyl- benzenesulfonylamino)-1H-indole-2-carboxylic acid (2-morpholin-4-yl-ethyl)-amide |
| 70 | 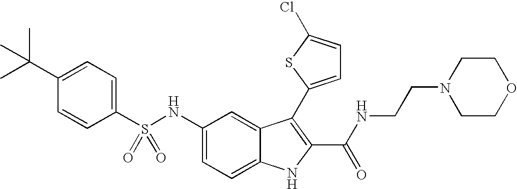 | 5-(4-tert-Butyl-benzenesulfonyl-amino)-3-(5- chloro-thiophen-2-yl)-1H-indole-2-carboxylic acid (2-morpholin-4-yl-ethyl)-amide |
| 71 | 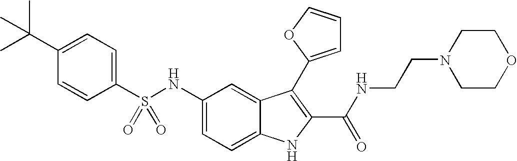 | 5-(4-tert-Butyl-benzenesulfonyl-amino)-3-furan-2- yl-1H-indole-2-carboxylic acid (2-morpholin-4-yl- ethyl)-amide |
| 72 | 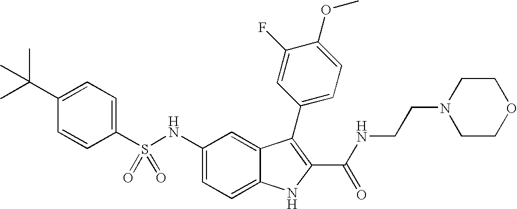 | 5-(4-tert-Butyl-benzenesulfonyl-amino)-3-(3-fluoro- 4-methoxy-phenyl)-1H-indole-2-carboxylic acid (2- morpholin-4-yl-ethyl)-amide |
| 73 | 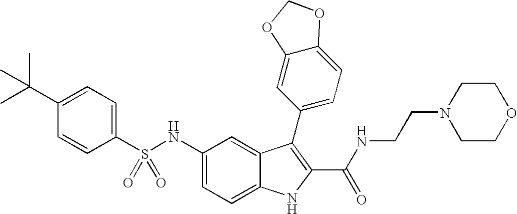 | 3-Benzo[1,3]dioxol-5-yl-5-(4-tert-butyl- benzenesulfonylamino)-1H-indole-2-carboxylic acid (2morpholin-4-yl-ethyl)-amide |
| 74 | 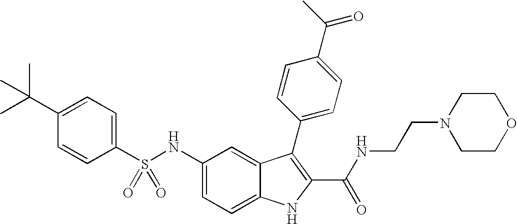 | 3-(4-Acetyl-phenyl)-5-(4-tert-butyl- benzenesulfonylamino)-1H-indole-2-carboxylic acid (2-morpholin-4-yl-ethyl)-amide |
| 75 | 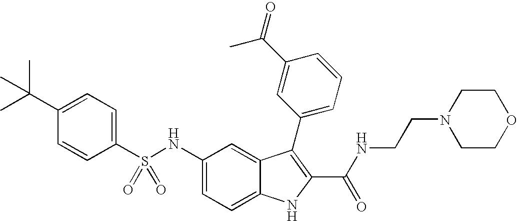 | 3-(3-Acetyl-phenyl)-5-(4-tert-butyl- benzenesulfonylamino)-1H-indole-2-carboxylic acid (2-morpholin-4-yl-ethyl)-amide |
| 76 | 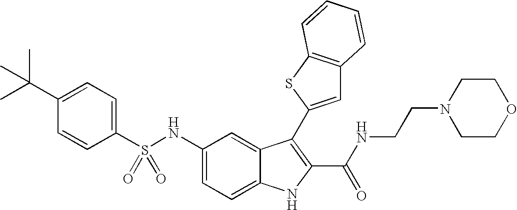 | 3-Benzo[b]thiophen-2-yl-5-(4-tert-butyl- benzenesulfonylamino)-1H-indole-2-carboxylic acid (2-morpholin-4-yl-ethyl)-amide |
| 77 | 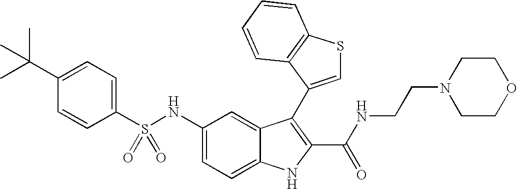 | 3-Benzo[b]thiophen-3-yl-5-(4-tert-butyl- benzenesulfonylamino)-1H-indole-2-carboxylic acid (2-morpholin-4-yl-ethyl)-amide |
| 78 | 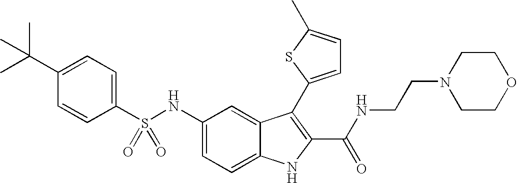 | 5-(4-tert-Butyl-benzenesulfonyl-amino)-3-(5- methyl-thiophen-2-yl)-1H-indole-2-carboxylic acid (2-morpholin-4-yl-ethyl)-amide |
| 79 | 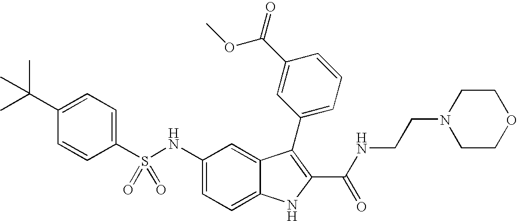 | 3-[5-(4-tert-Butyl-benzenesulfonyl-amino)-2-(2- morpholin-4-yl-ethylcarbamoyl)-1H-indol-3-yl]- benzoic acid-methyl ester |
| 80 | 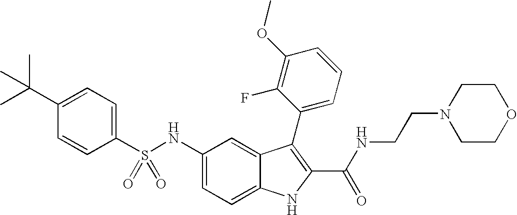 | 5-(4-tert-Butyl-benzenesulfonyl-amino)-3-(2-fluoro- 3-methoxy-phenyl)-1H-indole-2-carboxylic acid (2- morpholin-4-yl-ethyl)-amide |
| 81 | 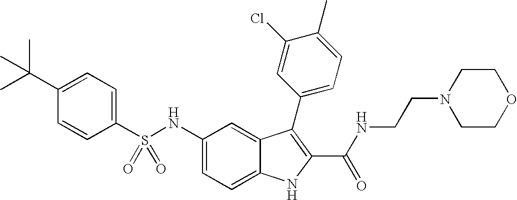 | 5-(4-tert-Butyl-benzenesulfonyl-amino)-3-(3- chloro-4-methyl-phenyl)-1H-indole-2-carboxylic acid (2-morpholin-4-yl-ethyl)-amide |
| 82 | 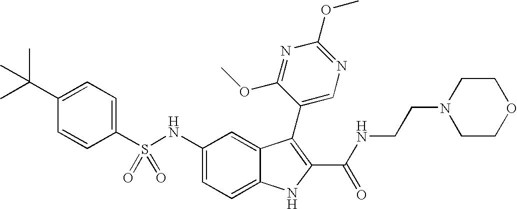 | 5-(4-tert-Butyl-benzenesulfonyl-amino)-3-(2,4- dimethoxy-pyrimidin-5-yl)-1H-indole-2-carboxylic acid (2-morpholin-4-yl-ethyl)-amide |
| 83 | 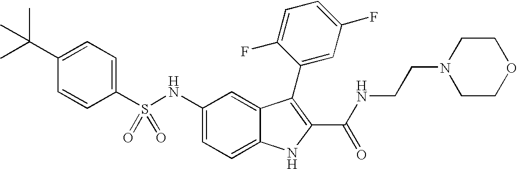 | 5-(4-tert-Butyl-benzenesulfonyl-amino)-3-(2,5- difluoro-phenyl)-1H-indole-2-carboxylic acid (2- morpholin-4-yl-ethyl)-amide |
| 84 | 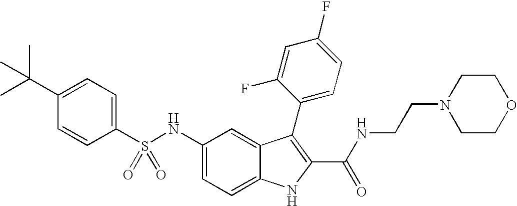 | 5-(4-tert-Butyl-benzenesulfonyl-amino)-3-(2,4- difluoro-phenyl)-1H-indole-2-carboxylic acid (2- morpholin-4-yl-ethyl)-amide |
| 85 | 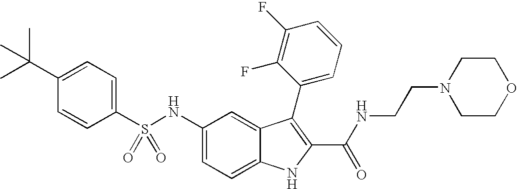 | 5-(4-tert-Butyl-benzenesulfonyl-amino)-3-(2,3- difluoro-phenyl)-1H-indole-2-carboxylic acid (2- morpholin-4-yl-ethyl)-amide |
| 86 | 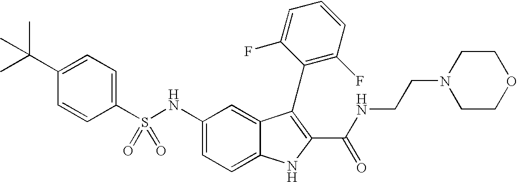 | 5-(4-tert-Butyl-benzenesulfonyl-amino)-3-(2,6- difluoro-phenyl)-1H-indole-2-carboxylic acid (2- morpholin-4-yl-ethyl)-amide |
| 87 | 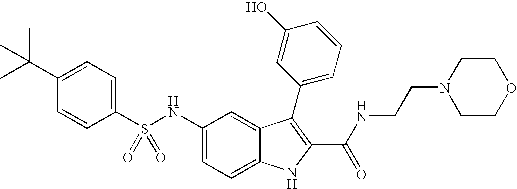 | 5-(4-tert-Butyl-benzenesulfonyl-amino)-3-(3- hydroxy-phenyl)-1H-indole-2-carboxylic acid (2- morpholin-4-yl-ethyl)-amide |
| 88 | 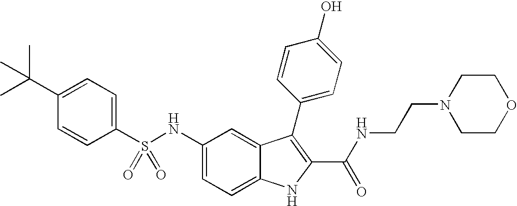 | 5-(4-tert-Butyl-benzenesulfonyl-amino)-3-(4- hydroxy-phenyl)-1H-indole-2-carboxylic acid (2- morpholin-4-yl-ethyl)-amide |
| 89 | 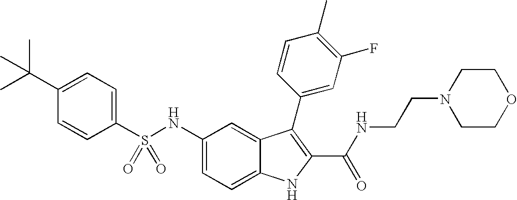 | 5-(4-tert-Butyl-benzenesulfonyl-amino)-3-(3-fluoro- 4-methyl-phenyl)-1H-indole-2-carboxylic acid (2- morpholin-4-yl-ethyl)-amide |
| 90 | 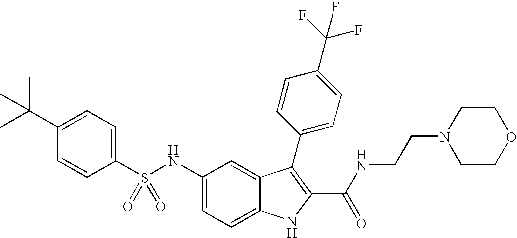 | 5-(4-tert-Butyl-benzenesulfonyl-amino)-3-(4- trifluoromethyl-phenyl)-1H-indole-2-carboxylic acid (2-morpholin-4-yl-ethyl)-amide |
| 91 | 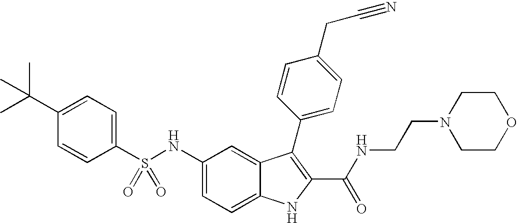 | 5-(4-tert-Butyl-benzenesulfonyl-amino)-3-(4- cyanomethyl-phenyl)-1H-indole-2-carboxylic acid (2-morpholin-4-yl-ethyl)-amide |
| 92 | 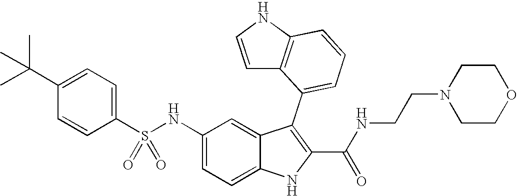 | 5-(4-tert-Butyl-benzenesulfonyl-amino)-1H,1′H- [3,4′]biindolyl-2-carboxylic acid (2-morpholin-4-yl- ethyl)-amide |
| 93 | 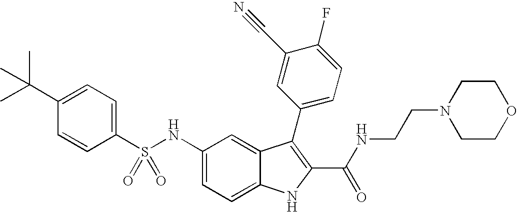 | 5-(4-tert-Butyl-benzenesulfonyl-amino)-3-(3- cyano-4-fluoro-phenyl)-1H-indole-2-carboxylic acid (2-morpholin-4-yl-ethyl)-amide |
| 94 | 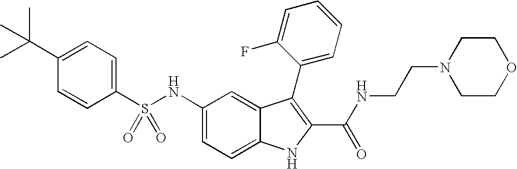 | 5-(4-tert-Butyl-benzenesulfonyl-amino)-3-(2-fluoro- phenyl)-1H-indole-2-carboxylic acid (2-morpholin- 4-yl-ethyl)-amide |
| 95 | 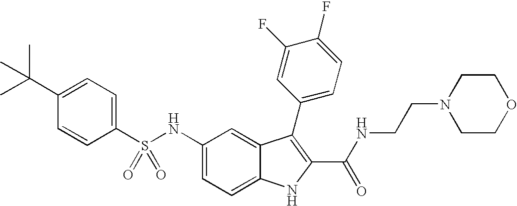 | 5-(4-tert-Butyl-benzenesulfonyl-amino)-3-(3,4- difluoro-phenyl)-1H-indole-2-carboxylic acid (2- morpholin-4-yl-ethyl)-amide |
| 96 | 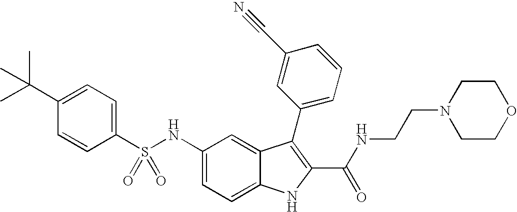 | 5-(4-tert-Butyl-benzenesulfonyl-amino)-3-(3- cyano-phenyl)-1H-indole-2-carboxylic acid (2- morpholin-4-yl-ethyl)-amide |
| 97 | 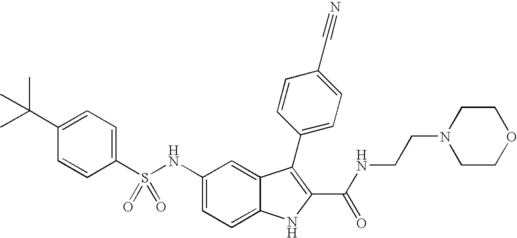 | 5-(4-tert-Butyl-benzenesulfonyl-amino)-3-(4- cyano-phenyl)-1H-indole-2-carboxylic acid (2- morpholin-4-yl-ethyl)-amide |
| 98 | 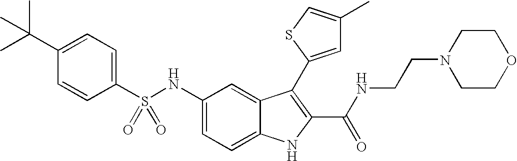 | 5-(4-tert-Butyl-benzenesulfonyl-amino)-3-(4- methyl-thiophen-2-yl)-1H-indole-2-carboxylic acid (2-morpholin-4-yl-ethyl)-amide |
| 99 | 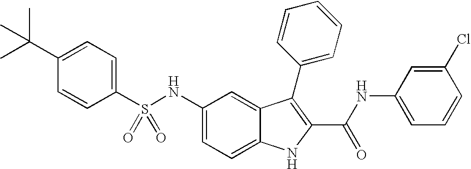 | 5-(4-tert-Butyl-benzenesulfonyl-amino)-3-phenyl- 1H-indole-2-carboxylic acid (3-chloro-phenyl)- amide |
| 100 | 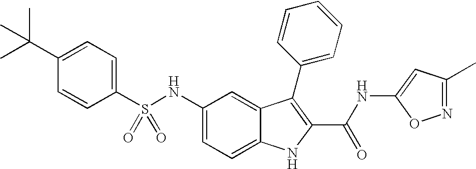 | 5-(4-tert-Butyl-benzenesulfonyl-amino)-3-phenyl- 1H-indole-2-carboxylic acid (3-methyl-isoxazol-5- yl)-amide |
| 101 | 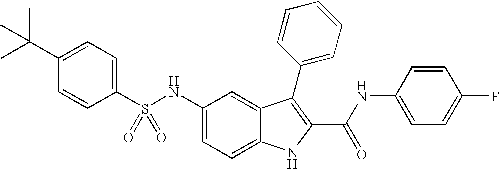 | 5-(4-tert-Butyl-benzenesulfonyl-amino)-3-phenyl- 1H-indole-2-carboxylic acid (4-fluoro-phenyl)- amide |
| 102 | 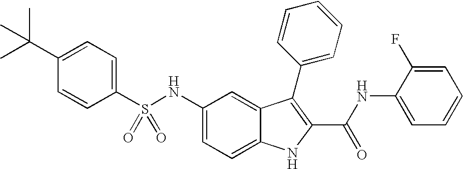 | 5-(4-tert-Butyl-benzenesulfonyl-amino)-3-phenyl- 1H-indole-2-carboxylic acid (2-fluoro-phenyl)- amide |
| 103 | 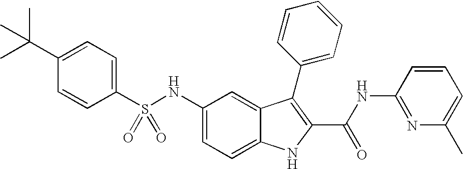 | 5-(4-tert-Butyl-benzenesulfonyl-amino)-3-phenyl- 1H-indole-2-carboxylic acid (6-methyl-pyridin-2- yl)-amide |
| 104 | 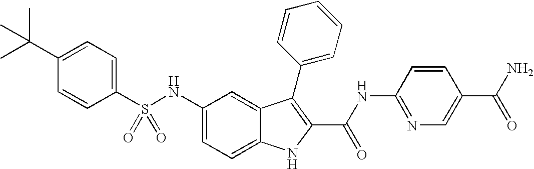 | 5-(4-tert-Butyl-benzenesulfonyl-amino)-3-phenyl- 1H-indole-2-carboxylic acid (5-carbamoyl-pyridin- 2-yl)-amide |
| 105 | 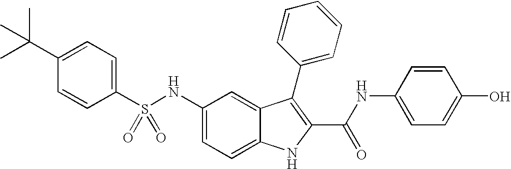 | 5-(4-tert-Butyl-benzenesulfonyl-amino)-3-phenyl- 1H-indole-2-carboxylic acid (4-hydroxy-phenyl)- amide |
| 106 | 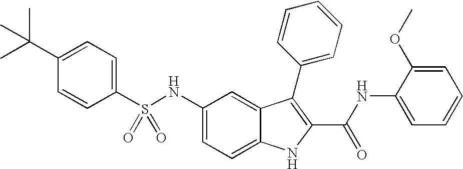 | 5-(4-tert-Butyl-benzenesulfonyl-amino)-3-phenyl- 1H-indole-2-carboxylic acid (2-methoxy-phenyl)- amide |
| 107 | 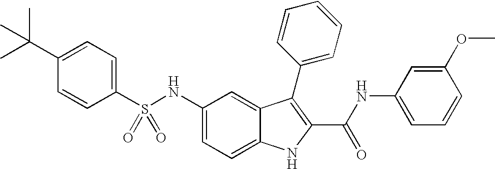 | 5-(4-tert-Butyl-benzenesulfonyl-amino)-3-phenyl- 1H-indole-2-carboxylic acid (3-methoxy-phenyl)- amide |
| 108 | 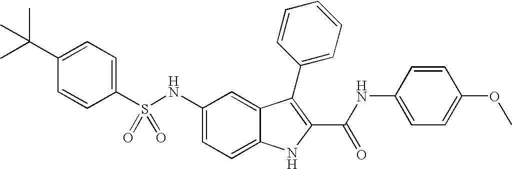 | 5-(4-tert-Butyl-benzenesulfonyl-amino)-3-phenyl- 1H-indole-2-carboxylic acid (4-methoxy-phenyl)- amide |
| 109 | 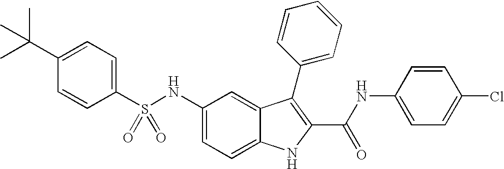 | 5-(4-tert-Butyl-benzenesulfonyl-amino)-3-phenyl- 1H-indole-2-carboxylic acid (4-chloro-phenyl)- amide |
| 110 | 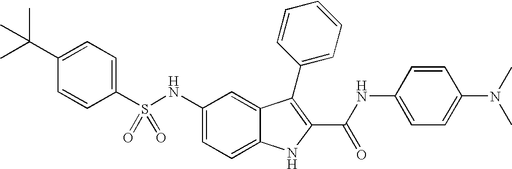 | 5-(4-tert-Butyl-benzenesulfonyl-amino)-3-phenyl- 1H-indole-2-carboxylic acid (4-dimethylamino- phenyl)-amide |
| 111 | 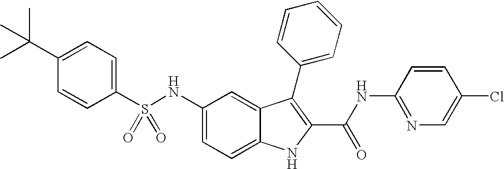 | 5-(4-tert-Butyl-benzenesulfonyl-amino)-3-phenyl- 1H-indole-2-carboxylic acid (5-chloro-pyridin-2-yl)- amide |
| 112 | 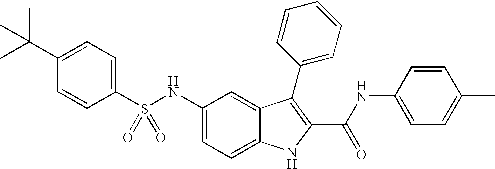 | 5-(4-tert-Butyl-benzenesulfonyl-amino)-3-phenyl- 1H-indole-2-carboxylic acid p-tolylamide |
| 113 | 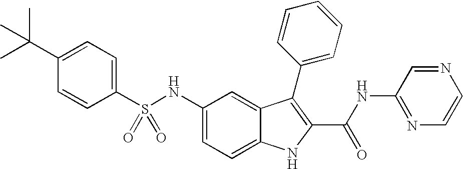 | 5-(4-tert-Butyl-benzenesulfonyl-amino)-3-phenyl- 1H-indole-2-carboxylic acid pyrazin-2-ylamide |
| 114 | 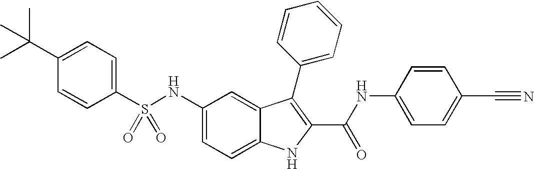 | 5-(4-tert-Butyl-benzenesulfonyl-amino)-3-phenyl- 1H-indole-2-carboxylic acid (4-cyano-phenyl)- amide |
| 115 | 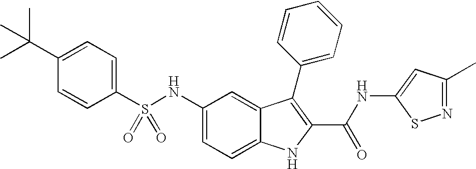 | 5-(4-tert-Butyl-benzenesulfonyl-amino)-3-phenyl- 1H-indole-2-carboxylic acid (3-methyl-isothiazol-5- yl)-amide |
| 116 | 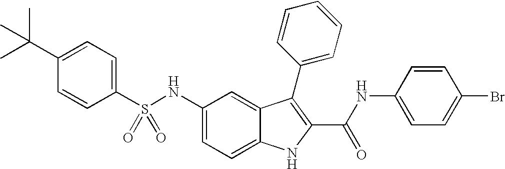 | 5-(4-tert-Butyl-benzenesulfonyl-amino)-3-phenyl- 1H-indole-2-carboxylic acid (4-bromo-phenyl)- amide |
| 117 | 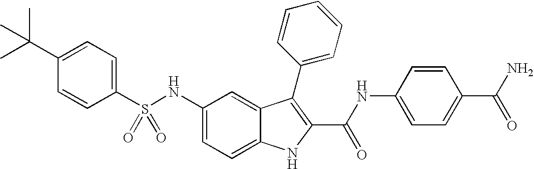 | 5-(4-tert-Butyl-benzenesulfonyl-amino)-3-phenyl- 1H-indole-2-carboxylic acid (4-carbamoyl-phenyl)- amide |
| 118 | 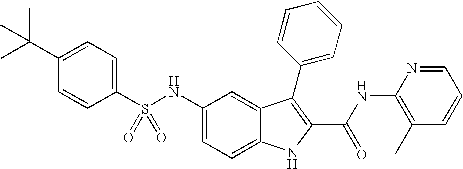 | 5-(4-tert-Butyl-benzenesulfonyl-amino)-3-phenyl- 1H-indole-2-carboxylic acid (3-methyl-pyridin-2- yl)-amide |
| 119 | 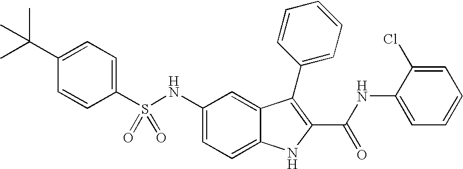 | 5-(4-tert-Butyl-benzenesulfonyl-amino)-3-phenyl- 1H-indole-2-carboxylic acid (2-chloro-phenyl)- amide |
| 120 | 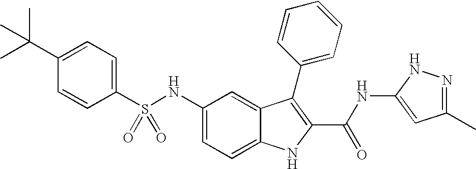 | 5-(4-tert-Butyl-benzenesulfonyl-amino)-3-phenyl- 1H-indole-2-carboxylic acid (5-methyl-2H-pyrazol- 3-yl)-amide |
| 121 | 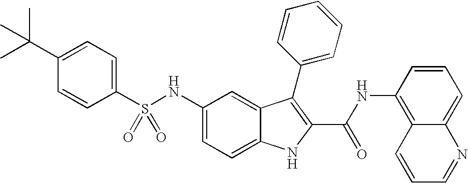 | 5-(4-tert-Butyl-benzenesulfonyl-amino)-3-phenyl- 1H-indole-2-carboxylic acid quinolin-5-ylamide |
| 122 | 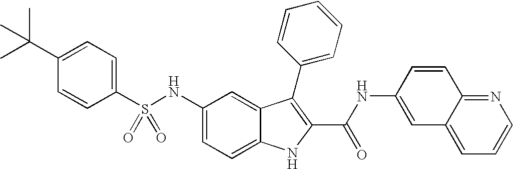 | 5-(4-tert-Butyl-benzenesulfonyl-amino)-3-phenyl- 1H-indole-2-carboxylic acid quinolin-6-ylamide |
| 123 | 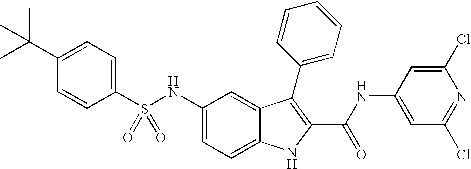 | 5-(4-tert-Butyl-benzenesulfonyl-amino)-3-phenyl- 1H-indole-2-carboxylic acid (2,6-dichloro-pyridin-4- yl)-amide |
| 124 | 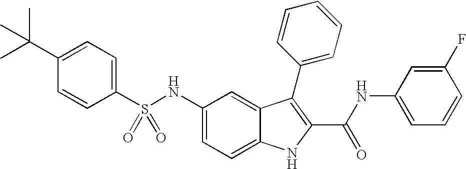 | 5-(4-tert-Butyl-benzenesulfonyl-amino)-3-phenyl- 1H-indole-2-carboxylic acid (3-fluoro-phenyl)- amide |
| 125 | 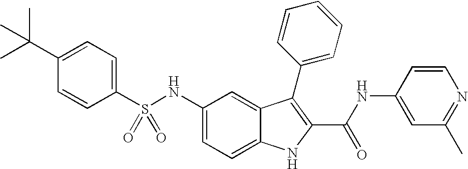 | 5-(4-tert-Butyl-benzenesulfonyl-amino)-3-phenyl- 1H-indole-2-carboxylic acid (2-methyl-pyridin-4- yl)-amide |
| 126 | 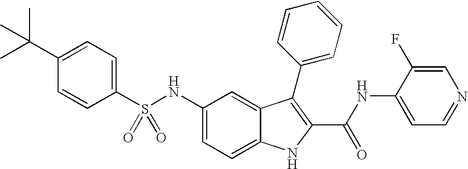 | 5-(4-tert-Butyl-benzenesulfonyl-amino)-3-phenyl- 1H-indole-2-carboxylic acid (3-fluoro-pyridin-4-yl)- amide |
| 127 | 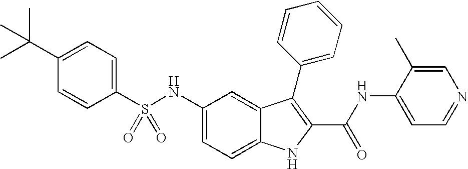 | 5-(4-tert-Butyl-benzenesulfonyl-amino)-3-phenyl- 1H-indole-2-carboxylic acid (3-methyl-pyridin-4- yl)-amide |
| 128 | 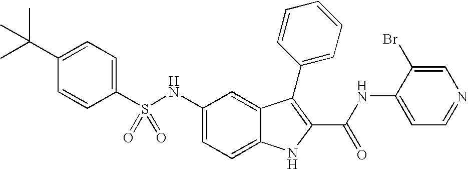 | 5-(4-tert-Butyl-benzenesulfonyl-amino)-3-phenyl- 1H-indole-2-carboxylic acid (3-bromo-pyridin-4-yl)- amide |
| 129 | 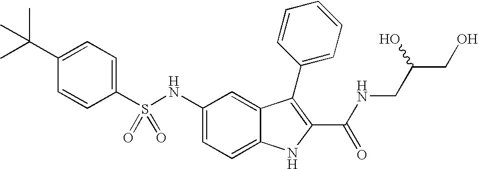 | 5-(4-tert-Butyl-benzenesulfonyl-amino)-3-phenyl- 1H-indole-2-carboxylic acid (2,3-dihydroxy-propyl)- amide |
| 130 | 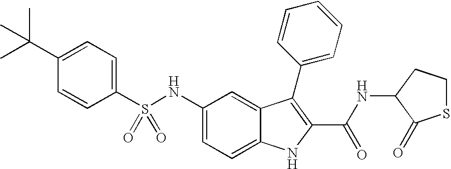 | 5-(4-tert-Butyl-benzenesulfonyl-amino)-3-phenyl- 1H-indole-2-carboxylic acid (2-oxo-tetrahydro- thiophen-3-yl)-amide |
| 131 | 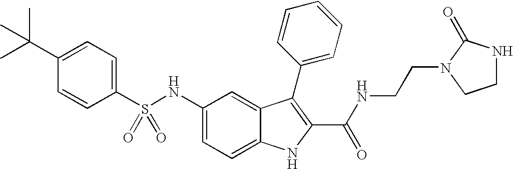 | 5-(4-tert-Butyl-benzenesulfonyl-amino)-3-phenyl- 1H-indole-2-carboxylic acid [2-(2-oxo-imidazolidin- 1-yl)-ethyl]-amide |
| 132 | 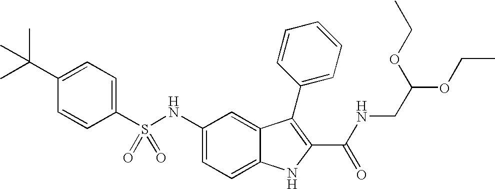 | 5-(4-tert-Butyl-benzenesulfonyl-amino)-3-phenyl- 1H-indole-2-carboxylic acid (2,2-diethoxy-ethyl)- amide |
| 133 | 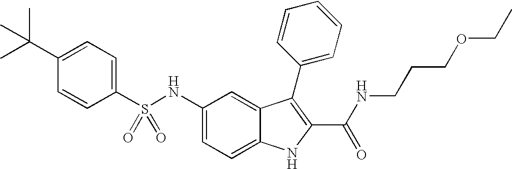 | 5-(4-tert-Butyl-benzenesulfonyl-amino)-3-phenyl- 1H-indole-2-carboxylic acid (3-ethoxy-propyl)- amide |
| 134 | 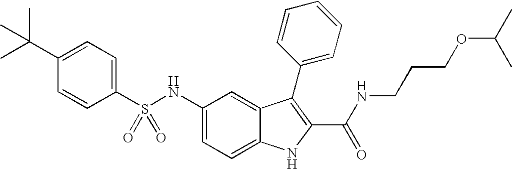 | 5-(4-tert-Butyl-benzenesulfonyl-amino)-3-phenyl- 1H-indole-2-carboxylic acid (3-isopropoxy-propyl)- amide |
| 135 | 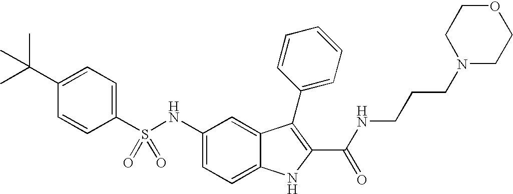 | 5-(4-tert-Butyl-benzenesulfonyl-amino)-3-phenyl- 1H-indole-2-carboxylic acid (3-morpholin-4-yl- propyl)-amide |
| 136 | 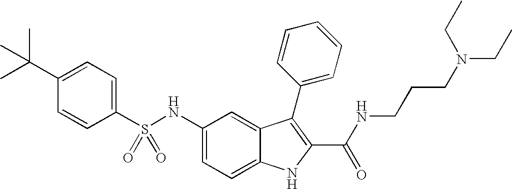 | 5-(4-tert-Butyl-benzenesulfonyl-amino)-3-phenyl- 1H-indole-2-carboxylic acid (3-diethylamino- propyl)-amide |
| 137 | 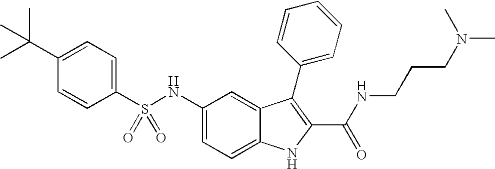 | 5-(4-tert-Butyl-benzenesulfonyl-amino)-3-phenyl- 1H-indole-2-carboxylic acid (3-dimethylamino- propyl)-amide |
| 138 | 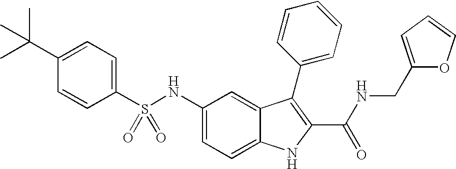 | 5-(4-tert-Butyl-benzenesulfonyl-amino)-3-phenyl- 1H-indole-2-carboxylic acid (furan-2-ylmethyl)- amide |
| 139 | 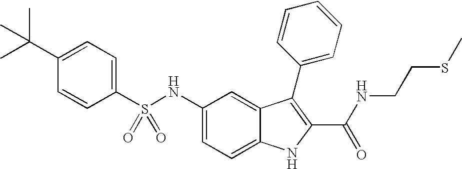 | 5-(4-tert-Butyl-benzenesulfonyl-amino)-3-phenyl- 1H-indole-2-carboxylic acid (2-methylsulfanyl- ethyl)-amide |
| 140 | 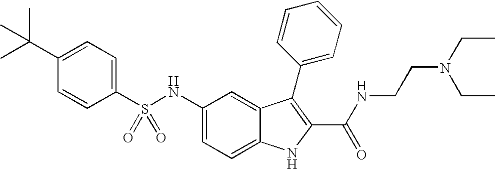 | 5-(4-tert-Butyl-benzenesulfonyl-amino)-3-phenyl- 1H-indole-2-carboxylic acid (2-diethylamino-ethyl)- amide |
| 141 | 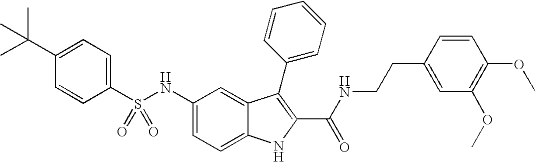 | 5-(4-tert-Butyl-benzenesulfonyl-amino)-3-phenyl- 1H-indole-2-carboxylic acid [2-(3,4-dimethoxy- phenyl)-ethyl]-amide |
| 142 | 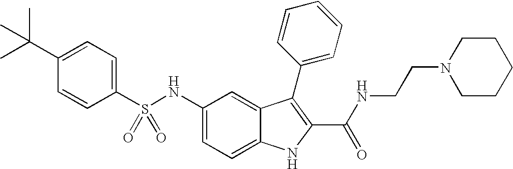 | 5-(4-tert-Butyl-benzenesulfonyl-amino)-3-phenyl- 1H-indole-2-carboxylic acid (2-piperidin-1-yl-ethyl)- amide |
| 143 | 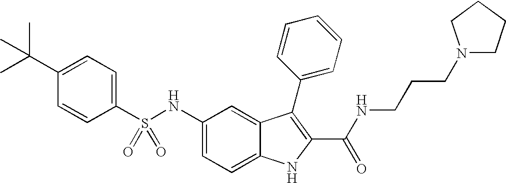 | 5-(4-tert-Butyl-benzenesulfonyl-amino)-3-phenyl- 1H-indole-2-carboxylic acid (3-pyrrolidin-1-yl- propyl)-amide |
| 144 | 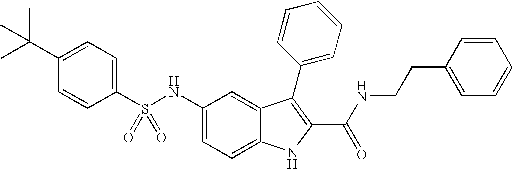 | 5-(4-tert-Butyl-benzenesulfonyl-amino)-3-phenyl- 1H-indole-2-carboxylic acid phenethyl-amide |
| 145 | 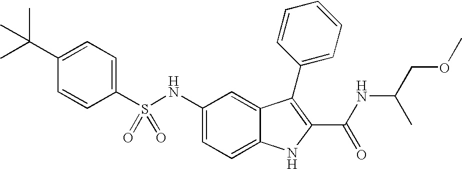 | 5-(4-tert-Butyl-benzenesulfonyl-amino)-3-phenyl- 1H-indole-2-carboxylic acid (2-methoxy-1-methyl- ethyl)-amide |
| 146 | 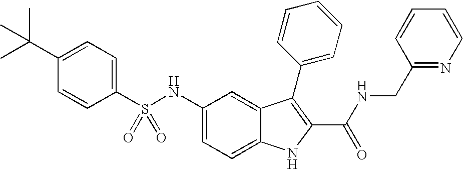 | 5-(4-tert-Butyl-benzenesulfonyl-amino)-3-phenyl- 1H-indole-2-carboxylic acid (pyridin-2-ylmethyl)- amide |
| 147 | 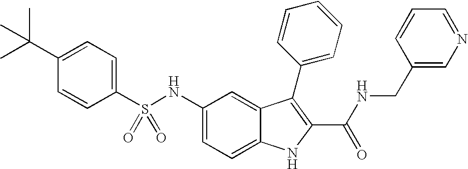 | 5-(4-tert-Butyl-benzenesulfonyl-amino)-3-phenyl- 1H-indole-2-carboxylic acid (pyridin-3-ylmethyl)- amide |
| 148 | 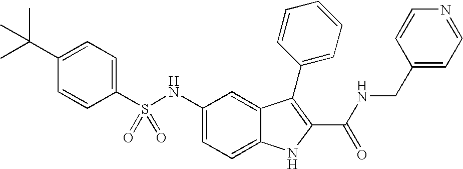 | 5-(4-tert-Butyl-benzenesulfonyl-amino)-3-phenyl- 1H-indole-2-carboxylic acid (pyridin-4-ylmethyl)- amide |
| 149 | 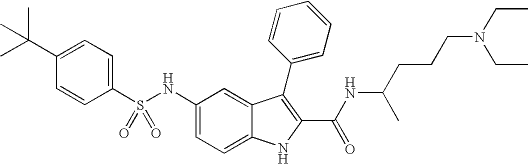 | 5-(4-tert-Butyl-benzenesulfonyl-amino)-3-phenyl- 1H-indole-2-carboxylic acid (4-diethylamino-1- methyl-butyl)-amide |
| 150 | 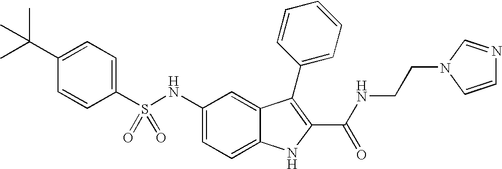 | 5-(4-tert-Butyl-benzenesulfonyl-amino)-3-phenyl- 1H-indole-2-carboxylic acid (2-imidazol-1-yl-ethyl)- amide |
| 151 | 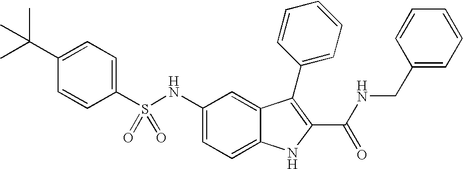 | 5-(4-tert-Butyl-benzenesulfonyl-amino)-3-phenyl- 1H-indole-2-carboxylic acid benzylamide |
| 152 | 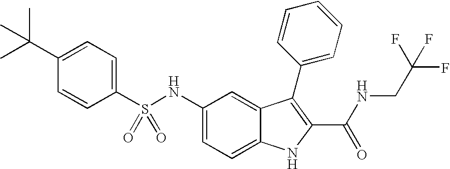 | 5-(4-tert-Butyl-benzenesulfonyl-amino)-3-phenyl- 1H-indole-2-carboxylic acid (2,2,2-trifluoro-ethyl)- amide |
| 153 | 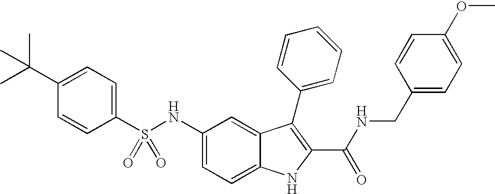 | 5-(4-tert-Butyl-benzenesulfonyl-amino)-3-phenyl- 1H-indole-2-carboxylic acid 4-methoxy- benzylamide |
| 154 | 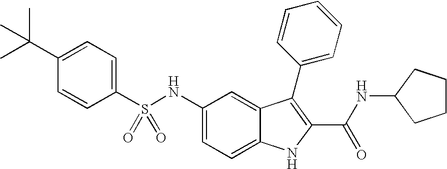 | 5-(4-tert-Butyl-benzenesulfonyl-amino)-3-phenyl- 1H-indole-2-carboxylic acid cyclopentylamide |
| 155 | 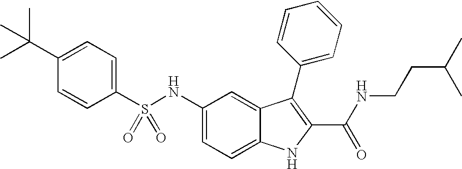 | 5-(4-tert-Butyl-benzenesulfonyl-amino)-3-phenyl- 1H-indole-2-carboxylic acid (3-methyl-butyl)-amide |
| 156 | 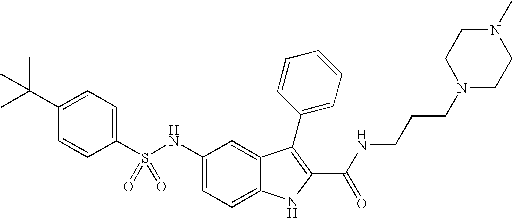 | 5-(4-tert-Butyl-benzenesulfonyl-amino)-3-phenyl- 1H-indole-2-carboxylic acid [3-(4-methyl-piperazin- 1-yl)-propyl]-amide |
| 157 | 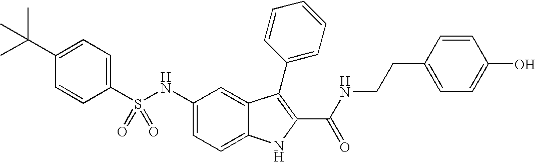 | 5-(4-tert-Butyl-benzenesulfonyl-amino)-3-phenyl- 1H-indole-2-carboxylic acid [2-(4-hydroxy-phenyl)- ethyl]-amide |
| 158 | 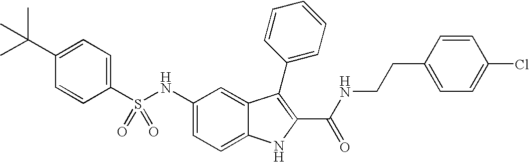 | 5-(4-tert-Butyl-benzenesulfonyl-amino)-3-phenyl- 1H-indole-2-carboxylic acid [2-(4-chloro-phenyl)- ethyl]-amide |
| 159 | 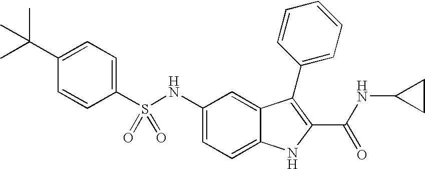 | 5-(4-tert-Butyl-benzenesulfonyl-amino)-3-phenyl- 1H-indole-2-carboxylic acid cyclopropylamide |
| 160 | 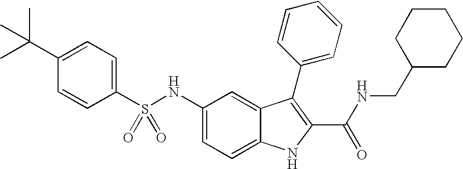 | 5-(4-tert-Butyl-benzenesulfonyl-amino)-3-phenyl- 1H-indole-2-carboxylic acid cyclohexylmethyl- amide |
| 161 | 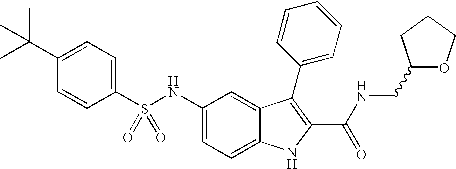 | 5-(4-tert-Butyl-benzenesulfonyl-amino)-3-phenyl- 1H-indole-2-carboxylic acid (tetrahydro-furan-2- ylmethyl)-amide |
| 162 | 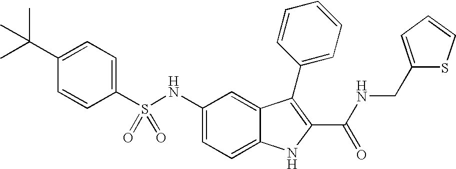 | 5-(4-tert-Butyl-benzenesulfonyl-amino)-3-phenyl- 1H-indole-2-carboxylic acid (thiophen-2-ylmethyl)- amide |
| 163 | 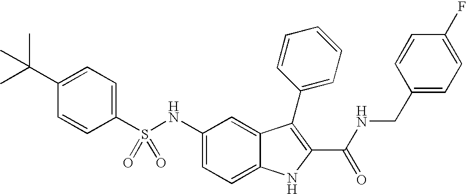 | 5-(4-tert-Butyl-benzenesulfonyl-amino)-3-phenyl- 1H-indole-2-carboxylic acid 4-fluoro-benzylamide |
| 164 | 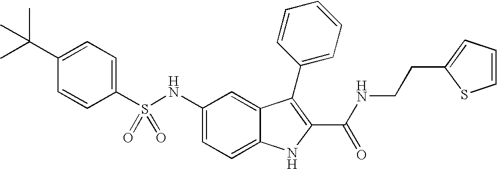 | 5-(4-tert-Butyl-benzenesulfonyl-amino)-3-phenyl- 1H-indole-2-carboxylic acid (2-thiophen-2-yl-ethyl)- amide |
| 165 | 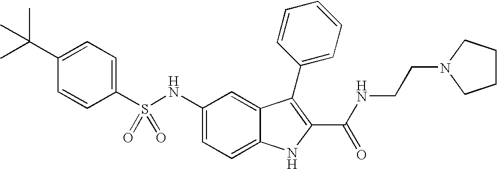 | 5-(4-tert-Butyl-benzenesulfonyl-amino)-3-phenyl- 1H-indole-2-carboxylic acid (2-pyrrolidin-1-yl- ethyl)-amide |
| 166 | 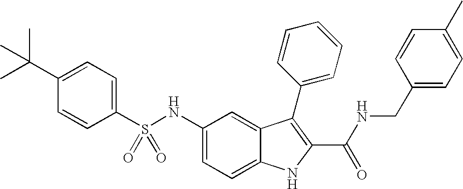 | 5-(4-tert-Butyl-benzenesulfonyl-amino)-3-phenyl- 1H-indole-2-carboxylic acid 4-methyl-benzylamide |
| 167 | 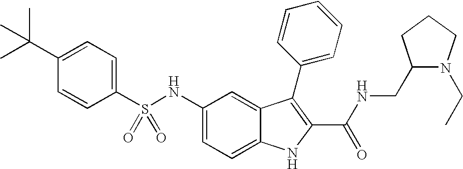 | 5-(4-tert-Butyl-benzenesulfonyl-amino)-3-phenyl- 1H-indole-2-carboxylic acid (1-ethyl-pyrrolidin-2- ylmethyl)-amide |
| 168 | 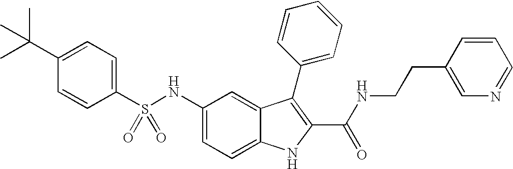 | 5-(4-tert-Butyl-benzenesulfonyl-amino)-3-phenyl- 1H-indole-2-carboxylic acid (2-pyridin-3-yl-ethyl)- amide |
| 169 | 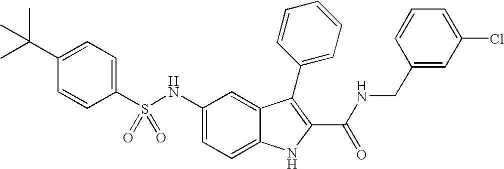 | 5-(4-tert-Butyl-benzenesulfonyl-amino)-3-phenyl- 1H-indole-2-carboxylic acid 3-chloro-benzylamide |
| 170 | 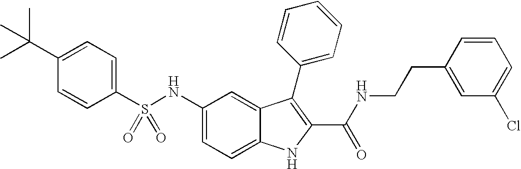 | 5-(4-tert-Butyl-benzenesulfonyl-amino)-3-phenyl- 1H-indole-2-carboxylic acid [2-(3-chloro-phenyl)- ethyl]-amide |
| 171 | 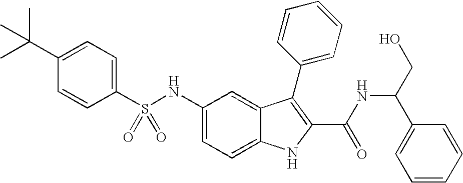 | 5-(4-tert-Butyl-benzenesulfonyl-amino)-3-phenyl- 1H-indole-2-carboxylic acid ((R)-2-hydroxy-1- phenyl-ethyl)-amide |
| 172 | 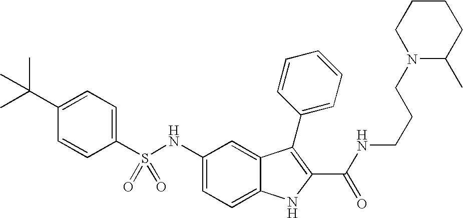 | 5-(4-tert-Butyl-benzenesulfonyl-amino)-3-phenyl- 1H-indole-2-carboxylic acid [3-(2-methyl-piperidin- 1-yl)-propyl]-amide |
| 173 | 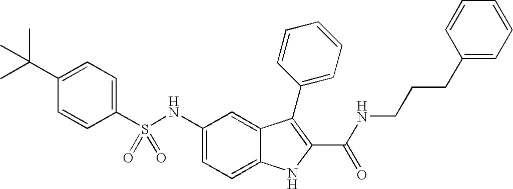 | 5-(4-tert-Butyl-benzenesulfonyl-amino)-3-phenyl- 1H-indole-2-carboxylic acid (3-phenyl-propyl)- amide |
| 174 | 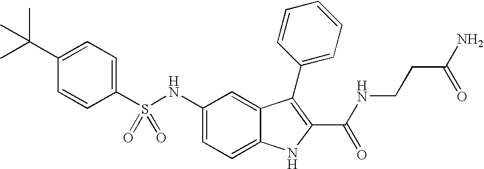 | 5-(4-tert-Butyl-benzenesulfonyl-amino)-3-phenyl- 1H-indole-2-carboxylic acid (2-carbamoyl-ethyl)- amide |
| 175 | 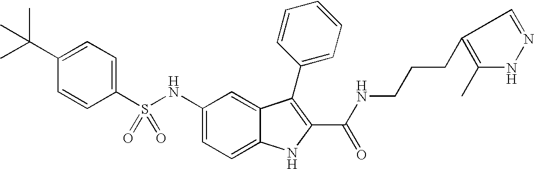 | 5-(4-tert-Butyl-benzenesulfonyl-amino)-3-phenyl- 1H-indole-2-carboxylic acid [3-(5-methyl-1H- pyrazol-4-yl)-propyl]-amide |
| 176 | 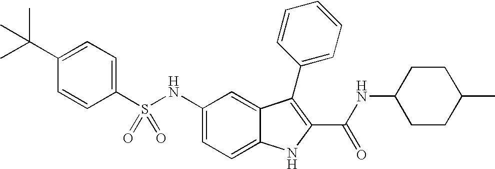 | 5-(4-tert-Butyl-benzenesulfonyl-amino)-3-phenyl- 1H-indole-2-carboxylic acid (4-methyl-cyclohexyl)- amide |
| 177 | 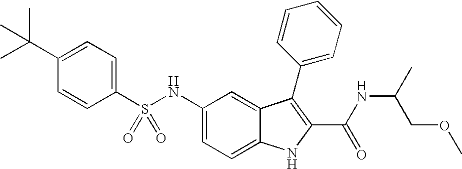 | 5-(4-tert-Butyl-benzenesulfonyl-amino)-3-phenyl- 1H-indole-2-carboxylic acid ((S)-2-methoxy-1- methyl-ethyl)-amide |
| 178 | 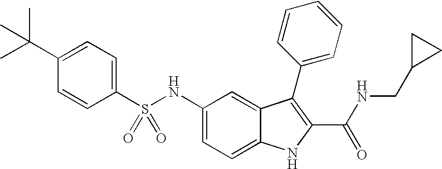 | 5-(4-tert-Butyl-benzenesulfonyl-amino)-3-phenyl- 1H-indole-2-carboxylic acid cyclopropylmethyl- amide |
| 179 | 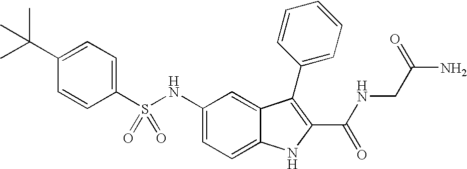 | 5-(4-tert-Butyl-benzenesulfonyl-amino)-3-phenyl- 1H-indole-2-carboxylic acid carbamoylmethyl- amide |
| 180 | 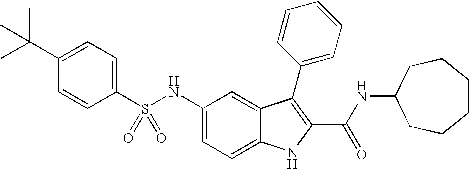 | 5-(4-tert-Butyl-benzenesulfonyl-amino)-3-phenyl- 1H-indole-2-carboxylic acid cycloheptylamide |
| 181 | 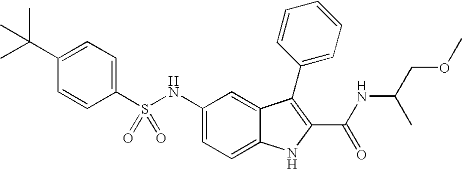 | 5-(4-tert-Butyl-benzenesulfonyl-amino)-3-phenyl- 1H-indole-2-carboxylic acid ((S)-2-methoxy-1- methyl-ethyl)-amide |
| 182 | 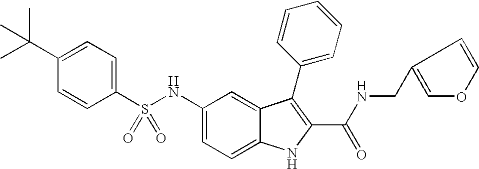 | 5-(4-tert-Butyl-benzenesulfonyl-amino)-3-phenyl- 1H-indole-2-carboxylic acid (furan-3-ylmethyl)- amide |
| 183 | 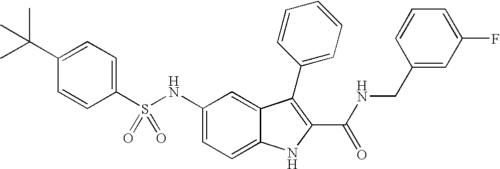 | 5-(4-tert-Butyl-benzenesulfonyl-amino)-3-phenyl- 1H-indole-2-carboxylic acid 3-fluoro-benzylamide |
| 184 | 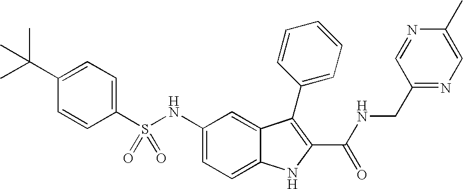 | 5-(4-tert-Butyl-benzenesulfonyl-amino)-3-phenyl- 1H-indole-2-carboxylic acid (5-methyl-pyrazin-2- ylmethyl)-amide |
| 185 | 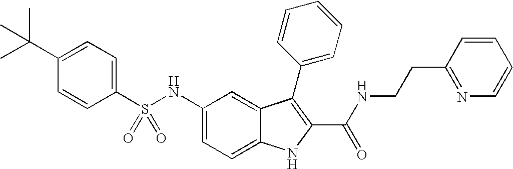 | 5-(4-tert-Butyl-benzenesulfonyl-amino)-3-phenyl- 1H-indole-2-carboxylic acid (2-pyridin-2-yl-ethyl)- amide |
| 186 | 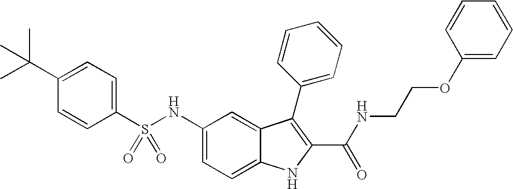 | 5-(4-tert-Butyl-benzenesulfonyl-amino)-3-phenyl- 1H-indole-2-carboxylic acid (2-phenoxy-ethyl)- amide |
| 187 | 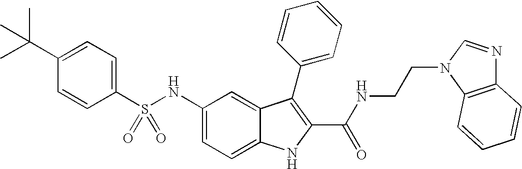 | 5-(4-tert-Butyl-benzenesulfonyl-amino)-3-phenyl- 1H-indole-2-carboxylic acid (2-benzoimidazol-1-yl- ethyl)-amide |
| 188 | 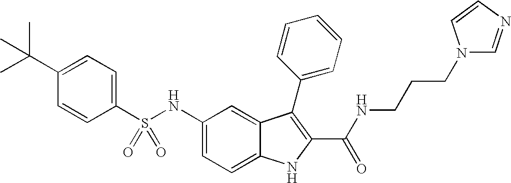 | 5-(4-tert-Butyl-benzenesulfonyl-amino)-3-phenyl- 1H-indole-2-carboxylic acid (3-imidazol-1-yl- propyl)-amide |
| 189 | 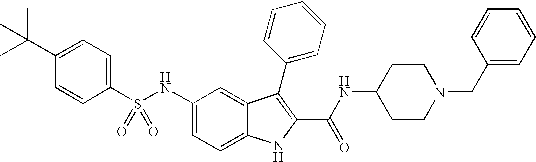 | 5-(4-tert-Butyl-benzenesulfonyl-amino)-3-phenyl- 1H-indole-2-carboxylic acid (1-benzyl-piperidin-4- yl)-amide |
| 190 | 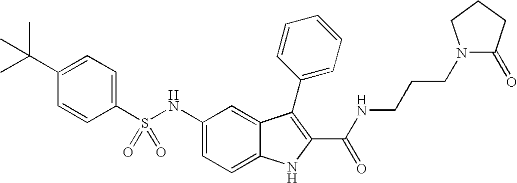 | 5-(4-tert-Butyl-benzenesulfonyl-amino)-3-phenyl- 1H-indole-2-carboxylic acid [3-(2-oxo-pyrrolidin-1- yl)-propyl]-amide |
| 191 | 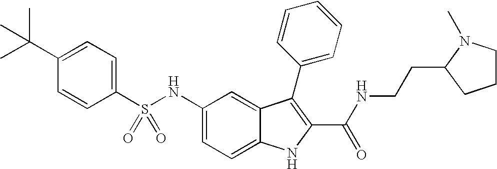 | 5-(4-tert-Butyl-benzenesulfonyl-amino)-3-phenyl- 1H-indole-2-carboxylic acid [2-(1-methyl- pyrrolidin-2-yl)-ethyl]-amide |
| 192 | 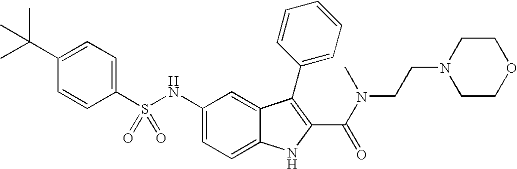 | 5-(4-tert-Butyl-benzenesulfonyl-amino)-3-phenyl- 1H-indole-2-carboxylic acid methyl-(2-morpholin-4- yl-ethyl)-amide |

### BIOLOGICAL EXAMPLES

### Example 1

### sAC-Assay

In a suitable buffer system, the soluble, sperm-specific adenylate cyclase catalyzes the reaction of adenosine triphosphate (ATP) to cyclic adenosine monophosphate (cAMP) and pyrophosphate. Free cAMP that is generated in this way is then used in a competitive detection process, in which the binding of a europium kryptate (Eu[K])-labeled anti-cAMP antibody (anti cAMP-Eu[K]-AK) to a cAMP-molecule-labeled, modified allophycocyanine-1 molecule (cAMP-XL665) is prevented. In the absence of exogenic cAMP, after excitation at 335 nm, it results in a fluorescence resonance energy transfer (FRET) between the anti cAMP-Eu[K]-AK (FRET-donor) and the cAMP-XL665 molecule (FRET-acceptor). This process is quantified at different times (time-resolved) based on the emission of FRET-acceptor XL665 (665 nm and 620 nm). A signal drop (measured as a wave ratio; calculation formula: [(E665 nm/E620 nm) X 10000]) can be attributed to the presence of cAMP and thus to the activity of sAC.

First, 1.5 μl of the test substance (in 30% DMSO), only 30% DMSO in the solvent controls, is introduced per recess in a 384-hole test plate (polystyrene; 384, NV). Then, 10 μl of a dilute sAC enzyme solution is recovered (enzyme stock solution in 300 mmol of NaCl, 10% glycerol; pH 7.6; intermediate and final enzyme dilution a) 1:10 and b) 1:2000 in each case in: 1.0 mmol of MnCl₂; 0.2% BSA; 50 mmol of tris, pH 7.5 in H₂O). The enzyme reaction is started by adding 5 μl of the ATP-substrate solution (200 μmol of ATP in H₂O) and is completed after incubation (25 minutes at room temperature) by the addition of 5 μl of stop solution (200 μmol of EDTA in PBS). Finally, the entire reaction is adjusted to a total volume of 91.5 μl by adding 70 μl of PBS.

Then, 8 μl of detection solution 1 is introduced into a recess of the 384-hole measuring plate (measuring plate: polystyrene; 384, SV-black; detection solution 1: 50 μl of cAMP-XL665; 950 PI of reconstitution buffer; 2200 PI of PBS; cAMP-XL665: production by the addition of 5 ml of H₂O to the freeze-dried product as specified by Cis Bio Kit: #62AMPPEC instructions; storage: aliquoted at −80° C.). Then, 3 PI from the 91.5 μl is added to the corresponding recess of the test plate. Finally, the addition of 8 μl of detection solution 2 (detection solution 2: 50 μl of anti cAMP-Eu[K]-AK; 950 μl of reconstitution buffer; 2200 μl of PBS; anti cAMP-Eu[K]-AK: production as specified by Cis Bio Kit: #62AMPPEC instructions; storage: aliquoted at −80° C.) is carried out.

After an additional incubation of 90 minutes at room temperature, the HTRF result is measured either on the Packard Discovery or with the RubiStar HTRF measuring device (Delay: 50 μs; Integration time: 400 μs).

### Example 2

### Isolation of Human Sperm from Ejaculates and Capacitation

2.1. Isolation of Sperm:

Human sperm are purified from the ejaculate by a two-layer gradient system based on colloidal silica particles (trade name: Percoll or ISolate).

Per ejaculate, 2.5 ml each of a preheated lower layer (“90% ISolate lower layer,” Irvine Company) is introduced into a 15 ml centrifuging tube (conical, plastic) and carefully covered with 2.5 ml of a preheated upper layer (“50% ISolate upper layer,” Irvine Company) and held back in a water bath at 37° C. for <1 hour. The gradient is carefully coated with a maximum of 3 ml of normal (relative to the number of sperm, motility and liquefaction) ejaculate. The sedimentation of sperm is carried out at 1000×g for 25 minutes at room temperature. By means of a glass capillary, both layers are suctioned off to a point just above the sperm pellets. To wash out the Isolate gradients, the sperm pellets that are resuspended in about 200 μl each are moved into a 15 ml plastic tube with 12 ml of mHTF medium (4 mmol of NaHCO₃; 0.01% BSA; 37° C.), and the sperm are sedimented at 1000×g for 20 minutes. The medium is suctioned off to a point just above the pellet and adjusted to 1000 μl with mHTF medium (4 mmol of NaHCO₃; 0.01% BSA; 37° C.). The number of sperm is determined in a Neubauer counting chamber, and adjusted for the following capacitation optionally with mHTF medium (4 mmol of NaHCO₃; 0.01% BSA; 37° C.) to 4×106 sperm/150 μl.

2.2. Capacitation

If the effect of test substances on the acrosomal reaction is to be tested, the sperms must be pre-incubated with the test substances. This pre-incubation (15 minutes in the incubator at 37° C.) is necessary to make possible the penetration of test substances in the sperm before the beginning of capacitation, i.e., to achieve a pre-saturation of the binding sites in the sperm, in particular in substances that do not pass through the membrane well. In addition, it is necessary since the increase of the BSA concentration in the capacitation by the high lipid bond of the BSA could result in the reduction of the effective test substance concentration in the preparation.

The test substances are dissolved in DMSO and diluted with mHTF medium (4 mmol of NaHCO₃; 0.01% BSA; 37° C.), such that in the final capacitation preparation of 400 μl, the DMSO concentration is 0.5%. 150 μl each of the tempered test substance solution above is pipetted in each case into 150 μl of sperm suspension and pre-incubated for 15 minutes at 37° C. The capacitation of sperm is started by adding 100 μl of mHTF-medium (88 mmol of NaHCO₃; 4% BSA; 37° C.). In the final 400 PI of capacitation preparation, the sperm concentration is 10×106/ml, the bicarbonate concentration is 4 mmol, and the BSA concentration is 1%. The capacitation is carried out at 37° C. for 3 hours in an incubator.

To complete the capacitation, each of the preparations (400 μl each) is transferred completely into a 15 ml sample tube with 1.5 ml of mHTF (4 mmol of NaHCO₃; 37° C.), centrifuged for 5 minutes at 1000×g, and the supernatant is removed. With this step, both the high amount of protein and the test substances are removed.

### Example 3

### Flow-Cytometric Determination of the Acrosomal Reaction

3.1. Introduction of the Acrosomal Reaction by Ionophore Treatment and Simultaneous CD46-FITC Staining

The acrosomal reaction (AR) of the sperm is triggered by the binding of the sperm to the Zona pellucida (ZP). In this case, enzymes are released from the acrosome that make it possible for the sperm to penetrate the ovocyte through the ZP. In the case of AR, in sperm, it results in a partial merging of the plasma membrane with the outside acrosomal membrane (OAM). The head of the sperm cell is limited only by the inside acrosomal membrane (IAM) at the end. The CD46-antigen can be detected only on the IAM.

The acrosomal reaction can be induced in vitro with a suitable concentration of the calcium-ionophore A23187 on capacitated sperm, but not on uncapacitated sperm or on sperm that are inhibited in capacitation by test substances. With the aid of FITC-labeled anti-CD46 antibodies (Pharmingen Company) against the IAM, the acrosome-reacted sperm can be distinguished in the flow cytometer from the acrosome-intact sperm, in which the IAM is not exposed. By the simultaneous staining of the sperm with the DNA dye ethidium homodimer (EhD), which stains only the DNA membrane-defective, thus dead cells, the dead sperm can be distinguished from the living sperm.

Since the ionophore dilutions seem to be very unstable in triggering the AR and must be mixed for the simultaneous staining with the CD46-FITC solution, the solutions cannot be prepared before the beginning of the test but rather must be produced during the working-up of the capacitation preparations.

The sperm pellets are resuspended in the residual supernatant and are diluted in a water bath (37° C.) with 450 μl of mHTF (4 mmol of NaHCO₃; 0.01% BSA; 37° C.). 100 μl Aliquots of the sperm suspensions are pipetted into prepared FACS-flow tube samples (in a water bath). 150 μl of a solution with ionophore and FITC-labeled anti-CD46 antibodies are pipetted into the sperm. The final concentration is 800 nmol of ionophore and a 1:125 dilution of the anti-CD46 antibody in mHTF (4 mmol of NaHCO₃; 0.01% BSA; 37° C.). The sperm are incubated therein, protected from light, for 30 minutes in a water bath at 37° C.

The incubation is stopped by adding 3.5 ml of PBS [0.1% BSA]/preparation, followed by centrifuging for 5 minutes at 700×g (room temperature) and subsequent suctioning-off of the supernatants. After the centrifuging, the samples are kept warm on the hot plate until measurement is done.

3.2. EhD Staining (for Differentiation of Dead/Living Acrosomally-Reacted Sperm)

After suctioning-off, the sperm pellets are mixed with 500 μl each of freshly prepared EhD solution (150 nmol of EhD in PBS [w/o BSA]; 37° C.). The samples can then be measured in a flow cytometer (BD Facs Calibur). The measurement is done at a laser excitation wavelength of 488 nm; 10,000 sperm per measurement are detected. Acrosome-reacted sperm are measured with CD46-FITC in an FL-1 filter at 530 nm. Dead sperm are measured by means of EhD-DNA-staining in an FL-2 filter at 634 nm. The measuring channels are first compensated appropriately with respect to one another.

3.3 Evaluation:

The sperm are selected as a very uniform cell population in an FSC—H (forward scatter) from SSC—H (sideward scatter) Dotblot. Since a two-color fluorescence staining is used, the evaluation is carried out with the aid of a quadrant analysis in an FL-1 (EhD, X-axis) vs. FL-2 (FITC-CD46, Y-axis) Dotblot with the selected sperm population from the FSC vs SSC Dotblot:

| | Quadrant in FL-1 | | |
|---|---|---|---|
| | vs. FL-2 Dotblot | Staining | Analysis |
| Q1 = UL | upper left | Only EhD | Dead, non-acrosomally-reacted |
| | | | sperm |
| Q2 = UR | upper right | EhD and | Dead, acrosomally-reacted sperm |
| | | FITC-CD46 | |
| Q3 = LL | lower left | Unstained | Living, non-acrosomally-reacted |
| | | | sperm |
| Q4 = LR | lower right | Only | Living, acrosomally-reacted sperm |
| | | FITC-CD46 | |

To calculate the % of induced, acrosomally-reacted sperm (=“IAR[%]”), only the living sperm from Q3 and Q4 are used, and their total number is set at equal to 100%. IAR is then calculated as follows:

$$IAR [ \% ] = \frac{LR \times 100}{LL + LR}$$

A portion of the sperm already reacts spontaneously acrosomally without the addition of ionophore (=“SAR[%]”). Therefore, a control measurement of identically-treated sperm without the addition of an ionophore is always also taken. The SAR is calculated analogously to the IAR. The acrosomal reaction (=“ARIC[%]”) that is actually triggered by the ionophore is calculated as the difference: ARIC=IAR−SAR.

For the following analysis of the effect of our inhibitors on the sAC-mediated capacitation (measured as the ability of the sperm to undergo ionophore-induced acrosomal reaction), the percentage of acrosomally-reacted sperm in the positive capacitation control (=incubation with mHTF medium with 25 mmol of NaHCO3; 1% BSA without test substances) is set at =100%. The ability of the sperm mixed with the test substances to undergo acrosomal reaction is indicated relative to this maximum acrosomal reaction.

Materials Used:

mHTF=modif. human tubular fluid (Irvine Scientific Company), Dulbecco's phosphate-buffered saline (Gibco Company) (with Ca²⁺, Mg^{2+, 1} g/l of D-glucose, 36 mg/l of Na-pyruvate, w/o phenol red, w/o NaHCO₃); bovine serum albumin, Fraction V (Fluka Company); dimethyl sulfoxide (DMSO), anhydrous (Merck Company); Sodium Bicarbonate 7.5% solution (893 mmol) (Irvine Scientific Company); isolate gradient (Irvine Scientific Company); Ionophore-A23187 free acid, (Calbiochem Company); ethidium homodimer (EhD) (Molecular Probe Company), Mouse Anti Human CD46:FITC (Pharmingen Company).

### BIBLIOGRAPHICAL REFERENCES

J. W. Carver-Ward, Human Reproduction Vol. 11, No. 9, pp:1923 ff, 1996 High Fertilization Prediction by Flow Cytometric Analysis of the CD46 Antigen on the Inner Acrosomal Membrane of Spermatozoa
O. J. D'Cruz, G. G. Haas, Fertility and Sterility Vol. 65, No. 4, pp: 843 ff, 1996 Fluorescence-Labeled Fucolectins are Superior Markers for Flow Cytometric Quantitation of the Sperm Acrosome Reaction
E. Nieschlag, H. M. Behre, Andrologie [Andrology], Springer Verlag 1996

Biological Data

| # | IC₅₀ [M] | Solubility (g/l) |
|---|---|---|
| 1 | 3.4E−6 | 0.001 |
| 2 | 4.9E−6 | 0.001 |
| 3 | 2.0E−6 | 0.001 |
| 6 | 3.7E−7 | 0.0001 |
| 9 | 5.3E−6 | 0.0015 |
| 10 | 2.2E−6 | 0.0021 |
| 19 | 1.5E−7 | 0.004 |
| 20 | 2.6E−6 | |
| 21 | 3.4E−6 | 0.0001 |
| 24 | 1.6E−6 | 0.008 |
| 25 | 6.3E−7 | 0.005 |
| 26 | 9.9E−6 | 0.003 |
| 28 | 2.7E−6 | 0.005 |
| 29 | 2E−6 | 0.007 |
| 31 | 1.2E−6 | 0.007 |
| 32 | 1.3E−6 | 0.055 |
| 37 | 7E−8 | 0.013 |
| 40 | 8.6E−8 | 0.005 |

Comparison with Known Compounds

The compounds according to the invention were compared to known compounds in the enzyme test. The result is indicated in the following table.

| | | | IC50 | Solubility |
|---|---|---|---|---|
| Example | R4 | R3 | [M] | (g/l) |
| 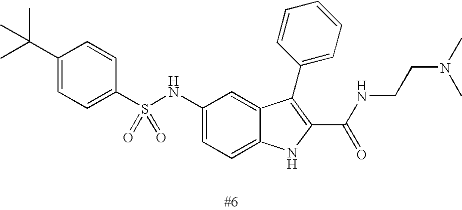 | (CH₂)₂N(CH₃)₂ | Phenyl | 3.7E − 7 | 0.0001 |
| 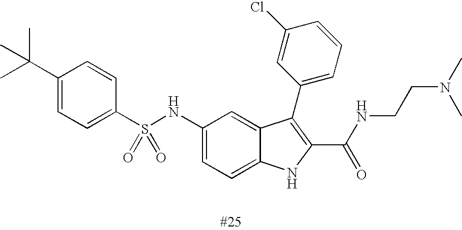 | CH₂)₂N(CH₃)₂ | Cl-Phenyl | 6.3E − 7 | 0.005 |
| 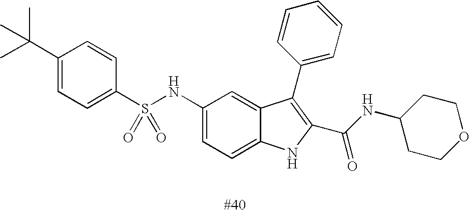 | 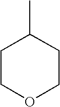 | Phenyl | 8.6E − 8 | 0.005 |
| 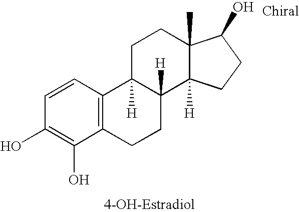 | | | 1.3E − 5 | |
| 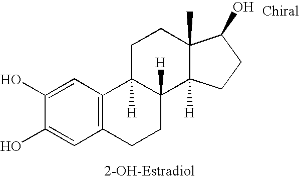 | | | 1.1E − 5 | |

It can be seen from the table that the compounds according to the invention relative to the inhibition of the soluble adenylate cyclase, expressed by the IC₅₀ value, sometimes have a 150× higher activity than the already known catechol estrogens (OH-estradiols). The catechol estrogens are toxic, therefore the compounds according to the invention are far superior to the known compounds. The compounds according to the invention are also about 100× more powerful than the compounds presented by Zippin.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding German application No. 10 2004 047 272.6, filed Sep. 24, 2005, and U.S. Provisional Application Ser. No. 60/614,527, filed Oct. 1, 2004, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A compound of formula I 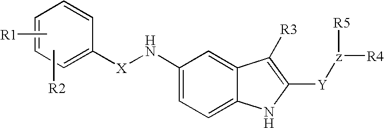
in which
R¹ stands for hydrogen, halogen, CF₃, C₃-C₆-cycloalkyl, which is optionally polysaturated and is optionally polysubstituted, or for the group C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl, C₁-C₆-aryl-C₁-C₆-alkyl or CF₃, in which C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl or C₁-C₆-aryl-C₁-C₆-alkyl, is optionally interrupted in one or more places, in the same way or differently, by oxygen, sulfur or nitrogen, or for the group sulfonyl-C₁-C₆-alkyl, sulfonamide, or cyano,
R² stands for halogen, CF₃, C₃-C₆-cycloalkyl, which is optionally polysaturated and is optionally polysubstituted, or for the group C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl, C₁-C₆-aryl-C₁-C₆-alkyl or CF₃, in which C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl or C₁-C₆-aryl-C₁-C₆-alkyl, is optionally interrupted in one or more places, in the same way or differently, by oxygen, sulfur or nitrogen, or for the group sulfonyl-C₁-C₆-alkyl, sulfonamide, or cyano,
R³ stands for C₆-C₁₂-aryl, which is optionally substituted in one or more places, in the same way or differently, with halogen, with C₁-C₆-alkyl or C₁-C₆-acyl, which is optionally substituted in one or more places, or can be substituted with C₁-C₆-alkoxy, hydroxy, cyano, CO₂—(C₁-C₆-alkyl), N—(C₁-C₆-alkyl)₂, CO—NR⁴R⁵ or with CF₃, for C₅-C₁₂-heteroaryl, which is optionally substituted in one or more places, in the same way or differently, with halogen, with C₁-C₆-alkyl, C₁-C₆-acyl, C₁-C₆-alkoxy, hydroxy, cyano, CO₂—(C₁-C₆-alkyl), N—(C₁-C₆-alkyl)₂, CO—NR⁴R⁵ or with CF₃, or for C₃-C₆-cycloalkyl, which is optionally substituted in one or more places, in the same way or differently, with halogen, CF₃, hydroxy, cyano, CO₂—(C₁-C₆-alkyl), C₁-C₆-alkyl, C₁-C₆-acyl, N—(C₁-C₆-alkyl)₂, CO—NR⁴R⁵ or C₁-C₆-alkoxy,
R⁴ stands for hydrogen, C₃-C₆-cycloalkyl, which is optionally substituted in one or more places, in the same way or differently, with C₁-C₆-alkyl, C₁-C₆-acyl, C₁-C₆-alkoxy or CF₃, for C₆-C₁₂-aryl, which is optionally substituted in one or more places, in the same way or differently, with halogen, with C₁-C₆-alkyl, C₁-C₆-acyl, C₁-C₆-alkoxy, N—C₁-C₆-alkyl-C₁-C₆-alkyl, CF₃ or cyano, or for C₅-C₁₂-heteroaryl, which is optionally substituted in one or more places, in the same way or differently, with halogen, with C₁-C₆-alkyl, C₁-C₆-acyl, C₁-C₆-alkoxy, N—C₁-C₆-alkyl-C₁-C₆-alkyl, CF₃ or cyano, or for C₁-C₆-alkyl, which can be substituted in any way desired,
R⁵ stands for hydrogen, C₃-C₆-cycloalkyl, which is optionally substituted in one or more places, in the same way or differently, with C₁-C₆-alkyl, C₁-C₆-acyl, C₁-C₆-alkoxy or CF₃, for C₆-C₁₂-aryl, which is optionally substituted in one or more places, in the same way or differently, with halogen, with C₁-C₆-alkyl, C₁-C₆-acyl, C₁-C₆-alkoxy, N—C₁-C₆-alkyl-C₁-C₆-alkyl, CF₃ or cyano, or for C₅-C₁₂-heteroaryl, which is optionally substituted in one or more places, in the same way or differently, with halogen, with C₁-C₆-alkyl, C₁-C₆-acyl, C₁-C₆-alkoxy, N—C₁-C₆-alkyl-C₁-C₆-alkyl, CF₃ or cyano, or for C₁-C₆-alkyl, which can be substituted in any way desired, or
R⁴ and R⁵ together form a 5- to 8-membered ring, which can contain additional heteroatoms, X stands for sulfonyl, (CH₂)ₙ or carbonyl,
Y stands for carbonyl, or (CH₂)ₙ,
Z stands for nitrogen, and
n stands for 0-4.

2. A compound according to claim 1, wherein
R¹ stands for hydrogen, halogen, CF₃, C₃-C₆-cycloalkyl, which is optionally polysaturated and is optionally polysubstituted, or for the group C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl, C₁-C₆-aryl-C₁-C₆-alkyl or CF₃, in which C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl or C₁-C₆-aryl-C₁-C₆-alkyl, is optionally interrupted in one or more places, in the same way or differently, by oxygen, sulfur or nitrogen, or for the group sulfonyl-C₁-C₆-alkyl, sulfonamide, or cyano,
R² stands for halogen, CF₃, or C₃-C₆-cycloalkyl, which is optionally polysaturated and is optionally polysubstituted, or for the group C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl, C₁-C₆-aryl-C₁-C₆-alkyl or CF₃, in which C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl or C₁-C₆-aryl-C₁-C₆-alkyl, is optionally interrupted in one or more places, in the same way or differently, by oxygen, sulfur or nitrogen, or for the group sulfonyl-C₁-C₆-alkyl, sulfonamide, or cyano,
R³ stands for C₆-C₁₂-aryl, which is optionally substituted in one or more places, in the same way or differently, with halogen, with C₁-C₆-alkyl, C₁-C₃-acyl, C₁-C₃-alkoxy, cyano, hydroxy, N—(CH₃)₂, CO₂—(C₁-C₃-alkyl), CO—NR⁴R⁵ or with CF₃, C₅-C₁₂-heteroaryl, which is optionally substituted in one or more places, in the same way or differently, with chlorine and/or fluorine, with C₁-C₆-alkyl, C₁-C₃-acyl, C₁-C₃-alkoxy, cyano, hydroxy, N—(CH₃)₂, CO₂—(C₁-C₃-alkyl), CO—NR⁴R⁵ or with CF₃, C₃-C₆-cycloalkyl, which is optionally substituted in one or more places, in the same way or differently, with chlorine and/or fluorine, CF₃, cyano, C₁-C₃-alkyl, C₁-C₃-acyl, hydroxy, N—(CH₃)₂, CO₂—(C₁-C₃-alkyl), CO—NR⁴R⁵ or C₁-C₃-alkoxy,
R⁴ stands for hydrogen, C₃-C₆-cycloalkyl, which is optionally substituted in one or more places, in the same way or differently, with C₁-C₃-alkyl, C₁-C₃-acyl, C₁-C₃-alkoxy or CF₃, for C₆-C₁₂-aryl, which is optionally substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₅-C₁₂-heteroaryl, which is optionally substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₁-C₆-alkyl, which can be substituted in any way desired,
R⁵ stands for hydrogen, C₃-C₆-cycloalkyl, which is optionally substituted in one or more places, in the same way or differently, with C₁-C₃-alkyl, C₁-C₃-acyl, C₁-C₃-alkoxy or CF₃, for C₆-C₁₂-aryl, which is optionally substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₅-C₁₂-heteroaryl, which is optionally substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₁-C₆-alkyl, which can be substituted in any way desired, or
R⁴ and R⁵ together form a 5- to 8-membered ring, which can contain additional heteroatoms,
X stands for sulfonyl, (CH₂)ₙ or carbonyl,
Y stands for carbonyl, or (CH₂)ₙ,
Z stands for nitrogen, and
n stands for 0-2.

3. A compound according to claim 1, wherein
R¹ stands for hydrogen,
R² stands for tert-butyl, cyano, bromine, or for the group —O—CF₃, —SO₂—CH₃ and is in para-position,
R³ stands for one of the following groups 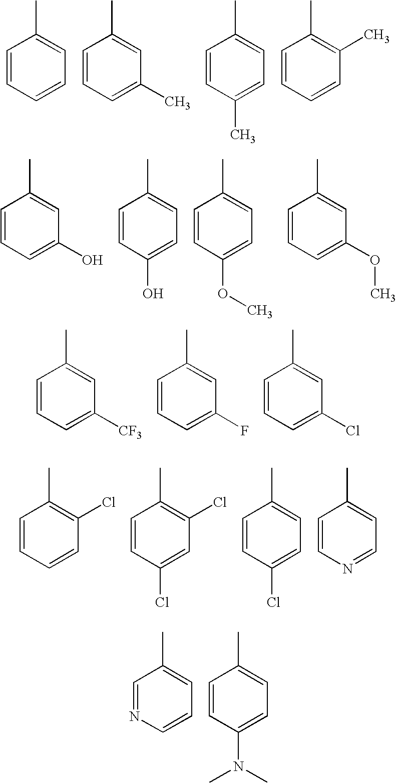
R⁴ stands for hydrogen or for one of the following groups —(CH₂)ₙ—N—(CH₃)₂, —(CH₂)₂—CH₃, —(CH₂)₂—NH—COCH₃, —(CH₂)—CHCH₃—OH, —(CH₂)₂—O—CH₃, —(CH₂)₂—OH, —CHCH₃—CH₂—OH, 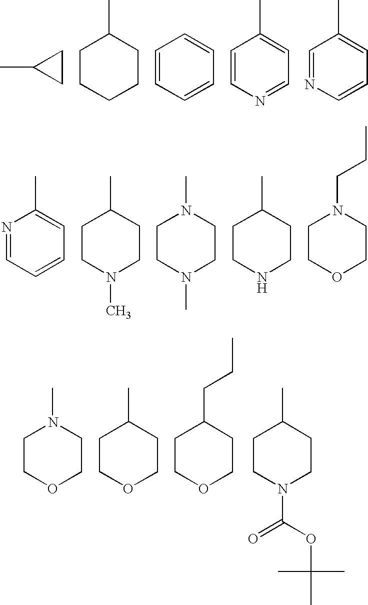
R⁵ stands for hydrogen,
X stands for sulfonyl, carbonyl or CH₂,
Y stands for carbonyl, or for the group (CH₂)ₙ,
Z stands for nitrogen or for one of the following groups 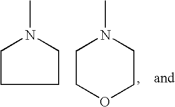
n is 1-2.

4. A compound according to claim 1, wherein
R¹ stands for hydrogen, tert-butyl, cyano, bromine, or for the group —O—CF₃, or —SO₂—CH₃,
R² stands for tert-butyl, cyano, bromine, or for the group —O—CF₃ or —SO₂—CH₃, and
R³ stands for C₆-C₁₂-aryl, which is optionally substituted in one or more places, in the same way or differently, with halogen, with C₁-C₆-alkyl, C₁-C₃-acyl, C₁-C₃-alkoxy, cyano, hydroxy, N—(CH₃)₂, CO₂—(C₁-C₃-alkyl), CO—NR⁴R⁵ or with CF₃, C₅-C₁₂-heteroaryl, which is optionally substituted in one or more places, in the same way or differently, with chlorine and/or fluorine, with C₁-C₆-alkyl, C₁-C₃-acyl, C₁-C₃-alkoxy, cyano, hydroxy, N—(CH₃)₂, CO₂—(C₁-C₃-alkyl), CO—NR⁴R⁵ or with CF₃, C₃-C₆-cycloalkyl, which is optionally substituted in one or more places, in the same way or differently, with chlorine and/or fluorine, CF₃, cyano, C₁-C₃-alkyl, C₁-C₃-acyl, hydroxy, N—(CH₃)₂, CO₂—(C₁-C₃-alkyl), CO—NR⁴R⁵ or C₁-C₃-alkoxy,
R⁴ stands for hydrogen, C₃-C₆-cycloalkyl, which is optionally substituted in one or more places, in the same way or differently, with C₁-C₃-alkyl, C₁-C₃-acyl, C₁-C₃-alkoxy or CF₃, for C₆-C₁₂-aryl, which is optionally substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₅-C₁₂-heteroaryl, which is optionally substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₁-C₆-alkyl, which can be substituted in any way desired,
R⁵ stands for hydrogen, C₃-C₆-cycloalkyl, which is optionally substituted in one or more places, in the same way or differently, with C₁-C₃-alkyl, C₁-C₃-acyl, C₁-C₃-alkoxy or CF₃, for C₆-C₁₂-aryl, which is optionally substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₅-C₁₂-heteroaryl, which is optionally substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₁-C₆-alkyl, which can be substituted in any way desired, or
R⁴ and R⁵ together form a 5- to 8-membered ring, which can contain additional heteroatoms,
X stands for sulfonyl, (CH₂)ₙ or carbonyl,
Y stands for carbonyl, or (CH₂)ₙ,
Z stands for nitrogen, and
n stands for 0-2.

5. A compound according to claim 1, wherein
R¹ stands for hydrogen,
R² stands for tert-butyl, cyano, bromine, or for the group —O—CF₃ or —SO₂—CH₃ and is in para-position, and
R³ stands for C₆-C₁₂-aryl, which is optionally substituted in one or more places, in the same way or differently, with halogen, with C₁-C₆-alkyl, C₁-C₃-acyl, C₁-C₃-alkoxy, cyano, hydroxy, N—(CH₃)₂, CO₂—(C₁-C₃-alkyl), CO—NR⁴R⁵ or with CF₃, C₅-C₁₂-heteroaryl, which is optionally substituted in one or more places, in the same way or differently, with chlorine and/or fluorine, with C₁-C₆-alkyl, C₁-C₃-acyl, C₁-C₃-alkoxy, cyano, hydroxy, N—(CH₃)₂, CO₂—(C₁-C₃-alkyl), CO—NR⁴R⁵ or with CF₃, C₃-C₆-cycloalkyl, which is optionally substituted in one or more places, in the same way or differently, with chlorine and/or fluorine, CF₃, cyano, C₁-C₃-alkyl, C₁-C₃-acyl, hydroxy, N—(CH₃)₂, CO₂—(C₁-C₃-alkyl), CO—NR⁴R⁵ or C₁-C₃-alkoxy,
R⁴ stands for hydrogen, C₃-C₆-cycloalkyl, which is optionally substituted in one or more places, in the same way or differently, with C₁-C₃-alkyl, C₁-C₃-acyl, C₁-C₃-alkoxy or CF₃, for C₆-C₁₂-aryl, which is optionally substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₅-C₁₂-heteroaryl, which is optionally substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₁-C₆-alkyl, which can be substituted in any way desired,
R⁵ stands for hydrogen, C₃-C₆-cycloalkyl, which is optionally substituted in one or more places, in the same way or differently, with C₁-C₃-alkyl, C₁-C₃-acyl, C₁-C₃-alkoxy or CF₃, for C₆-C₁₂-aryl, which is optionally substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₅-C₁₂-heteroaryl, which is optionally substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₁-C₆-alkyl, which can be substituted in any way desired, or
R⁴ and R⁵ together form a 5- to 8-membered ring, which can contain additional heteroatoms,
X stands for sulfonyl, (CH₂)ₙ or carbonyl,
Y stands for carbonyl, or (CH₂)ₙ,
Z stands for nitrogen, and
n stands for 0-2.

6. A compound according to claim 1, wherein
R¹ stands for hydrogen, halogen, CF₃, C₃-C₆-cycloalkyl, which is optionally polysaturated and is optionally polysubstituted, or for the group C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl, C₁-C₆-aryl-C₁-C₆-alkyl or CF₃, in which C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl or C₁-C₆-aryl-C₁-C₆-alkyl, is optionally interrupted in one or more places, in the same way or differently, by oxygen, sulfur or nitrogen, or for the group sulfonyl-C₁-C₆-alkyl, sulfonamide, or cyano,
R² stands for halogen, CF₃, C₃-C₆-cycloalkyl, which is optionally polysaturated and is optionally polysubstituted, or for the group C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl, C₁-C₆-aryl-C₁-C₆-alkyl or CF₃, in which C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl or C₁-C₆-aryl-C₁-C₆-alkyl, is optionally interrupted in one or more places, in the same way or differently, by oxygen, sulfur or nitrogen, or for the group sulfonyl-C₁-C₆-alkyl, sulfonamide, or cyano,
R³ stands for C₆-C₁₂-aryl, which is optionally substituted in one or more places, in the same way or differently, with halogen, with C₁-C₆-alkyl, C₁-C₃-acyl, C₁-C₃-alkoxy, cyano, hydroxy, N—(CH₃)₂, CO₂—(C₁-C₃-alkyl), CO—NR⁴R⁵ or with CF₃, C₅-C₁₂-heteroaryl, which is optionally substituted in one or more places, in the same way or differently, with chlorine and/or fluorine, with C₁-C₆-alkyl, C₁-C₃-acyl, C₁-C₃-alkoxy, cyano, hydroxy, N—(CH₃)₂, CO₂—(C₁-C₃-alkyl), CO—NR⁴R⁵ or with CF₃, C₃-C₆-cycloalkyl, which is optionally substituted in one or more places, in the same way or differently, with chlorine and/or fluorine, CF₃, cyano, C₁-C₃-alkyl, C₁-C₃-acyl, hydroxy, N—(CH₃)₂, CO₂—(C₁-C₃-alkyl), CO—NR⁴R⁵ or C₁-C₃-alkoxy,
R⁴ stands for hydrogen, C₃-C₆-cycloalkyl, which is optionally substituted in one or more places, in the same way or differently, with C₁-C₆-alkyl, C₁-C₆-acyl, C₁-C₆-alkoxy or CF₃, for C₆-C₁₂-aryl, which is optionally substituted in one or more places, in the same way or differently, with halogen, with C₁-C₆-alkyl, C₁-C₆-acyl, C₁-C₆-alkoxy, N—C₁-C₆-alkyl-C₁-C₆-alkyl, CF₃ or cyano, or for C₅-C₁₂-heteroaryl, which is optionally substituted in one or more places, in the same way or differently, with halogen, with C₁-C₆-alkyl, C₁-C₆-acyl, C₁-C₆-alkoxy, N—C₁-C₆-alkyl-C₁-C₆-alkyl, CF₃ or cyano, or for C₁-C₆-alkyl, which can be substituted in any way desired,
R⁵ stands for hydrogen, C₃-C₆-cycloalkyl, which is optionally substituted in one or more places, in the same way or differently, with C₁-C₆-alkyl, C₁-C₆-acyl, C₁-C₆-alkoxy or CF₃, for C₆-C₁₂-aryl, which is optionally substituted in one or more places, in the same way or differently, with halogen, with C₁-C₆-alkyl, C₁-C₆-acyl, C₁-C₆-alkoxy, N—C₁-C₆-alkyl-C₁-C₆-alkyl, CF₃ or cyano, or for C₅-C₁₂-heteroaryl, which is optionally substituted in one or more places, in the same way or differently, with halogen, with C₁-C₆-alkyl, C₁-C₆-acyl, C₁-C₆-alkoxy, N—C₁-C₆-alkyl-C₁-C₆-alkyl, CF₃ or cyano, or for C₁-C₆-alkyl, which can be substituted in any way desired, or
R⁴ and R⁵ together form a 5- to 8-membered ring, which can contain additional heteroatoms,
X stands for sulfonyl, (CH₂)ₙ or carbonyl,
Y stands for carbonyl, or (CH₂)ₙ,
Z stands for nitrogen, and
n stands for 0-4.

7. A compound according to claim 1, wherein
R¹ stands for hydrogen,
R² stands for tert-butyl, cyano, bromine, or for the group —O—CF₃ or —SO₂—CH₃ and is in para-position,
R³ stands for C₆-C₁₂-aryl, which is optionally substituted in one or more places, in the same way or differently, with halogen, with C₁-C₆-alkyl, C₁-C₃-acyl, C₁-C₃-alkoxy, cyano, hydroxy, N—(CH₃)₂, CO₂—(C₁-C₃-alkyl), CO—NR⁴R⁵ or with CF₃, C₅-C₁₂-heteroaryl, which is optionally substituted in one or more places, in the same way or differently, with chlorine and/or fluorine, with C₁-C₆-alkyl, C₁-C₃-acyl, C₁-C₃-alkoxy, cyano, hydroxy, N—(CH₃)₂, CO₂—(C₁-C₃-alkyl), CO—NR⁴R⁵ or with CF₃, C₃-C₆-cycloalkyl, which is optionally substituted in one or more places, in the same way or differently, with chlorine and/or fluorine, CF₃, cyano, C₁-C₃-alkyl, C₁-C₃-acyl, hydroxy, N—(CH₃)₂, CO₂—(C₁-C₃-alkyl), CO—NR⁴R⁵ or C₁-C₃-alkoxy, R⁴ stands for hydrogen or for one of the following groups —(CH₂), —N—(CH₃)₂, —(CH₂)₂—CH₃, —(CH₂)₂—NH—COCH₃, —(CH₂)—CHCH₃—OH, —(CH₂)₂—O—CH₃, —(CH₂)₂—OH, —CHCH₃—CH₂—OH, 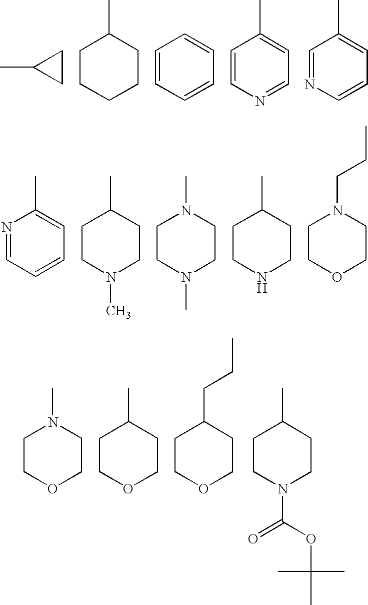
R⁵ stands for hydrogen,
X stands for sulfonyl, carbonyl or CH₂,
Y stands for carbonyl, or for the group (CH₂)ₙ,
Z stands for nitrogen or for one of the following groups 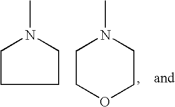
n stands for 1-2.

8. A compound according to claim 1, wherein
R¹ stands for hydrogen, halogen, CF₃, C₃-C₆-cycloalkyl, which is optionally polysaturated and is optionally polysubstituted, or for the group C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl, C₁-C₆-aryl-C₁-C₆-alkyl or CF₃, in which C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl or C₁-C₆-aryl-C₁-C₆-alkyl, is optionally can be interrupted in one or more places, in the same way or differently, by oxygen, sulfur or nitrogen, or for the group sulfonyl-C₁-C₆-alkyl, sulfonamide, or cyano,
R² stands for halogen, CF₃, C₃-C₆-cycloalkyl, which is optionally polysaturated and is optionally polysubstituted, or for the group C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl, C₁-C₆-aryl-C₁-C₆-alkyl or CF₃, in which C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl or C₁-C₆-aryl-C₁-C₆-alkyl, is optionally interrupted in one or more places, in the same way or differently, by oxygen, sulfur or nitrogen, or for the group sulfonyl-C₁-C₆-alkyl, sulfonamide, or cyano,
R³ stands for one of the following groups 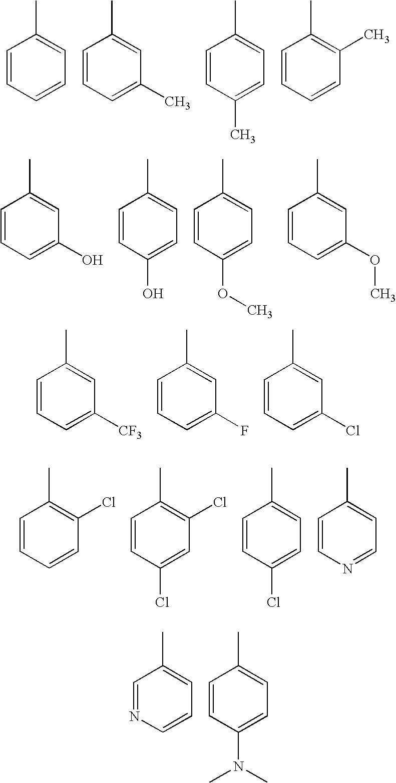
R⁴ stands for hydrogen, C₃-C₆-cycloalkyl, which is optionally substituted in one or more places, in the same way or differently, with C₁-C₃-alkyl, C₁-C₃-acyl, C₁-C₃-alkoxy or CF₃, for C₆-C₁₂-aryl, which is optionally substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₅-C₁₂-heteroaryl, which is optionally substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₁-C₆-alkyl, which can be substituted in any way desired,
R⁵ stands for hydrogen, C₃-C₆-cycloalkyl, which is optionally substituted in one or more places, in the same way or differently, with C₁-C₃-alkyl, C₁-C₃-acyl, C₁-C₃-alkoxy or CF₃, for C₆-C₁₂-aryl, which is optionally substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₅-C₁₂-heteroaryl, which is optionally substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₁-C₆-alkyl, which can be substituted in any way desired, or
R⁴ and R⁵ together form a 5- to 8-membered ring, which can contain additional heteroatoms,
X stands for sulfonyl, (CH₂)ₙ or carbonyl,
Y stands for carbonyl, or (CH₂)ₙ,
Z stands for nitrogen, and
n stands for 0-2.

9. A compound according to claim 1, wherein
R¹ stands for hydrogen,
R² stands for tert-butyl, cyano, bromine or for the group —O—CF₃ or —SO₂—CH₃ and is in para-position, and
R³ stands for one of the following groups 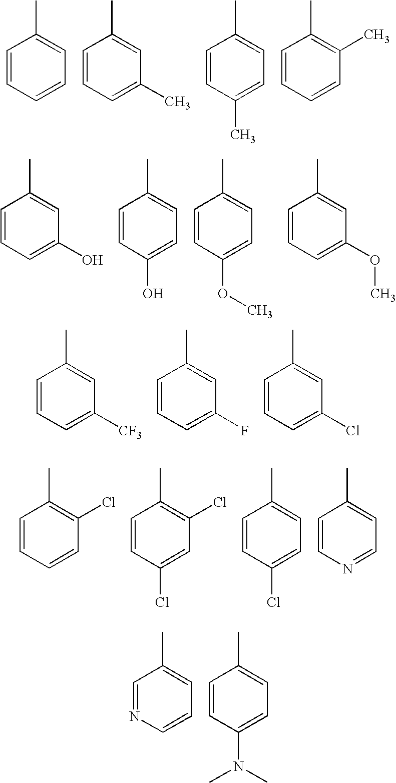
R⁴ stands for hydrogen, C₃-C₆-cycloalkyl, which is optionally substituted in one or more places, in the same way or differently, with C₁-C₃-alkyl, C₁-C₃-acyl, C₁-C₃-alkoxy or CF₃, for C₆-C₁₂-aryl, which is optionally substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₅-C₁₂-heteroaryl, which is optionally substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₁-C₆-alkyl, which can be substituted in any way desired,
R⁵ stands for hydrogen, C₃-C₆-cycloalkyl, which is optionally substituted in one or more places, in the same way or differently, with C₁-C₃-alkyl, C₁-C₃-acyl, C₁-C₃-alkoxy or CF₃, for C₆-C₁₂-aryl, which is optionally substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₅-C₁₂-heteroaryl, which is optionally substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₁-C₆-alkyl, which can be substituted in any way desired,
X stands for sulfonyl, carbonyl or CH₂,
Y stands for carbonyl, or for the group (CH₂)ₙ,
Z stands for nitrogen or for one of the following groups 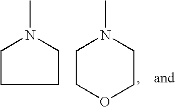
n stands for 1-2.

10. A compound according to claim 1, wherein
R¹ stands for hydrogen, halogen, CF₃, C₃-C₆-cycloalkyl, which is optionally polysaturated and is optionally polysubstituted, or for the group C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl, C₁-C₆-aryl-C₁-C₆-alkyl or CF₃, in which C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl or C₁-C₆-aryl-C₁-C₆-alkyl, is optionally interrupted in one or more places, in the same way or differently, by oxygen, sulfur or nitrogen, or for the group sulfonyl-C₁-C₆-alkyl, sulfonamide, or cyano,
R² is halogen, CF₃, C₃-C₆-cycloalkyl, which is optionally polysaturated and is optionally polysubstituted, or for the group C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl, C₁-C₆-aryl-C₁-C₆-alkyl or CF₃, in which C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl or C₁-C₆-aryl-C₁-C₆-alkyl, is optionally interrupted in one or more places, in the same way or differently, by oxygen, sulfur or nitrogen, or for the group sulfonyl-C₁-C₆-alkyl, sulfonamide, or cyano,
R³ stands for C₆-C₁₂-aryl, which is optionally substituted in one or more places, in the same way or differently, with halogen, with C₁-C₆-alkyl, C₁-C₃-acyl, C₁-C₃-alkoxy, cyano, hydroxy, N—(CH₃)₂, CO₂—(C₁-C₃-alkyl), CO—NR⁴R⁵ or with CF₃, C₅-C₁₂-heteroaryl, which is optionally substituted in one or more places, in the same way or differently, with chlorine and/or fluorine, with C₁-C₆-alkyl, C₁-C₃-acyl, C₁-C₃-alkoxy, cyano, hydroxy, N—(CH₃)₂, CO₂—(C₁-C₃-alkyl), CO—NR⁴R⁵ or with CF₃, C₃-C₆-cycloalkyl, which is optionally substituted in one or more places, in the same way or differently, with chlorine and/or fluorine, CF₃, cyano, C₁-C₃-alkyl, C₁-C₃-acyl, hydroxy, N—(CH₃)₂, CO₂—(C₁-C₃-alkyl), CO—NR⁴R⁵ or C₁-C₃-alkoxy,
R⁴ stands for hydrogen or for one of the following groups —(CH₂), —N—(CH₃)₂, —(CH₂)₂—CH₃, —(CH₂)₂—NH—COCH₃, —(CH₂)—CHCH₃—OH, —(CH₂)₂—O—CH₃, —(CH₂)₂—OH, —CHCH₃—CH₂—OH, 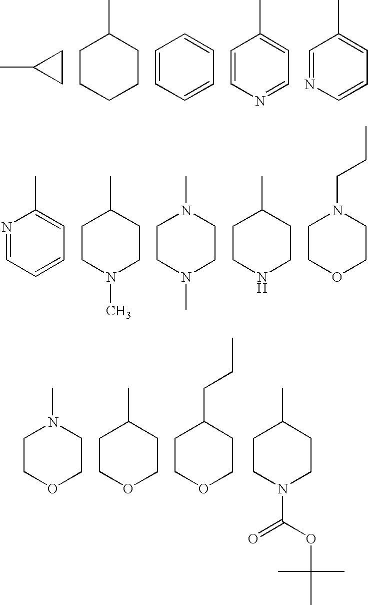
R⁵ stands for hydrogen,
X stands for sulfonyl, (CH₂)ₙ or carbonyl,
Y stands for carbonyl, or (CH₂)ₙ,
Z stands for nitrogen, and
n stands for 0-2.

11. A compound according to claim 1, wherein
R¹ stands for hydrogen, halogen, CF₃, C₃-C₆-cycloalkyl, which is optionally polysaturated and is optionally polysubstituted, or for the group C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl, C₁-C₆-aryl-C₁-C₆-alkyl or CF₃, in which C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl or C₁-C₆-aryl-C₁-C₆-alkyl, is optionally interrupted in one or more places, in the same way or differently, by oxygen, sulfur or nitrogen, or for the group sulfonyl-C₁-C₆-alkyl, sulfonamide, or cyano,
R² is halogen, CF₃, C₃-C₆-cycloalkyl, which is optionally polysaturated and is optionally polysubstituted, or for the group C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl, C₁-C₆-aryl-C₁-C₆-alkyl or CF₃, in which C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl or C₁-C₆-aryl-C₁-C₆-alkyl, is optionally interrupted in one or more places, in the same way or differently, by oxygen, sulfur or nitrogen, or for the group sulfonyl-C₁-C₆-alkyl, sulfonamide, or cyano,
R³ stands for C₆-C₁₂-aryl, which is optionally substituted in one or more places, in the same way or differently, with halogen, with C₁-C₆-alkyl, C₁-C₃-acyl, C₁-C₃-alkoxy, cyano, hydroxy, N—(CH₃)₂, CO₂—(C₁-C₃-alkyl), CO—NR⁴R⁵ or with CF₃, C₅-C₁₂-heteroaryl, which is optionally substituted in one or more places, in the same way or differently, with chlorine and/or fluorine, with C₁-C₆-alkyl, C₁-C₃-acyl, C₁-C₃-alkoxy, cyano, hydroxy, N—(CH₃)₂, CO₂—(C₁-C₃-alkyl), CO—NR⁴R⁵ or with CF₃, C₃-C₆-cycloalkyl, which is optionally substituted in one or more places, in the same way or differently, with chlorine and/or fluorine, CF₃, cyano, C₁-C₃-alkyl, C₁-C₃-acyl, hydroxy, N—(CH₃)₂, CO₂—(C₁-C₃-alkyl), CO—NR⁴R⁵ or C₁-C₃-alkoxy,
R⁴ stands for hydrogen, C₃-C₆-cycloalkyl, which is optionally substituted in one or more places, in the same way or differently, with C₁-C₃-alkyl, C₁-C₃-acyl, C₁-C₃-alkoxy or CF₃, for C₆-C₁₂-aryl, which is optionally substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₅-C₁₂-heteroaryl, which is optionally substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₁-C₆-alkyl, which can be substituted in any way desired,
R⁵ stands for hydrogen, C₃-C₆-cycloalkyl, which is optionally substituted in one or more places, in the same way or differently, with C₁-C₃-alkyl, C₁-C₃-acyl, C₁-C₃-alkoxy or CF₃, for C₆-C₁₂-aryl, which is optionally substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₅-C₁₂-heteroaryl, which is optionally substituted in one or more places, in the same way or differently, with halogen, with C₁-C₃-alkyl, C₁-C₃-acyl, C₁-C₃-alkoxy, N—C₁-C₃-alkyl-C₁-C₃-alkyl, CF₃ or cyano, or for C₁-C₆-alkyl, which can be substituted in any way desired, or
R⁴ and R⁵ together form a 5- to 8-membered ring that can contain additional heteroatoms,
X stands for sulfonyl, (CH₂)ₙ or carbonyl,
Y stands for carbonyl, or (CH₂)ₙ,
Z stands for nitrogen or for one of the following groups 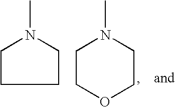
n stands for 0-2.

12. (canceled)

13. A pharmaceutical composition comprising a compound according to claim 1 and a pharmaceutically acceptable carrier.

14. A pharmaceutical composition according to claim 13 in which the compound of formula I is contained in an effective dose.

15. A method for treating a disease caused by a disorder in cAMP metabolism comprising administering to a subject in need thereof an effective amount of a compound of claim 1.

16. A method for non-hormonal contraception comprising administering to a subject in need thereof an effective amount of a compound of claim 1.

17. A method for inhibition of soluble adenylate cyclase comprising administering to a subject in need thereof an effective amount of a compound of claim 1.

18. (canceled)

19. A pharmaceutical composition comprising a compound of claim 1 and a pharmaceutically acceptable carrier, which composition is suitable for enteral, parenteral, vaginal or oral administration.

20. A compound according to claim 1, wherein
R⁴ stands for hydrogen, C₃-C₆-cycloalkyl, which is optionally substituted in one or more places, in the same way or differently, with C₁-C₆-alkyl, C₁-C₆-acyl, C₁-C₆-alkoxy or CF₃, for C₆-C₁₂-aryl, which is optionally substituted in one or more places, in the same way or differently, with halogen, with C₁-C₆-alkyl, C₁-C₆-acyl, C₁-C₆-alkoxy, N—C₁-C₆-alkyl-C₁-C₆-alkyl, CF₃ or cyano, or for C₅-C₁₂-heteroaryl, which is optionally substituted in one or more places, in the same way or differently, with halogen, with C₁-C₆-alkyl, C₁-C₆-acyl, C₁-C₆-alkoxy, N—C₁-C₆-alkyl-C₁-C₆-alkyl, CF₃ or cyano, or for C₁-C₆-alkyl, and
R⁵ stands for hydrogen, C₃-C₆-cycloalkyl, which is optionally substituted in one or more places, in the same way or differently, with C₁-C₆-alkyl, C₁-C₆-acyl, C₁-C₆-alkoxy or CF₃, for C₆-C₁₂-aryl, which is optionally substituted in one or more places, in the same way or differently, with halogen, with C₁-C₆-alkyl, C₁-C₆-acyl, C₁-C₆-alkoxy, N—C₁-C₆-alkyl-C₁-C₆-alkyl, CF₃ or cyano, or for C₅-C₁₂-heteroaryl, which is optionally substituted in one or more places, in the same way or differently, with halogen, with C₁-C₆-alkyl, C₁-C₆-acyl, C₁-C₆-alkoxy, N—C₁-C₆-alkyl-C₁-C₆-alkyl, CF₃ or cyano, or for C₁-C₆-alkyl.

21. A compound according to claim 1, which is 4-tert-Butyl-N-[3-phenyl-2-(pyrrolidine-1-carbonyl)-1H-indol-5-yl]-benzenesulfonamide; 4-tert-Butyl-N-[2-(morpholine-4-carbonyl)-3-phenyl-1H-indol-5yl]-benzene-sulfonamide; 5-(4-tert-Butylbenzylamino)-3-phenyl-1H-indole-2-carboxylic acid pyridin-4-ylamide; or 5-(4-tert-Butylbenzoylamino)-3-phenyl-1H-indole-2-carboxylic acid-(2-dimethylaminoethyl)amide.

22. A compound according to claim 1, wherein R⁴ and R⁵ together form a 5- to 8-membered ring, which can contain additional heteroatoms, and/or
X stands for (CH₂)ₙ or carbonyl.
